SEQUENCE LISTING (83 sequences)

SEQ ID NO: 1 - protein, 338 aa, Borrelia burgdorferi
MKSQVADKDDPTNKFYQSVIQLGNGFLDVFTSFGGLVAEAFGFKSDPKKSDVKTYFTTVAAKLEKTKTDLNSLPKEKSDISSTTGKPDSTGSVGTAVEGAIKEVSELLDKLVKAVKTAEGASSGTAAIGEVVADADAAKVADKASVKGIAKGIKEIVEAAGGSEKLKAVAAAKGENNKGAGKLFGKAGAAAHGDSEAASKAAGAVSAVSGEQILSAIVTAADAAEQDGKKPEEAKNPIAAAIGDKDGGAEFGQDEMKKDDQIAAAIALRGMAKDGKFAVKDGEKEKAEGAIKGAAESAVRKVLGAITGLIGDAVSSGLRKVGDSVKAASKETPPALNK

SEQ ID NO: 2 - protein, 357 aa, Borrelia burgdorferi
MNTKKISSAILLTTFFVFINCKSQVADKDDPTNKFYQSVIQLGNGFLDVFTSFGGLVAEAFGFKSDPKKSDVKTYFTTVAAKLEKTKTDLNSLPKEKSDISSTTGKPDSTGSVGTAVEGAIKEVSELLDKLVKAVKTAEGASSGTAAIGEVVDNAAAAKAADKDSVTGIAKGIKEIVEAAGGSKKLKAAAAKGENNKGAGKLFGKAGDAAHGDSEAASKAAGAVSAVSGEQILSAIVKAAAAGDQEGKKPGEAKNPIAAAIGEGDGDAEFNQDGMKKDDQIAAAIALRGMAKDGKFAVKNDEKGKAEGAIKGAAESAVRKVLGAITGLIGDAVSSGLRKVGDSVKAASKETPPALNK

SEQ ID NO: 3 - protein, 210 aa, Borrelia burgdorferi
MKKNTLSAILMTLFLFISCNNSGKDGNTSANSADESVKGPNLTEISKKITDSNAVLLAVKEVEALLSSIDEIAAKAIGKKIHQNNGLDTENNHNGSLLAGAYAISTLIKQKLDGLKNEGLKEKIDAAKKCSETFTNKLKEKHTDLGKEGVTDADAKEAILKTNGTKTKGAEELGKLFESVEVLSKAAKEMLANSVKELTSPVVAESPKKP

SEQ ID NO: 4 - protein, 250 aa, Borrelia burgdorferi
MSKLILAISILLIISCKWHVDNPIDEATAESKSALTSVDQVLDEISEATGLSSEKITKLTPEELENLAKEAQDDSEKSKKEIEDQKNTKESKNIEVKDTPRLIKLIKNSSEKIDSVFQTLINIGYNATYAAKSNLKNGLKMVKLLDELLKISVSSNGDKSTQKYNELKTVVNRFNAENSAIKVPLENGSKIEAKKCIKTLMTNVETYFKGVSTELKDKKDDKYTKILAALSEAANKIENAAMAIHLCFNN

SEQ ID NO: 5 - protein, 107 aa, Borrelia burgdorferi
RAGWIGDYADPLTFLSIFTQGYTQFSSHNYSNPEYNELIKKSDLELDPIKRQDILRQAEEIIIEKDFPIAPIYIYGNSYLFRNDKWTGWNTNFLERFDLCQLKLKNK

SEQ ID NO: 6 - protein, 166 aa, Borrelia burgdorferi
GLKGETKIILERSAKDITDEINKIKKDAADNNVNFAAFTDSETGSKVSENSFILEAKVRATTVAEKFVTAIEGEATKLKKTGSSGEFSAMYNMMLEVSGPLEELGVLRMTKTVTDAAEQHPTTTAEGILEIAKIMKTKLQRVHTKNYCALEKKKNPNFTDEKCKNN

SEQ ID NO: 7 - protein, 25 aa, synthetic construct
MKKDDQIAAAIALRGMAKDGKFAVK

SEQ ID NO: 8 - protein, 26 aa, synthetic construct
MKKDDQIAAAMVLRGMAKDGQFALKD

SEQ ID NO: 9 - protein, 26 aa, synthetic construct
MKKRNDKIVAAIVLRGVAKDGKFAAA

SEQ ID NO: 10 - protein, 25 aa, synthetic construct
MKKSDKIAAAIVLRGVAKDGKFAVA

SEQ ID NO: 11 - protein, 26 aa, synthetic construct
MKKRNDNIAAAIVLRGVAKSGKFAVA

SEQ ID NO: 12 - protein, 25 aa, synthetic construct
MKKDDQIAAAMVLRGMAKDGQFALK

SEQ ID NO: 13 - protein, 25 aa, synthetic construct
MKKSDKIAAAIVLRGVAKSGKFAVA

SEQ ID NO: 14 - protein, 25 aa, synthetic construct
MKKNDQIAAAIVLRGMAKDGEFALK

SEQ ID NO: 15 - protein, 26 aa, synthetic construct
MKKRNDNIAAAIVLRGVAKDGQFALK

SEQ ID NO: 16 - protein, 10 aa, synthetic construct
PVVAESPKKP

SEQ ID NO: 17 - protein, 14 aa, synthetic construct
CKWHVDNPIDEATA

SEQ ID NO: 18 - protein, 14 aa, synthetic construct
ITKLTPEELENLAK

SEQ ID NO: 19 - protein, 14 aa, synthetic construct
EKSKKEIEDQKNTK

SEQ ID NO: 20 - protein, 15 aa, synthetic construct
IFFLTFLCCNNKERK

SEQ ID NO: 21 - protein, 35 aa, synthetic construct
YGQNWTNPENMVTSGPFKLKERIPNEKIVFEKNNK

SEQ ID NO: 22 - protein, 15 aa, synthetic construct
SDYYSSAVNAIYFYS

SEQ ID NO: 23 - protein, 24 aa, synthetic construct
SDYYSSAVNAIYFYSFNTHIKPLD

SEQ ID NO: 24 - protein, 14 aa, synthetic construct
IYFYSFNTHIKPLD

SEQ ID NO: 25 - protein, 25 aa, synthetic construct
IYFYSFNTHIKPLDNVKIRKALTLA

SEQ ID NO: 26 - protein, 20 aa, synthetic construct
LAEAGYPNGNGFPILKLKYN

SEQ ID NO: 27 - protein, 20 aa, synthetic construct
KKICEFIQNQWKKNLNIDVE

SEQ ID NO: 28 - protein, 15 aa, synthetic construct
APIYIYGNSYLFRND

SEQ ID NO: 29 - protein, 25 aa, synthetic construct
NKTFNNLLKLTILVNLLISCGLTGA

SEQ ID NO: 30 - protein, 15 aa, synthetic construct
TILVNLLISCGLTGA

SEQ ID NO: 31 - protein, 22 aa, synthetic construct
PSESRAGNFILEAKVRATTVAE

SEQ ID NO: 32 - protein, 20 aa, synthetic construct
LVACSIGLVERTNAALESSS

SEQ ID NO: 33 - protein, 30 aa, synthetic construct
KDLKNKILKIKKEATGKGVLFEAFTGLKTG

SEQ ID NO: 34 - protein, 34 aa, synthetic construct
LQARILAVERYLKDQQLLGIWGCSGKLICTTTAP

SEQ ID NO: 35 - protein, 34 aa, synthetic construct
LQARVLAVERYLKDQKFLGLWGCSGKIICTTAAP

SEQ ID NO: 36 - protein, 32 aa, synthetic construct
LQARILAIERYLQDQQLLGIWGCSGKHICTTT

SEQ ID NO: 37 - protein, 69 aa, synthetic construct
SNNLDSKVGGNYNYLYRLFRKSNLKPFERDISTEIYQAGSTPCNGVEGFNCYFPLQSYGFQPTNGVGYQ

SEQ ID NO: 38 - protein, 24 aa, synthetic construct
LFRKSNLKPFERDISTEIYQAGST

SEQ ID NO: 39 - protein, 40 aa, synthetic construct
NGLTGTGVLTESNKKFLPFQQFGRDIADTTDAVRDPQTLE

SEQ ID NO: 40 - protein, 41 aa, synthetic construct
ITPCSFGGVSVITPGTNTSNQVAVLYQDVNCTEVPVAIHAD

SEQ ID NO: 41 - protein, 39 aa, synthetic construct
LYQDVNCTEVPVAIHADQLTPTWRVYSTGSNVFQTRAGC

SEQ ID NO: 42 - protein, 21 aa, synthetic construct
PVAIHADQLTPTWRVYSTGSN

SEQ ID NO: 43 - protein, 27 aa, synthetic construct
ASYQTQTNSPRRARSVASQSIIAYTMS

SEQ ID NO: 44 - protein, 28 aa, synthetic construct
KFQIYKTPPIKDGGFNFSQILPDPSKPS

SEQ ID NO: 45 - protein, 40 aa, synthetic construct
LPDPSKPSKRSFIEDLLFNKVTLADAGFIKQYGDCLGDIA

SEQ ID NO: 46 - protein, 40 aa, synthetic construct
AYKTFPPTEPKKDKKKKADETQALPQRQKKQQTVTLLPAA

SEQ ID NO: 47 - protein, 27 aa, synthetic construct
QRQKKQQTVTLLPAADLDDFSKQLQQS

SEQ ID NO: 48 - protein, 10 aa, synthetic construct
MNNQRKKARN

SEQ ID NO: 49 - protein, 10 aa, synthetic construct
NVLRGFRKEI

SEQ ID NO: 50 - protein, 10 aa, synthetic construct
MLNILNRRRR

SEQ ID NO: 51 - protein, 17 aa, synthetic construct
NNQRKKARNTPFNMLKR

SEQ ID NO: 52 - protein, 17 aa, synthetic construct
NVLRGFRKEIGRMLNIL

SEQ ID NO: 53 - protein, 17 aa, synthetic construct
KEIGRMLNILNRRRRTA

SEQ ID NO: 54 - protein, 17 aa, synthetic construct
ELCEDTMTYKCPRITEA

SEQ ID NO: 55 - protein, 17 aa, synthetic construct
AEPDDVDCWCNATDTWV

SEQ ID NO: 56 - protein, 18 aa, synthetic construct
QRVETWALRHPGFTVIAL

SEQ ID NO: 57 - protein, 10 aa, synthetic construct
IVSRQEKGKS

SEQ ID NO: 58 - protein, 8 aa, synthetic construct
KQNEPEDI

SEQ ID NO: 59 - protein, 16 aa, synthetic construct
KGKSLLFKTEDGVNMC

SEQ ID NO: 60 - protein, 17 aa, synthetic construct
HITEVEPEDIDCWCNLT

SEQ ID NO: 61 - protein, 14 aa, synthetic construct
TSTWVTYGTCNQAG

SEQ ID NO: 62 - protein, 18 aa, synthetic construct
LFKTASGINMCTLIAMDL

SEQ ID NO: 63 - protein, 12 aa, synthetic construct
DRGWGNGCGLFG

SEQ ID NO: 64 - protein, 9 aa, synthetic construct
RGARRMAIL

SEQ ID NO: 65 - protein, 18 aa, synthetic construct
KGMSYSMCTGKFKIVKEI

SEQ ID NO: 66 - protein, 18 aa, synthetic construct
RVQYEGDGSPCKIPFEIM

SEQ ID NO: 67 - protein, 18 aa, synthetic construct
GSPCKIPFEIMDLEKRHV

SEQ ID NO: 68 - protein, 24 aa, synthetic construct
RCIGISNRDFVEGVSGGSWVDIVL

SEQ ID NO: 69 - protein, 14 aa, synthetic construct
NTTTASRCPTQGEP

SEQ ID NO: 70 - protein, 26 aa, synthetic construct
MENKAWLVHRQWFLDLPLPWLPGADT

SEQ ID NO: 71 - protein, 21 aa, synthetic construct
KEIAETQHGTIVIRVQYEGDG

SEQ ID NO: 72 - protein, 19 aa, synthetic construct
WFKKGSSIGQMFETTMRGA

SEQ ID NO: 73 - protein, 15 aa, synthetic construct
RHVLGRLITVNPIVT

SEQ ID NO: 74 - protein, 15 aa, synthetic construct
EPGQLKLNWFKKGSS

SEQ ID NO: 75 - protein, 15 aa, synthetic construct
DSRCPTQGEAVLPEE

SEQ ID NO: 76 - protein, 15 aa, synthetic construct
TQGEAVLPEEQDPNY

SEQ ID NO: 77 - protein, 15 aa, synthetic construct
DSGCVVSWKNKELKC

SEQ ID NO: 78 - protein, 10 aa, synthetic construct
LTLNLITEMG

SEQ ID NO: 79 - protein, 10 aa, synthetic construct
PTFMTQKARD

SEQ ID NO: 80 - protein, 8 aa, synthetic construct
ATREAQKR

SEQ ID NO: 81 - protein, 12 aa, synthetic construct
FGQAAAGDKPPP

SEQ ID NO: 82 - protein, 12 aa, synthetic construct
FGQAAAGDKPAP

SEQ ID NO: 83 - protein, 12 aa, synthetic construct
FGQAAAGDKPSL

(19) United States
(12) Patent Application Publication
(10) Pub. No.: US 2025/0290925 A1
(43) Pub. Date: Sep. 18, 2025

(54) METHOD FOR DETECTING LYME DISEASE

(71) Applicant: Kephera Diagnostics, LLC, Framingham, MA (US)

(72) Inventors: Andrew E. Levin, Wellesley, MA (US); Nadezhda Karaseva, Wayland, MA (US)

(73) Assignee: Kephera Diagnostics, LLC, Framingham, MA (US)

(21) Appl. No.: 18/862,506

(22) PCT Filed: May 3, 2023
(86) PCT No.: PCT/US2023/066543

§ 371 Date: Nov. 1, 2024

Related U.S. Application Data
(60) Provisional application No. 63/338,374, filed on May 4, 2022.

(51) Int. Cl. G01N 33/569; G01N 33/543; G01N 33/68

(52) U.S. Cl. CPC G01N 33/56911; G01N 33/54388; G01N 33/6854; G01N 2333/20; G01N 2469/20

(57) ABSTRACT

Provided herein are methods, systems, microplates, compositions, and kits for detecting the presence or recent presence of an infection caused by an infectious disease in a subject. In some embodiments, the infectious disease is Lyme disease, hepatitis, Chagas disease, or a human immunodeficiency virus infection.

### CROSS-REFERENCE TO RELATED APPLICATIONS

This application claims the benefit under 35 U.S.C. § 119 (e) of U.S. Provisional Application No. 63/338,374, filed May 4, 2022, entitled “METHODS, SYSTEMS, MICROPLATES, USES, COMPOSITIONS, AND KITS FOR DETECTING INFECTIOUS DISEASES,” the entire disclosure of which is hereby incorporated by reference in its entirety.

### STATEMENT OF GOVERNMENT SUPPORT

This invention was made with Government support under Grant No. R43AI142903 awarded by the National Institute of Allergy and Infectious Disease (NIAID). The Government has certain rights in the invention.

### REFERENCE TO AN ELECTRONIC SEQUENCE LISTING

The contents of the electronic sequence listing (K071970000WO00-SEQ-KVC.xml; Size: 75,197 bytes; and Date of Creation: Apr. 28, 2023) is herein incorporated by reference in its entirety.

### FIELD OF THE INVENTION

The present disclosure relates to methods, systems, microplates, uses, compositions, and kits related to improved detection of infectious diseases.

### BACKGROUND

Infectious diseases including vector-borne diseases caused by microorganisms, such as bacteria, viruses, fungi, and parasites, account for over 4 million deaths per year in the United States alone. For example, Lyme disease has become the most prevalent vector-borne disease reported in the United States (Bacon et al., Morb Mortal Wkly Rep. 2008; 57 ((SS10)): 1-9.; Beard et al., U.S. Global Change Research Program, Washington, DC. 2016. p. 129-156). Although a total of about 30,000 cases are reported annually to the CDC, the CDC's estimates of the actual frequency of Lyme disease cases in the U.S. have risen in recent years from more than 300,000 per year (Nelson et al., Emerg Infect Dis. 2015; Hinckley et al., Clin Infect Dis. 2014; 59 (5): 676-81) to the most recent estimate of 476,000 per year (Kugeler et al., Emerg Infect Dis. 2021; 27 (2): 616-9). The economic impact of Lyme disease is likewise massive; recent studies estimate the annual healthcare cost at about $1 billion in the U.S., with overall economic losses up to $5 billion (Adrion et al., PLOS One. 2015 February; 10 (2): e0116767; Berry et al., Environ Resour Econ. 2017 April; 1-20). The spirochetal agent of Lyme disease, Borrelia burgdorferi (and related genospecies in Europe) is transmitted by the bite of Ixodes ticks, leading to a local dermatological infection which eventually spreads and disseminates to other tissues and organs. If undetected or untreated, it can become a chronic and debilitating illness. Early detection of infectious diseases, such as Lyme disease, has the potential to reduce the number of illnesses and/or deaths per year and improve outcomes. However, many existing detection methods are unable to identify an active or previous infection with specificity and sensitivity, resulting in false negatives and false positives, which may lead to worse outcomes, further spreading of infections, unnecessary treatments potentially leading to antibiotic resistance, and inefficient uses of health care systems.

### SUMMARY OF THE INVENTION

The present disclosure relates, at least in part, to methods, systems, microplates, compositions, kits, and uses for detection and diagnosis of the presence or recent presence of an infectious disease in a subject.

Aspects of the present disclosure relate to a method of detecting the presence of an antibody in a sample, the method comprising: (i) contacting the sample with a first antigen immobilized to a surface of a vessel and incubating the sample with the first antigen; (ii) contacting the sample with a second antigen conjugated to a detectable label and incubating the sample with the second antigen; and (iii) assessing the presence of a signal from the detectable label; wherein: the presence of a signal is indicative of association of the antibody with the first antigen and the second antigen; incubating the first antigen and the second antigen with the sample is sufficient to bind the first antigen to a first antigen-binding site on the antibody and the second antigen to a second antigen-binding site on the antibody, if present; and the first antigen and the second antigen are different.

Aspects of the present disclosure relate to a method of detecting the presence of an antibody in a sample, the method comprising: (i) adding the sample to a vessel comprising a surface coated with a first binding molecule; (ii) adding a first antigen conjugated to a second binding molecule to the vessel and incubating the sample with the first antigen; (iii) adding a second antigen conjugated to a detectable label to the vessel and incubating the sample with the second antigen; and (iv) assessing the presence of a signal from the detectable label; wherein: the presence of a signal is indicative of association of the antibody with the first antigen and the second antigen; incubating the first antigen and the second antigen with the sample is sufficient to bind the first antigen to a first antigen-binding site on the antibody and the second antigen to a second antigen-binding site on the antibody, if present; the first binding molecule binds to the second binding molecule; and the first antigen and the second antigen are different.

Aspects of the present disclosure relate to a method of detecting the presence of an antibody in a sample, the method comprising: (i) incubating the sample with a first antigen to form an antibody-first antigen complex, wherein the first antigen is bound to a first binding molecule; (ii) immobilizing a second antigen on a solid substrate; (iii) adding the sample comprising the antibody-first antigen complex to the solid substrate; (iv) incubating the solid substrate comprising the sample comprising the antibody-first antigen complex for a sufficient time to further bind the antibody-first antigen complex to the second antigen to form a second antigen-antibody-first antigen complex; (v) adding a second binding molecule conjugated to a detectable label to the solid substrate; and (vi) assessing the presence of a signal from the detectable label; wherein: the presence of a signal is indicative of association of both (1) the antibody with the first antigen and the second antigen and (2) the first binding molecule with the second binding molecule; incubating the sample with the first antigen is sufficient to bind the first antigen to a first antigen-binding site on the antibody, if present; incubating the sample with the second antigen immobilized on the solid substrate is sufficient to bind the second antigen to a second antigen-binding site on the antibody, if present; and the first antigen and the second antigen are different.

Aspects of the present disclosure relate to a method of detecting the presence of an antibody in a sample, the method comprising: (i) immobilizing a first antigen on a solid substrate; (ii) adding a second antigen to the solid substrate, wherein the second antigen is conjugated to a detectable label or is adapted to be conjugated to the detectable label; (iii) adding the sample to the solid substrate; (iv) incubating the solid substrate comprising the sample, the first antigen and the second antigen for sufficient time to bind the second antigen to a first antigen-binding site on the antibody in the sample, if present; (v) incubating the solid substrate comprising the sample, the first antigen and the second antigen for sufficient time to bind the first antigen to a second antigen-binding site on the antibody in the sample, if present; and (vi) assessing the presence of a signal from the detectable label; wherein: the presence of a signal is indicative of association the antibody with the first antigen and the second antigen; and the first antigen and the second antigen are different.

Aspects of the present disclosure relate to a system comprising: a microplate comprising a plurality of wells; and a first antigen immobilized or adapted to be immobilized to at least one of the plurality of wells; wherein: at least one of the plurality of wells including the first antigen is adapted to receive: (i) a sample to be analyzed for the presence of an antibody; and (ii) a second antigen conjugated to a detectable label; wherein: the first antigen binds to a first antigen-binding site on the antibody and the second antigen binds to a second antigen-binding site on the antibody, if present; and the first antigen and the second antigen are different.

Aspects of the present disclosure relate to a microplate, comprising: a plurality of wells; and a first antigen immobilized to a surface of at least one of the plurality of wells; wherein: the microplate is adapted to receive: a sample to be analyzed for the presence of an antibody; and a second antigen conjugated to a detectable label in at least one of the plurality of wells including the first antigen; the first antigen binds to a first antigen-binding site on the antibody and the second antigen binds to a second antigen-binding site on the antibody, if present; and the first antigen and the second antigen are different.

Aspects of the present disclosure relate to a microplate, comprising: a plurality of wells; a first antigen immobilized to a surface of at least one of the plurality of wells; a second antigen disposed in at least one of the plurality of wells including the first antigen; and an antibody bound to the first antigen and the second antigen; wherein: the second antigen is conjugated to a detectable label; and the first antigen and the second antigen are different.

Aspects of the present disclosure relate to a kit, comprising: a microplate comprising a plurality of wells and a first antigen immobilized to a surface of at least one of the plurality of wells; and a reagent comprising a second antigen conjugated to a detectable label; wherein: the microplate is adapted to receive a sample to be analyzed for the presence of an antibody; and the first antigen and the second antigen are different.

Aspects of the present disclosure relate to a kit, comprising: a microplate comprising a plurality of wells, wherein the plurality of wells are coated with a first binding molecule; a reagent comprising a first antigen conjugated to a second binding molecule; and a reagent comprising a second antigen conjugated to a detectable label; wherein: the microplate is adapted to receive a sample to be analyzed for the presence of an antibody; and the first antigen and the second antigen are different.

Aspects of the present disclosure relate to a kit, comprising: a solid substrate; a reagent comprising a first antigen conjugated to a first binding molecule; a reagent comprising a second antigen; and a reagent comprising a second binding molecule conjugated to a detectable label; wherein: the substrate is adapted to receive a sample to be analyzed for the presence of an antibody; and the first antigen and the second antigen are different.

Aspects of the present disclosure relate to a composition, comprising: an antibody derived from a sample; a first antigen bound to a first antigen-binding site; a second antigen bound to a second antigen-binding site; wherein: the second antigen is conjugated to a detectable label; and the first antigen and the second antigen are different.

Aspects of the present disclosure relate to a one-step method of detecting the presence of an antibody in a sample, the method comprising: (i) contacting the sample with a first antigen immobilized to a surface of a vessel and incubating the sample with the first antigen; (ii) contacting the sample with a second antigen conjugated to a detectable label and incubating the sample with the second antigen; and (iii) assessing the presence of a signal from the detectable label; wherein: the presence of a signal is indicative of association of the antibody with the first antigen and the second antigen; incubating the first antigen and the second antigen with the sample is sufficient to bind the first antigen to a first antigen-binding site on the antibody and the second antigen to a second antigen-binding site on the antibody, if present; the first antigen and the second antigen are different; and the method does not require a confirmatory test.

In some embodiments, the first antigen is a recombinant protein. In some embodiments, the second antigen is a peptide derived from the recombinant protein. In some embodiments, the second antigen is a recombinant protein. In some embodiments, the first antigen is a peptide derived from the recombinant protein. In some embodiments, the first antigen is selected from a group consisting of: VlsE, C6, OspC, C10, BBK07, OppA2, and Decorin Binding Protein A. In some embodiments, the second antigen is selected from a group consisting of: VlsE, C6, OspC, C10, BBK07, OppA2, and Decorin Binding Protein A. In some embodiments, the first antigen is VlsE and the second antigen is C6. In some embodiments, the first antigen is C6 and the second antigen is VlsE. In some embodiments, the first antigen is OspC and the second antigen is C10. In some embodiments, the first antigen is C10 and the second antigen is OspC.

In some embodiments, the first binding molecule is an avidin protein and the second binding molecule is a biotin. In some embodiments, the first binding molecule is a biotin and the second binding molecules is an avidin protein. In some embodiments, the avidin protein is NeutrAvidin or streptavidin. In some embodiments, the avidin protein is streptavidin.

In some embodiments, the sample is a biological sample. In some embodiments, the biological sample is selected from the group consisting of: saliva, oral fluid, tears, urine, interstitial fluid, synovial fluid, and cerebrospinal fluid. In some embodiments, the biological sample is blood. In some embodiments, the biological sample is blood, serum, plasma, or a blood fraction. In some embodiments, the blood is collected from a subject suspected of having a Lyme disease infection. In some embodiments, the subject is a human or a non-human animal. In some embodiments, the subject is a human.

In some embodiments, the sample is collected from a subject suspected of having a disease caused by a pathogenic member of the bacterial genus Borrelia. In some embodiments, the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica. In some embodiments, the pathogenic member of the bacterial genus Borrelia is Borrelia burgdorferi.

In some embodiments, the first antigen is a peptide or protein comprising an amino acid sequence having at least 90% sequence identity to the amino acid sequence set forth in any one of SEQ ID NOs: 1-83. In some embodiments, the first antigen is a protein comprising an amino acid sequence having at least 90% sequence identity to the amino acid sequence set forth in any one of SEQ ID NO: 1-6. In some embodiments, the second antigen is a peptide or protein comprising an amino acid sequence having at least 90% sequence identity to the amino acid sequence set forth in any one of SEQ ID NOs: 1-83. In some embodiments, the second antigen is a peptide comprising an amino acid sequence having at least 90% sequence identity to the amino acid sequence set forth in any one of SEQ ID NOS: 7-83. In some embodiments, the sequence identity is at least 91%, at least 92%, at least 93%, at least 94%, at least 95%, at least 96%, at least 97%, at least 98%, at least 99%, at least 99.5%, or at least 100%.

In some embodiments, the method further comprises a wash step in between step (ii) and step (iii). In some embodiments, the wash step comprises adding a wash buffer to the vessel and incubating the wash buffer in the vessel for a sufficient time to remove unbound antibodies.

In some embodiments, the detectable label is a luminescent label, a colorimetric label, or a radiometric label. In some embodiments, the luminescent label is a luminescent dye, a FRET label, or a fluorescent protein. In some embodiments, the luminescent label is a lanthanide, a fluorescent label, or an organic dye. In some embodiments, the colorimetric label is a horseradish peroxidase label or alkaline phosphatase. In some embodiments, the method further comprises a step of detecting the detectable label between step (ii) and step (iii). In some embodiments, the step of assessing the presence of a signal comprises detecting signal pulses from the microplate wells. In some embodiments, the detectable label is adapted to be detected by detecting a signal pulse emitted by the detectable label. In some embodiments, the step of detecting comprises adding a substrate to the microplate wells. In some embodiments, the detectable label is adapted to be detected by a substrate. In some embodiments, the substrate is selected from the group consisting of: ABTS, OPD, AmplexRed, DAB, AEC, TMB, Homovanillic Acid, or Luminol. In some embodiments, the substrate is 3,3′,5,5′-tetramethylbenzidine (TMB). In some embodiments, the substrate is Luminol.

In some embodiments, the vessel is adapted to receive at least a portion of the sample. In some embodiments, the vessel is a well. In some embodiments, the vessel is a well of a microplate comprising one or more wells. In some embodiments, the presence of a signal is also indicative of the presence of the antibody in the sample. In some embodiments, the presence of a signal is suggestive of a Lyme disease infection in the subject. In some embodiments, the presence of a signal is indicative of Lyme disease-specific antibodies. In some embodiments, the presence of a signal is indicative of a previous or active Lyme disease infection in the subject. In some embodiments, the presence of a signal is indicative of an active Lyme disease infection in the subject.

In some embodiments, the method further comprises administering an antibiotic to the subject from whom the sample was obtained. In some embodiments, the antibiotic is selected from the group consisting of: amoxicillin, cefuroxime, doxycycline, and ceftriaxone. In some embodiments, the antibiotic is doxycycline.

In some embodiments, steps (i) and (ii) are performed simultaneously. In some embodiments, steps (ii) and (iii) are performed simultaneously.

In some embodiments, the first antigen-binding site and the second antigen-binding site are identical. In some embodiments, the first antigen-binding site and the second antigen-binding site share at least 90% sequence identity. In some embodiments, the first antigen-binding site and the second antigen-binding site share 91%, 92%, 93%, 94%, 95%, 96%, 97%, 98%, 99%, 99.5%, or 100% sequence identity. In some embodiments, the first antigen is capable of binding to the first antigen-binding site or the second antigen-binding site. In some embodiments, the second antigen is capable of binding to the first antigen-binding site or the second antigen-binding site.

In some embodiments, step (i) is performed before step (ii). In some embodiments, step (ii) is performed before step (i). In some embodiments, step (ii) is performed before step (iii). In some embodiments, step (iii) is performed before step (ii).

In some embodiments, the first antigen is a first peptide and the second antigen is a second peptide. In some embodiments, the method does not require a confirmatory test.

In some embodiments, the disease to be detected is an infectious disease. In some embodiments, the disease or infectious agent to be detected is selected from the group consisting of: Lyme disease, hepatitis, Chagas disease, and a human immunodeficiency virus infection. In some embodiments, the first antigen is bound to a complex. In some embodiments, the second antigen is bound to a complex. In some embodiments, the conjugate is selected from the group consisting of: horseradish peroxidase (HRP), biotin, an avidin protein, a maleimide-activated HRP, or a maleimide.

In some embodiments, the solid substrate is a membrane. In some embodiments, the membrane is a nitrocellulose membrane. In some embodiments, the membrane is a Fusion 5 membrane. In some embodiments, the first antigen is a peptide comprising an amino acid sequence having at least 90% sequence identity to the amino acid sequence set forth in any one of SEQ ID NOs: 7-83. In some embodiments, the second antigen is a protein comprising an amino acid sequence having at least 90% sequence identity to the amino acid sequence set forth in any one of SEQ ID NOs: 1-6.

In some embodiments, the steps of the method are performed sequentially. In some embodiments, the steps of the method are performed simultaneously. In some embodiments, the steps of the method are performed in any order. In some embodiments, the detectable label is conjugated to the first antigen. In some embodiments, the detectable label is conjugated to the second antigen. In some embodiments, the detectable label is conjugated to the first binding molecule. In some embodiments, the detectable label is conjugated to the second binding molecule.

In some embodiments, the first antigen is immobilized directly on the solid substrate. In some embodiments, the first antigen is immobilized to the solid substrate via a binding molecule pair. In some embodiments, the detectable label is conjugated directly to the second antigen. In some embodiments, the detectable label is conjugated to the second antigen via a binding molecule pair. In some embodiments, the binding molecule pair comprises a first binding molecule and a second binding molecule. In some embodiments, the method further comprises the step of conjugating the detectable label to the second antigen.

The details of one or more embodiments of the present disclosure are set forth in the description below. Other features or advantages of the present disclosure will be apparent from the following drawings and detailed description of several embodiments, and also from the appended claims.

### BRIEF DESCRIPTION OF THE DRAWINGS

The accompanying drawings are not necessarily intended to be drawn to scale. For purposes of clarity, not every component may be labeled in every drawing. In the drawings:

FIG. 1 shows a schematic of an exemplary bridge assay for a primary antibody as described herein.

FIG. 2 shows a schematic of an exemplary Hybrid ELISA for a serum antibody including a VlsE protein, and a C6 peptide/streptavidin-horseradish peroxidase conjugate as described herein.

FIG. 3 shows an exemplary Hybrid ELISA procedure as described herein.

FIG. 4 shows a schematic of steps for an exemplary Hybrid ELISA as described herein.

FIGS. 5A-5C show the absorbance distribution (FIG. 5A), ROC curves (FIG. 5B), sensitivity and specificity for 76 confirmed Lyme positive and 100 healthy control sera tested on Hybrid ELISA and VlsE ELISA (FIG. 5C) as described herein.

FIGS. 6A-6B show the absorbance distribution and ROC curve for 113 confirmed Lyme positive sera and 486 negative control sera as described herein.

FIG. 7 shows the detection of early Lyme sera vs. days after onset of symptoms as described herein. Detection by Hybrid ELISA (left columns) vs. C6 ELISA (right columns) was compared.

FIG. 8 shows the analytical sensitivity of Hybrid ELISA vs. C6 peptide indirect ELISA as described herein.

FIG. 9 shows a plot of sample dilution factor vs. Lyme index value, demonstrating the Hook Effect in a Hybrid ELISA as described herein.

FIG. 10 shows data from accelerated stability testing of Hybrid ELISA as described herein.

FIG. 11 shows a schematic of a Hybrid Lateral Flow Immunoassay strip as described herein.

FIG. 12 shows a photograph of results from an exemplary Hybrid Lateral Flow Immunoassay as described herein.

FIG. 13 shows a photograph of a scoring guide for an exemplary Hybrid Lateral Flow Immunoassay read guide as described herein.

FIG. 14 shows results from an exemplary Hybrid Lateral Flow Immunoassay as described herein.

FIG. 15 shows a schematic of an exemplary C6-VlsE Hybrid Lateral Flow Immunoassay schematic as described herein.

### DETAILED DESCRIPTION

Lyme disease, a bacterial zoonosis which is now the most common vector-borne disease in the United States, has continued to pose a diagnostic challenge to clinicians and a threat of chronic illness to patients. The threat to public health associated with Lyme disease has prompted the development of several vaccines against Lyme disease (Marques et al., J Clin Microbiol. 2002 July; 40 (7): 2591-2593; Comstedt et al., PloS One. 2017; 12 (9)). One vaccine against Lyme disease recently entered Phase 3 clinical trials; however, improved diagnostics are needed to support effective use of a possible vaccine by clinicians (Czarnota et al., Journal of Education, Health and Sport. 2022 August; 12 (8): 1164-1169). Specifically, improved diagnostics are needed to support effective use of a Lyme disease vaccine by clinicians due to the potential for vaccine-induced seroreactivity. Seroreactivity could result in false positive results in vaccinated individuals due to cross-reactivity of vaccine antigens with the antigens employed in some currently used Lyme disease assays. For example, OspA Lyme disease vaccines include an OspA antigen which is also a natural component of the Lyme spirochete (B. burgdorferi), and which would be present in whole cell sonicates that have been used in a variety of Lyme ELISA assays.

The overlap in symptoms between Lyme disease and a variety of other conditions underlies the difficulty in clinical diagnosis. The only uniquely identifiable clinical symptom of infection is erythema migrans (EM), the circular bull's eye rash that occurs within days or weeks of infection, but is absent, unnoticed or misidentified in a significant fraction of cases (Aucott et al., BMC Infect Dis. 2009 December; 9 (1): 79; Aucott et al., Dermatol Res Pract. 2012 October; 2012:1-6; Moore et al., Emerg Infect Dis. 2016 July; 22 (7): 1169-77). Hence diagnosis in most cases rests on the results of laboratory testing. Serology is the mainstay of diagnostic testing as spirochetes rapidly disappear from the blood after infection. This is hampered, however, by the varying sensitivity and specificity of available serological tests. Given the widespread testing of individuals at low risk for Lyme disease—either due to location in a non-endemic area or absence of other risk factors or symptoms—the result is that a positive screening result is more likely to be false than true based on predictive value. A two-step serological approach is therefore recommended by Centers for Disease Control and Prevention (“CDC”), comprising an enzyme-linked immunosorbent assay (“ELISA”) or other screening assay followed by IgG and IgM immunoblots to confirm a positive result (CDC, MMWR Morb Mortal Wkly Rep. 1995; 44 (31): 590-1). This serial two-tier testing protocol increases overall specificity, and has effectively reduced the rate of false positivity for IgG, at least, to about 0.5%. However, the two-tier protocol has been shown to have low sensitivity for early Lyme disease, in the 30-40% range, due primarily to the high stringency and low sensitivity of the immunoblot (Branda et al., Clin Infect Dis. 2018; 66 (7): 1133-9; Theel et al., J Clin Microbiol. 2016; 54 (5): 1191-6); it also extends the turnaround time and adds cost and logistical complexity to the testing process. Recently studies have shown that the immunoblot can be substituted by a second ELISA with a gain in overall sensitivity but without significant loss in specificity; this represents a significant step forward but does not change the requirement for a two-tier testing protocol.

The hallmark symptom of Lyme disease—EM—was first described by Afzelius over a century ago (Afzelius et al., Arch Dermatol Syph. 1910; 101:403-6), and the arthritic manifestations of Lyme disease have been recognized for years, however Lyme disease often presents with more non-specific symptoms, including flu-like illness and arthralgia. In about 30-40% of cases, erythema migrans is not observed and/or the subject has no memory of a tick bite (Moore et al., Emerg Infect Dis. 2016 July; 22 (7): 1169-77; Wormser et al., Diagn Microbiol Infect Dis. 2013 January; 75 (1): 9-15). Moreover, the full spectrum of Lyme disease symptoms overlaps with numerous other disease conditions, including chronic fatigue, fibromyalgia, rheumatoid arthritis, multiple sclerosis, Parkinson's disease, Guillain-Barre syndrome, ALS, and others (Borgermans et al., Int J Family Med. 2014; 2014:138016). For these reasons, diagnosis of Lyme disease can be challenging. Although the diagnosis can be made based on the identification of an EM rash, in the many cases where the rash is not apparent, cannot be clearly distinguished from other skin lesions, or where the disease has progressed beyond the EM stage, diagnosis currently relies on a two-step serological approach recommended by the CDC since 1994 (CDC, MMWR. 1995; 44 (31): 590-1). In the original “standard” two-tier testing (“STTT”) approach, serum specimens are tested first by ELISA, and those found positive or indeterminate are subsequently tested by IgG and IgM immunoblot assays. This two-tier testing protocol was approved in response to the relatively high false positive rate documented for ELISAs at the time. While ELISA is relatively easy to perform, with a typical turnaround time of several hours, allowing same day or overnight results, immunoblot is a specialty technique that is not widely available and requires a send-out, with a wait of up to several days. Consequently, the clinician can be forced to choose between prescribing antibiotics before the test result is confirmed and accepting the possibility of a negative result and attendant inappropriate antibiotic usage, or delaying the initiation of treatment until test results are available. Neither alternative is desirable from the point of view of medical efficacy, health outcomes or cost-efficiency, and the rate of error by clinicians in diagnosing Lyme disease in advance of test results has been found high in both directions (Nigrovic et al., Pediatrics. 2017 December; 140 (6): e20171975). The unnecessary prescription of antibiotics contributes to the rise in antibiotic resistance among many bacterial pathogens.

Few infectious disease tests require a second, confirmatory assay. For HIV, the requirement was based on the combination of inadequate specificity of early screening assays and the lethal implications of a positive diagnosis. Other diseases requiring confirmatory assays fit a similar profile, including hepatitis and Chagas disease. Lyme disease, by contrast, is debilitating but rarely if ever lethal. The rationale for requiring a confirmatory assay for Lyme is based on two factors—the still suboptimal specificity of most serologic tests and the fact that the vast majority of Lyme testing is carried out on low-risk individuals, notwithstanding public health guidelines, decreasing the positive predictive value of the test. As evidence for the latter, CDC has reported that over 3,000,000 Lyme tests are carried out annually in the U.S., but the number of true cases—both tested and untested—is estimated at ˜500,000, so that >80% of tests are performed on individuals with no Lyme disease (Nelson et al., Emerg Infect Dis. 2015; Hinckley et al., Clin Infect Dis. 2014; 59 (5): 676-81). Given the likelihood that this pattern will continue, minimizing the frequency of false positive results is dependent on maintaining or exceeding the high assay specificity of the two-tier serial testing algorithm.

A further complication is that the sensitivity of the two-tier method is as low as 30%-40% in early Lyme disease, principally due to the relatively insensitive immunoblot step, which leads to false negative results (Moore et al., Emerg Infect Dis. 2016 July; 22 (7): 1169-77; Waddell et al., PLOS One. 2016 Dec. 21]; 11 (12): e0168613; Branda et al., Clin Infect Dis. 2017; 64 (8): 1074-80; Branda et al., Clin Infect Dis. 2011). A succession of published studies showed that replacement of the immunoblot with a second ELISA in a serial two-test algorithm resulted in higher sensitivity without a significant decrease in specificity, particularly in early Lyme disease; this approach has been termed the “modified two-tier testing” or “MTTT” algorithm (Branda et al., Clin Infect Dis. 2017; 64 (8): 1074-80; Pegalajar-Jurado et al., J Clin Microbiol. 2018 Aug. 1; 56 (8): e01943-17). The initial test is based on the ELISA assay described above and the second test is an ELISA based on the C6 peptide. The C6 peptide ELISA was based on an immunodominant 26 amino acid peptide which forms the 6^{th} invariant region of the B. burgdorferi surface protein VlsE, and was the first peptide-based ELISA for Lyme disease to show performance equal or better than that of conventional whole cell lysate-derived ELISAs (Ting et al., J Clin Microbiol. 1999; 37 (12): 3990-6) Wormser et al., Diagn Microbiol Infect Dis. 2013 January; 75 (1): 9-15), but fell short of the 99.5% specificity reported for standardized two-tier testing. The roughly 1-1.5% lower specificity of the C6 ELISA would translate to ≥10,000 false positive results per million tests, which was considered by many clinicians and by the FDA as unacceptable, precluding use of the C6 ELISA as a single step test. As no Lyme disease ELISA to date has shown specificity equal to that of two-tier testing, the two-tier testing algorithm continues to be the only acceptable approach to deliver acceptable performance.

Conventional Lyme serological assays are based on an indirect ELISA format where serum antibodies bind B. burgdorferi antigen(s) immobilized on a solid substrate (e.g. the wells of a microplate). Bound antibodies are then detected by addition of a second, labeled reagent (e.g., anti-human IgG-horseradish peroxidase (HRP) conjugate). This conventional sandwich assay design offers the advantages that commercial off-the-shelf reagents can be used for the detection step, and that specificity for a given antibody class (e.g., IgG or IgM) and host species can be built into the assay. However, these advantages are offset by a significant disadvantage—the non-specific binding inherent to antibody conjugates, which leads to a level of non-specific reactivity and false positivity (often 1% or more (Lantos et al., Clin Infect Dis. 2015 November; 61 (9): 1374-80; Gregson et al., CMAJ. 2015 November; 187 (16): 1193-4; Fallon et al., Clin Infect Dis. 2014 December; 59 (12): 1705-10) which degrades assay performance. In the context of Lyme disease, where more than 3 million tests are performed annually in the U.S., a 1% false positive rate would translate to 30,000 false positive diagnoses. In addition to the unnecessary prescription of antibiotics, such false positive diagnoses may divert attention away from the true cause of illness and delay needed treatment.

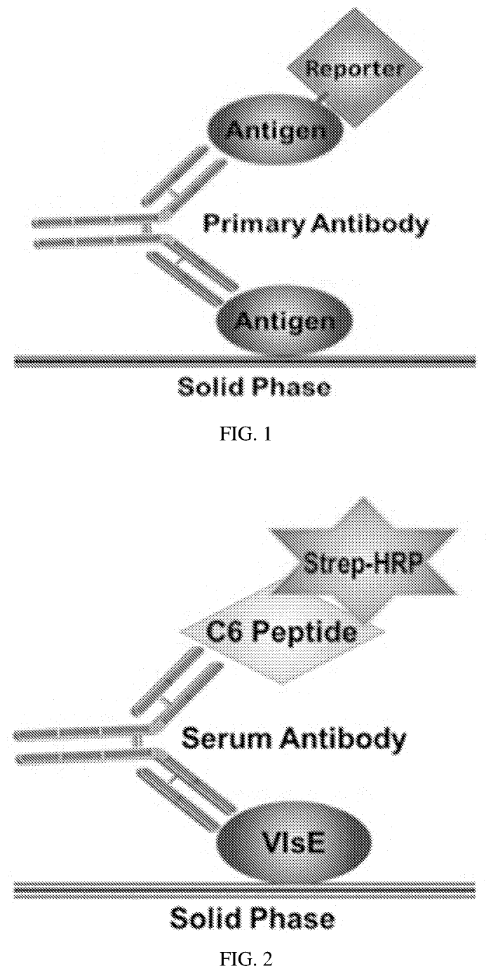

The inventors have unexpectedly solved problems presented by both the classic ELISA followed up immunoblot testing regime and the MTTT testing regime by developing a novel assay design, termed “Hybrid ELISA.” Without wishing to be bound by theory, the inventors posit that a subset of antibodies is present in sera from true infectious disease (e.g., Lyme disease, HIV, Chagas disease, Dengue, and other viral, bacterial, or parasitic diseases) subjects which recognizes both the linear epitope in a peptide or protein derived from the native infectious protein (e.g., for Lyme disease, the isolated C6 peptide or the isolated C10 peptide) and the same epitope when present in its native conformation (e.g., as part of the VlsE protein, the OspC protein, the BBK07 protein, the OppA2 protein, or the Decorin Binding Protein A). The extent to which the two epitope presentations may differ antigenically based on conformational constraints imposed by the intact protein is not known, but was suggested by one study in the context of Lyme disease (Embers et al., Clin Vaccine Immunol. 2007). The inventors posit that antibodies which bind one or another antigen non-specifically recognize some features of the antigen that are distinct from those recognized by truly specific antibodies, and that such non-specific features might not be shared between the linear peptide and the native protein conformations. The inventors further posit that the same antibody molecule could bind both the linear peptide and the native protein in a bridge assay utilizing both the native protein and its derived peptide version as antigens (e.g., in which C6 peptide is one of the antigens and VlsE is the other, as shown in FIG. 2), yielding high sensitivity but more importantly-exceptionally high specificity. The inventors believe that this approach has no precedent and the results described herein (e.g., for Lyme disease) are the first demonstration of its feasibility. The inventors further posit that same approach would presumably be similarly effective in detecting antibodies directed against other pathogen and non-pathogen antigens, where both a native or recombinant protein antigen and a peptide derived from it are known to be antigenic. Examples can be found in the immunodominant IDR peptide derived from the HIV-1 gp41 antigen (Target product profile, 2017); peptides derived from the TcCA-2 antigen of Trypanosoma cruzi, the agent of Chagas disease (Thomas et al., Clin Vaccine Immunol. 2012; 19 (2): 167-73), and multiple immunodominant peptides derived from Dengue and other flaviviruses (Fumagalli et al., Front Cell Infect Microbiol. 2021; 11 (August): 1-16). Other examples include OspC and C10, BBK07, OppA2, DbpA, DbpB, and the SARS-CoV-2 spike protein.

The present disclosure accordingly relates to a novel ELISA methodology for infectious disease antibody detection based on the unique discovery by the inventors that enables exceptionally high assay specificity. In particular, the inventors of the present disclosure have developed, based on the surprising and unexpected discoveries described herein, a novel immunochemical methodology on which the Hybrid ELISA is based. In one embodiment, the test is a Lyme disease diagnostic based on the proven, highly sensitive C6 peptide and VlsE protein antigens in an immunochemical format. VlsE (Variable Large Protein) is a lipoprotein located on the surface of the Lyme Disease spirochete. C6 is a peptide derived from the VlsE protein. Each of the C6 peptide and VlsE protein antigens have been shown in multiple studies to offer both high sensitivity and high specificity in detection of Lyme disease, covering almost all known pathogenic Borrelia strains causing Lyme disease, including those in the U.S. and Borrelia burgdorferi sensu lato strains B. afzelii and B. garinii in Europe, and more recently B. mayonii (Branda et al., Clin Infect Dis. 2017; 64 (8): 1074-80; Wormser et al., Diagn Microbiol Infect Dis. 2013 January; 75 (1): 9-15; Zhang et al., Cell. 1997; 89 (2): 275-85; Lawrenz et al., J Med Microbiol. 2002 August; 51 (8): 649-55; Lawrenz et al., J Clin Microbiol. 1999 December; 37 (12): 3997-4004; Pritt et al., Lancet Infect Dis. 2016 May; 16 (5): 556-64; Ting et al., J Clin Microbiol. 1999; 37 (12): 3990-6; Liang et al., J Infect Dis. 2000; 182 (5): 1455-62; Bacon et al., J Infect Dis. 2003; 187 (8): 1187-99; Wormser et al., Clin Infect Dis. 2008 October; 47 (7): 910-4; Wormser et al., Clin Vaccine Immunol. 2008; 15 (10): 1519-22; Branda et al., Clin Infect Dis. 2013; Wormser et al., Med Microbiol Immunol. 2014; Lipsett et al., Clin Infect Dis. 2016; 63 (1 October): 922-8). The novel assay relies, in some embodiments, on the simultaneous binding of similar epitopes (i.e., the parts of an antigen molecule to which an antibody attaches) present on two different molecules—the C6 synthetic peptide and the VlsE recombinant protein from which C6 is derived—by individual antibody molecules in patient sera. The inventors have unexpectedly discovered that this requirement builds additional specificity into the assay, eliminating the vast majority of non-specific antibody interactions responsible for false positive results. The Hybrid ELISA disclosed herein accordingly provides a result equivalent in accuracy to that yielded by conventional two-tier testing methods, but in a single tier assay. The inventors believe that removing the requirement for immunoblot confirmation will double the sensitivity of serologic detection in early stage Lyme disease (Wormser et al., Diagn Microbiol Infect Dis. 2013 January; 75 (1): 9-15) without compromising its specificity.

Aspects of the present disclosure relate to detection and diagnosis of the presence or recent presence of Lyme disease or another infectious disease in a subject. In some embodiments, the present disclosure relates to collecting a sample from a subject and exposing said sample to the Hybrid ELISA assay described herein.

Aspects of the present disclosure relate to the use of a unique combination of proteins, peptides, or proteins and peptides in an assay to detect or diagnose the presence or recent presence of Lyme disease or another infectious disease in a subject. In some embodiments, the assay is a Hybrid ELISA assay, as described herein. In some embodiments, the assay is a lateral flow assay. In some embodiments, the assay is an assay that uses two proteins and/or peptides to bind a target molecule. In some embodiments, a target molecule is an antibody.

Aspects of the present disclosure relate to a method of detecting the presence of an antibody in a sample, the method comprising: contacting the sample with a first antigen immobilized to a surface of a vessel and incubating the sample with the first antigen; contacting the sample with a second antigen conjugated to a detectable label and incubating the sample with the second antigen; and assessing the presence of a signal from the detectable label; wherein the presence of a signal is indicative of association of the antibody with the first antigen and the second antigen; incubating the first antigen and the second antigen with the sample is sufficient to bind the first antigen to a first antigen-binding site on the antibody and the second antigen to a second antigen-binding site on the antibody, if present; and the first antigen and the second antigen are different. In some embodiments, the methods, systems, microplates, kits, compositions, uses, and tests described herein can be used to detect any infectious disease. In some embodiments, the infectious disease is Lyme disease, hepatitis, Chagas disease, human immunodeficiency virus, or a coronavirus (e.g., severe acute respiratory syndrome coronavirus 1 (SARS-CoV-1), severe acute respiratory syndrome coronavirus 2 (SARS-CoV-2)).

In some embodiments, the method is a one-step test for detection of infectious disease. In some embodiments, the method does not require a confirmatory test. In some embodiments, each of the steps described herein are conducted in order. In some embodiments, each of the steps described herein are not conducted in order. In some embodiments, each of the steps described herein are conducted simultaneously. In some embodiments, a portion of the steps described herein are conducted simultaneously. In some embodiments, the steps of contacting the first and second antigens with an antibody are conducted consecutively. In some embodiments, the steps of contacting the first and second antigens with an antibody are conducted simultaneously.

In some embodiments, the first antigen is a recombinant protein. In some embodiments, the second antigen is a peptide derived from the recombinant protein. In some embodiments, the second antigen is a recombinant protein. In some embodiments, the first antigen is a peptide derived from the recombinant protein. In some embodiments, the first antigen is a first peptide and the second antigen is a second peptide.

In some embodiments, the first antigen is selected from a group consisting of: VlsE, C6, OspC, C10, BBK07, OppA2, and Decorin Binding Protein A. In some embodiments, the second antigen is selected from a group consisting of: VlsE, C6, OspC, C10, BBK07, OppA2, and Decorin Binding Protein A. In some embodiments, the first antigen is VlsE and the second antigen is C6. In some embodiments, the first antigen is C6 and the second antigen is VlsE. In some embodiments, the first antigen is OspC and the second antigen is C10. In some embodiments, the first antigen is C10 and the second antigen is OspC.

In some embodiments, the amino acid sequence of VlsE is derived from the B31 strain of Borrelia burgdorferi. A non-limiting example of the amino acid sequence of VlsE is accessible through NCBI GenBank Accession No. AAC45733.1, and is provided here as SEQ ID NO: 1:

MKSQVADKDDPTNKFYQSVIQLGNGFLDVFTSFGGLVAEAFGFKSDPKK
SDVKTYFTTVAAKLEKTKTDLNSLPKEKSDISSTTGKPDSTGSVGTAVE
GAIKEVSELLDKLVKAVKTAEGASSGTAAIGEVVADADAAKVADKASVK
GIAKGIKEIVEAAGGSEKLKAVAAAKGENNKGAGKLFGKAGAAAHGDSE
AASKAAGAVSAVSGEQILSAIVTAADAAEQDGKKPEEAKNPIAAAIGDK
DGGAEFGQDEMKKDDQIAAAIALRGMAKDGKFAVKDGEKEKAEGAIKGA
AESAVRKVLGAITGLIGDAVSSGLRKVGDSVKAASKETPPALNK

In some embodiments, SEQ ID NO: 1 or any other sequence described herein further comprises a histidine tag at the N-terminus. The term “histidine tag” or “polyhistidine,” as used interchangeably and used herein, refers to an amino acid sequence comprising at least six histidine residues. In some embodiments, a histidine tag is at least 6, at least 7, at least 8, at least 9, or at least 10 amino acid residues in length. In some embodiments, a histidine tag is useful for protein purification and other laboratory techniques, as will be understood by a skill artisan. In some embodiments, SEQ ID NO: 1 or any other sequence described herein further comprises a signal peptide. The term “signal peptide,” as used herein, refers to a short peptide, typically at the N-terminus of an amino acid sequence, that facilitates transport of said amino acid sequence to a location within or outside of a cell.

A non-limiting example of the amino acid sequence of VlsE is accessible through UniProtKB Accession No. Q5DVG3, and is provided here as SEQ ID NO: 2:

MNTKKISSAILLTTFFVFINCKSQVADKDDPTNKFYQSVIQLGNGFLDV
FTSFGGLVAEAFGFKSDPKKSDVKTYFTTVAAKLEKTKTDLNSLPKEKS
DISSTTGKPDSTGSVGTAVEGAIKEVSELLDKLVKAVKTAEGASSGTAA
IGEVVDNAAAAKAADKDSVTGIAKGIKEIVEAAGGSKKLKAAAAKGENN
KGAGKLFGKAGDAAHGDSEAASKAAGAVSAVSGEQILSAIVKAAAAGDQ
EGKKPGEAKNPIAAAIGEGDGDAEFNQDGMKKDDQIAAAIALRGMAKDG
KFAVKNDEKGKAEGAIKGAAESAVRKVLGAITGLIGDAVSSGLRKVGDS
VKAASKETPPALNK

In some embodiments, the amino acid sequence of OsPC is derived from Borrelia burgdorferi. A non-limiting example of the amino acid sequence of OspC is accessible through UniProtKB Accession No. Q07337, and is provided here as SEQ ID NO: 3:

MKKNTLSAILMTLFLFISCNNSGKDGNTSANSADESVKGPNLTEISKKI
TDSNAVLLAVKEVEALLSSIDEIAAKAIGKKIHQNNGLDTENNHNGSLL
AGAYAISTLIKQKLDGLKNEGLKEKIDAAKKCSETFTNKLKEKHTDLGK
EGVTDADAKEAILKTNGTKTKGAEELGKLFESVEVLSKAAKEMLANSVK
ELTSPVVAESPKKP

A non-limiting example of the amino acid sequence of BBK07 is accessible through GenBank Accession No. AAC66153.1, and is provided here as SEQ ID NO: 4:

MSKLILAISILLIISCKWHVDNPIDEATAESKSALTSVDQVLDEISEAT
GLSSEKITKLTPEELENLAKEAQDDSEKSKKEIEDQKNTKESKNIEVKD
TPRLIKLIKNSSEKIDSVFQTLINIGYNATYAAKSNLKNGLKMVKLLDE
LLKISVSSNGDKSTQKYNELKTVVNRFNAENSAIKVPLENGSKIEAKKC
IKTLMTNVETYFKGVSTELKDKKDDKYTKILAALSEAANKIENAAMAIH
LCFNN

A non-limiting example of the amino acid sequence of OppA2 is accessible through UniProtKB Accession No. Q6RH12, and is provided here as SEQ ID NO: 5:

RAGWIGDYADPLTFLSIFTQGYTQFSSHNYSNPEYNELIKKSDLELDPI
KRQDILRQAEEIIIEKDFPIAPIYIYGNSYLFRNDKWTGWNTNFLERFD
LCQLKLKNK

A non-limiting example of the amino acid sequence of Decorin Binding Protein A is accessible through UniProtKB Accession No. A0A0J9X1X0, and is provided here as SEQ ID NO: 6:

GLKGETKIILERSAKDITDEINKIKKDAADNNVNFAAFTDSETGSKVSE
NSFILEAKVRATTVAEKFVTAIEGEATKLKKTGSSGEFSAMYNMMLEVS
GPLEELGVLRMTKTVTDAAEQHPTTTAEGILEIAKIMKTKLQRVHTKNY
CALEKKKNPNFTDEKCKNN

A non-limiting example of the amino acid sequence of C6 is provided here as SEQ ID NO: 7:

MKKDDQIAAAIALRGMAKDGKFAVK

A non-limiting example of the amino acid sequence of C6 is provided here as SEQ ID NO: 8:

MKKDDQIAAAMVLRGMAKDGQFALKD

A non-limiting example of the amino acid sequence of C6 is provided here as SEQ ID NO: 9:

MKKRNDKIVAAIVLRGVAKDGKFAAA

A non-limiting example of the amino acid sequence of C6 is provided here as SEQ ID NO: 10:

MKKSDKIAAAIVLRGVAKDGKFAVA

A non-limiting example of the amino acid sequence of C6 is provided here as SEQ ID NO: 11:

MKKRNDNIAAAIVLRGVAKSGKFAVA

A non-limiting example of the amino acid sequence of C6 is provided here as SEQ ID NO: 12:

MKKDDQIAAAMVLRGMAKDGQFALK

A non-limiting example of the amino acid sequence of C6 is provided here as SEQ ID NO: 13:

MKKSDKIAAAIVLRGVAKSGKFAVA

A non-limiting example of the amino acid sequence of C6 is provided here as SEQ ID NO: 14:

MKKNDQIAAAIVLRGMAKDGEFALK

A non-limiting example of the amino acid sequence of C6 is provided here as SEQ ID NO: 15:

MKKRNDNIAAAIVLRGVAKDGQFALK

A non-limiting example of the amino acid sequence of C10 is provided here as SEQ ID NO: 16:

PVVAESPKKP

A non-limiting example of the amino acid sequence of a BBK07-related peptide is provided here as SEQ ID NO: 17:

CKWHVDNPIDEATA

A non-limiting example of the amino acid sequence of a BBK07-related peptide is provided here as SEQ ID NO: 18:

ITKLTPEELENLAK

A non-limiting example of the amino acid sequence of a BBK07-related peptide is provided here as SEQ ID NO: 19:

EKSKKEIEDQKNTK

A non-limiting example of the amino acid sequence of a OppA2-related peptide is provided here as SEQ ID NO: 20:

IFFLTFLCCNNKERK

A non-limiting example of the amino acid sequence of a OppA2-related peptide is provided here as SEQ ID NO: 21:

YGQNWTNPENMVTSGPFKLKERIPNEKIVFEKNNK

A non-limiting example of the amino acid sequence of a OppA2-related peptide is provided here as SEQ ID NO: 22:

SDYYSSAVNAIYFYS

A non-limiting example of the amino acid sequence of a OppA2-related peptide is provided here as SEQ ID NO: 23:

SDYYSSAVNAIYFYSENTHIKPLD

A non-limiting example of the amino acid sequence of a OppA2-related peptide is provided here as SEQ ID NO: 24:

IYFYSFNTHIKPLD

A non-limiting example of the amino acid sequence of a OppA2-related peptide is provided here as SEQ ID NO: 25:

IYFYSFNTHIKPLDNVKIRKALTLA

A non-limiting example of the amino acid sequence of a OppA2-related peptide is provided here as SEQ ID NO: 26:

LAEAGYPNGNGFPILKLKYN

A non-limiting example of the amino acid sequence of a OppA2-related peptide is provided here as SEQ ID NO: 27:

KKICEFIQNQWKKNLNIDVE

A non-limiting example of the amino acid sequence of a OppA2-related peptide is provided here as SEQ ID NO: 28:

APIYIYGNSYLFRND

A non-limiting example of the amino acid sequence of a DbpA-related peptide is provided here as SEQ ID NO: 29:

NKTFNNLLKLTILVNLLISCGLTGA

A non-limiting example of the amino acid sequence of a DbpA-related peptide is provided here as SEQ ID NO: 30:

TILVNLLISCGLTGA

A non-limiting example of the amino acid sequence of a DbpA-related peptide is provided here as SEQ ID NO: 31:

PSESRAGNFILEAKVRATTVAE

A non-limiting example of the amino acid sequence of a DbpB-related peptide is provided here as SEQ ID NO: 32:

LVACSIGLVERTNAALESSS

A non-limiting example of the amino acid sequence of a DbpB-related peptide is provided here as SEQ ID NO: 33:

KDLKNKILKIKKEATGKGVLFEAFTGLKTG

A non-limiting example of the amino acid sequence of IDRm is provided here as SEQ ID NO: 34:

LQARILAVERYLKDQQLLGIWGCSGKLICTTTAP

A non-limiting example of the amino acid sequence of IDRm is provided here as SEQ ID NO: 35:

LQARVLAVERYLKDQKFLGLWGCSGKIICTTAAP

A non-limiting example of the amino acid sequence of IDRm is provided here as SEQ ID NO: 36:

LQARILAIERYLQDQQLLGIWGCSGKHICTTT

A non-limiting example of the amino acid sequence of a SARS-CoV-2-related peptide is provided here as SEQ ID NO: 37:

SNNLDSKVGGNYNYLYRLFRKSNLKPFERDISTEIYQAGSTPCNG
VEGFNCYFPLQSYGFQPTNGVGYQ

A non-limiting example of the amino acid sequence of a SARS-CoV-2-related peptide is provided here as SEQ ID NO: 38:

LFRKSNLKPFERDISTEIYQAGST

A non-limiting example of the amino acid sequence of a SARS-CoV-2-related peptide is provided here as SEQ ID NO: 39:

NGLTGTGVLTESNKKFLPFQQFGRDIADTTDAVRDPQTLE

A non-limiting example of the amino acid sequence of a SARS-CoV-2-related peptide is provided here as SEQ ID NO: 40:

ITPCSFGGVSVITPGTNTSNQVAVLYQDVNCTEVPVAIHAD

A non-limiting example of the amino acid sequence of a SARS-CoV-2-related peptide is provided here as SEQ ID NO: 41:

LYQDVNCTEVPVAIHADQLTPTWRVYSTGSNVFQTRAGC

A non-limiting example of the amino acid sequence of a SARS-CoV-2-related peptide is provided here as SEQ ID NO: 42:

PVAIHADQLTPTWRVYSTGSN

A non-limiting example of the amino acid sequence of a SARS-CoV-2-related peptide is provided here as SEQ ID NO: 43:

ASYQTQTNSPRRARSVASQSIIAYTMS

A non-limiting example of the amino acid sequence of a SARS-CoV-2-related peptide is provided here as SEQ ID NO: 44:

KFQIYKTPPIKDGGFNFSQILPDPSKPS

A non-limiting example of the amino acid sequence of a SARS-CoV-2-related peptide is provided here as SEQ ID NO: 45:

LPDPSKPSKRSFIEDLLFNKVTLADAGFIKQYGDCLGDIA

A non-limiting example of the amino acid sequence of a SARS-CoV-2-related peptide is provided here as SEQ ID NO: 46:

AYKTFPPTEPKKDKKKKADETQALPQRQKKQQTVTLLPAA

A non-limiting example of the amino acid sequence of a SARS-CoV-2-related peptide is provided here as SEQ ID NO: 47:

QRQKKQQTVTLLPAADLDDFSKQLQQS

A non-limiting example of the amino acid sequence of a Dengue-related peptide is provided here as SEQ ID NO: 48:

MNNQRKKARN

A non-limiting example of the amino acid sequence of a Dengue-related peptide is provided here as SEQ ID NO: 49:

NVLRGFRKEI

A non-limiting example of the amino acid sequence of a Dengue-related peptide is provided here as SEQ ID NO: 50:

MLNILNRRRR

A non-limiting example of the amino acid sequence of a Dengue-related peptide is provided here as SEQ ID NO: 51:

NNQRKKARNTPFNMLKR

A non-limiting example of the amino acid sequence of a Dengue-related peptide is provided here as SEQ ID NO: 52:

NVLRGFRKEIGRMLNIL

A non-limiting example of the amino acid sequence of a Dengue-related peptide is provided here as SEQ ID NO: 53:

KEIGRMLNILNRRRRTA

A non-limiting example of the amino acid sequence of a Dengue-related peptide is provided here as SEQ ID NO: 54:

ELCEDTMTYKCPRITEA

A non-limiting example of the amino acid sequence of a Dengue-related peptide is provided here as SEQ ID NO: 55:

AEPDDVDCWCNATDTWV

A non-limiting example of the amino acid sequence of a Dengue-related peptide is provided here as SEQ ID NO: 56:

QRVETWALRHPGFTVIAL

A non-limiting example of the amino acid sequence of a Dengue-related peptide is provided here as SEQ ID NO: 57:

IVSRQEKGKS

A non-limiting example of the amino acid sequence of a Dengue-related peptide is provided here as SEQ ID NO: 58:

KQNEPEDI

A non-limiting example of the amino acid sequence of a Dengue-related peptide is provided here as SEQ ID NO: 59:

KGKSLLFKTEDGVNMC

A non-limiting example of the amino acid sequence of a Dengue-related peptide is provided here as SEQ ID NO: 60:

HITEVEPEDIDCWCNLT

A non-limiting example of the amino acid sequence of a Dengue-related peptide is provided here as SEQ ID NO: 61:

TSTWVTYGTCNQAG

A non-limiting example of the amino acid sequence of a Dengue-related peptide is provided here as SEQ ID NO: 62:

LFKTASGINMCTLIAMDL

A non-limiting example of the amino acid sequence of a Dengue-related peptide is provided here as SEQ ID NO: 63:

DRGWGNGCGLFG

A non-limiting example of the amino acid sequence of a Dengue-related peptide is provided here as SEQ ID NO: 64:

RGARRMAIL

A non-limiting example of the amino acid sequence of a Dengue-related peptide is provided here as SEQ ID NO: 65:

KGMSYSMCTGKFKIVKEI

A non-limiting example of the amino acid sequence of a Dengue-related peptide is provided here as SEQ ID NO: 66:

RVQYEGDGSPCKIPFEIM

A non-limiting example of the amino acid sequence of a Dengue-related peptide is provided here as SEQ ID NO: 67:

GSPCKIPFEIMDLEKRHV

A non-limiting example of the amino acid sequence of a Dengue-related peptide is provided here as SEQ ID NO: 68:

RCIGISNRDFVEGVSGGSWVDIVL

A non-limiting example of the amino acid sequence of a Dengue-related peptide is provided here as SEQ ID NO: 69:

NTTTASRCPTQGEP

A non-limiting example of the amino acid sequence of a Dengue-related peptide is provided here as SEQ ID NO: 70:

MENKAWLVHRQWFLDLPLPWLPGADT

A non-limiting example of the amino acid sequence of a Dengue-related peptide is provided here as SEQ ID NO: 71:

KEIAETQHGTIVIRVQYEGDG

A non-limiting example of the amino acid sequence of a Dengue-related peptide is provided here as SEQ ID NO: 72:

WFKKGSSIGQMFETTMRGA

A non-limiting example of the amino acid sequence of a Dengue-related peptide is provided here as SEQ ID NO: 73:

RHVLGRLITVNPIVT

A non-limiting example of the amino acid sequence of a Dengue-related peptide is provided here as SEQ ID NO: 74:

EPGQLKLNWFKKGSS

A non-limiting example of the amino acid sequence of a Dengue-related peptide is provided here as SEQ ID NO: 75:

DSRCPTQGEAVLPEE

A non-limiting example of the amino acid sequence of a Dengue-related peptide is provided here as SEQ ID NO: 76:

TQGEAVLPEEQDPNY

A non-limiting example of the amino acid sequence of a Dengue-related peptide is provided here as SEQ ID NO: 77:

DSGCVVSWKNKELKC

A non-limiting example of the amino acid sequence of a Dengue-related peptide is provided here as SEQ ID NO: 78:

LTLNLITEMG

A non-limiting example of the amino acid sequence of a Dengue-related peptide is provided here as SEQ ID NO: 79:

PTFMTQKARD

A non-limiting example of the amino acid sequence of a Dengue-related peptide is provided here as SEQ ID NO: 80:

ATREAQKR

A non-limiting example of the amino acid sequence of a Chagas-related peptide is provided here as SEQ ID NO: 81:

FGQAAAGDKPPP

A non-limiting example of the amino acid sequence of a Chagas-related peptide is provided here as SEQ ID NO: 82:

FGQAAAGDKPAP

A non-limiting example of the amino acid sequence of a Chagas-related peptide is provided here as SEQ ID NO: 83:

FGQAAAGDKPSL

In some embodiments, sample is a biological sample. In some embodiments, the biological sample is selected from the group consisting of: saliva, oral fluid, tears, urine, interstitial fluid, synovial fluid, and cerebrospinal fluid. In some embodiments, the biological sample is blood. In some embodiments, the biological sample is blood, serum, plasma, or a blood fraction. In some embodiments, the blood is collected from a subject suspected of having a Lyme disease infection. In some embodiments, the blood is collected from a subject who is not suspected of having a Lyme disease infection. In some embodiments, a subject who is not suspected of having a Lyme disease infection displays symptoms consistent with a Lyme disease infection. In some embodiments, a subject who is not suspected of having a Lyme disease infection traveled to an area that is associated with Lyme disease infection.

In some embodiments, the subject is a human or a non-human animal. In some embodiments, the subject is a rodent, a non-human primate, a canine, a feline, or another mammal. In some embodiments, the subject is a human. In some embodiments, the sample is collected from a subject suspected of having a disease caused by a pathogenic member of the bacterial genus Borrelia. In some embodiments, the sample is collected from a subject who is not suspected of having a disease caused by a pathogenic member of the bacterial genus Borrelia. In some embodiments, the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica. In some embodiments, the pathogenic member of the bacterial genus Borrelia is Borrelia burgdorferi.

In some embodiments, the first antigen is a peptide or protein comprising an amino acid sequence having at least 70% sequence identity to the amino acid sequence set forth in any one of SEQ ID NOs: 1-83. In some embodiments, the first antigen is a protein comprising an amino acid sequence having at least 70% sequence identity to the amino acid sequence set forth in any one of SEQ ID NO: 1-6. In some embodiments, the second antigen is a peptide or protein comprising an amino acid sequence having at least 70% sequence identity to the amino acid sequence set forth in any one of SEQ ID NOs: 1-83. In some embodiments, the second antigen is a peptide comprising an amino acid sequence having at least 70% sequence identity to the amino acid sequence set forth in any one of SEQ ID NOS: 7-83. In some embodiments, the sequence identity is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 100%.

In some embodiments, the method further comprises a wash step in between the steps of contacting the antigens with the antibody and assessing the presence of a signal from the detectable label. In some embodiments, the wash step comprises adding a wash buffer to the vessel and incubating the wash buffer in the vessel for a sufficient time to remove unbound antibodies.

In some embodiments, the detectable label is a luminescent label, an enzymatic or colorimetric label, or a radiometric label. In some embodiments, the luminescent label is a luminescent dye, a FRET label, a protein label, or a fluorescent protein. In some embodiments, the luminescent label is a lanthanide, a fluorescent label, or an organic dye. In some embodiments, the lanthanide is europium, terbium, or any other fluorescent lanthanide. In some embodiments, the protein label is phycoerythrin or other phycobiliproteins. In some embodiments, the fluorescent or luminescent label is a dye. In some embodiments, the fluorescent or luminescent label is fluorescein isothiocyanate, rhodamine, Alexa Fluor® series, Spark™ series (BioLegend), Brilliant Violet™ (BioLegend), Cy3, Cy5, Quantum dots, or Polymer dots. In some embodiments, the enzymatic label is a horseradish peroxidase or alkaline phosphatase label. In some embodiments, the detectable label requires an enzyme. In some embodiments, the enzyme is horseradish peroxidase, alkaline phosphatase, luciferase, beta glucosidase, beta galactosidase, or glucose oxidase. In some embodiments, the colorimetric label is colloidal gold, colloidal carbon, a latex particle dyed with a visible dye, or any particle comprising a visible dye.

In some embodiments, the method further comprises a step of detecting the detectable label between the steps of contacting the antigens with the antibody and assessing the presence of a signal produced by said detectable label. In some embodiments, the method further comprises a step of detecting the detectable label after contacting the first antigen with the antibody and before contacting the second antigen with the antibody. In some embodiments, the method further comprises a step of detecting the detectable label after contacting the second antigen with the antibody and before contacting the first antigen with the antibody. In some embodiments, the step of assessing the presence of a signal comprises detecting signal pulses from the microplate wells.

In some embodiments, the detectable label is adapted to be detected by detecting a signal pulse emitted by the detectable label. In some embodiments, the step of detecting comprises adding a substrate to the microplate wells. In some embodiments, the detectable label is adapted to be detected by a substrate. In some embodiments, the substrate is selected from the group consisting of: ABTS, OPD, AmplexRed, DAB, AEC, TMB, Homovanillic Acid, or Luminol. In some embodiments, the substrate is 3,3′,5,5′-tetramethylbenzidine (TMB). In some embodiments, the substrate is Luminol.

In some embodiments, the vessel is adapted to receive at least a portion of the sample. In some embodiments, the vessel is a well. In some embodiments, the vessel is a well of a microplate comprising one or more wells. In some embodiments, the presence of a signal is also indicative of the presence of the antibody in the sample. In some embodiments, the presence of a signal is suggestive of a Lyme disease infection in the subject. In some embodiments, the presence of a signal is indicative of Lyme disease-specific antibodies. In some embodiments, the presence of a signal is indicative of a previous or active Lyme disease infection in the subject. In some embodiments, the presence of a signal is indicative of an active Lyme disease infection in the subject.

In some embodiments, the method further comprises administering an antibiotic to the subject from whom the sample was obtained. In some embodiments, the antibiotic is selected from the group consisting of: amoxicillin, cefuroxime, doxycycline, and ceftriaxone. In some embodiments, the antibiotic is doxycycline.

In some embodiments, the first antigen-binding site and the second antigen-binding site are identical. In some embodiments, the first antigen-binding site and the second antigen-binding site share at least 90% sequence identity. In some embodiments, the first antigen-binding site and the second antigen-binding site share at least 91%, at least 92%, at least 93%, at least 94%, at least 95%, at least 96%, at least 97%, at least 98%, at least 99%, at least 99.5%, or 100% sequence identity. In some embodiments, the first antigen is capable of binding to the first antigen-binding site or the second antigen-binding site. In some embodiments, the second antigen is capable of binding to the first antigen-binding site or the second antigen-binding site.

Aspects of the present disclosure relate to a method of detecting the presence of an antibody in a sample, the method comprising: adding the sample to a vessel comprising a surface coated with a first binding molecule; adding a first antigen conjugated to a second binding molecule to the vessel and incubating the sample with the first antigen; adding a second antigen conjugated to a detectable label to the vessel and incubating the sample with the second antigen; and assessing the presence of a signal from the detectable label; wherein: the presence of a signal is indicative of association of the antibody with the first antigen and the second antigen; incubating the first antigen and the second antigen with the sample is sufficient to bind the first antigen to a first antigen-binding site on the antibody and the second antigen to a second antigen-binding site on the antibody, if present; the first binding molecule binds to the second binding molecule; and the first antigen and the second antigen are different. In some embodiments, the first binding molecule is a protein. In some embodiments, the first binding molecule is a peptide. In some embodiments, the second binding molecule is a protein. In some embodiments, the second binding molecule is a peptide.

In some embodiments, each of the steps described herein are conducted in order. In some embodiments, each of the steps described herein are not conducted in order. In some embodiments, each of the steps described herein are conducted simultaneously. In some embodiments, a portion of the steps described herein are conducted simultaneously. In some embodiments, the steps of contacting the first and second antigens with an antibody are conducted consecutively. In some embodiments, the steps of contacting the first and second antigens with an antibody are conducted simultaneously. In some embodiments, the first antigen is a recombinant protein. In some embodiments, the second antigen is a peptide derived from the recombinant protein. In some embodiments, the second antigen is a recombinant protein. In some embodiments, the first antigen is a peptide derived from the recombinant protein. In some embodiments, the first antigen is a first peptide and the second antigen is a second peptide.

In some embodiments, the first antigen is selected from a group consisting of: VlsE, C6, OspC, C10, BBK07, OppA2, and Decorin Binding Protein A. In some embodiments, the second antigen is selected from a group consisting of: VlsE, C6, OspC, C10, BBK07, OppA2, and Decorin Binding Protein A. In some embodiments, the first antigen is VlsE and the second antigen is C6. In some embodiments, the first antigen is C6 and the second antigen is VlsE. In some embodiments, the first antigen is OspC and the second antigen is C10. In some embodiments, the first antigen is C10 and the second antigen is OspC.

In some embodiments, the first binding molecule is an avidin protein and the second binding molecule is a biotin. In some embodiments, the avidin protein is NeutrAvidin or streptavidin. In some embodiments, the avidin protein is streptavidin.

In some embodiments, sample is a biological sample. In some embodiments, the biological sample is selected from the group consisting of: saliva, oral fluid, tears, urine, interstitial fluid, synovial fluid, and cerebrospinal fluid. In some embodiments, the biological sample is blood. In some embodiments, the biological sample is blood, serum, plasma, or a blood fraction. In some embodiments, the blood is collected from a subject suspected of having a Lyme disease infection. In some embodiments, the blood is collected from a subject who is not suspected of having a Lyme disease infection.

In some embodiments, the subject is a human or a non-human animal. In some embodiments, the subject is a human. In some embodiments, the sample is collected from a subject suspected of having a disease caused by a pathogenic member of the bacterial genus Borrelia. In some embodiments, the sample is collected from a subject who is not suspected of having a disease caused by a pathogenic member of the bacterial genus Borrelia. In some embodiments, the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica. In some embodiments, the pathogenic member of the bacterial genus Borrelia is Borrelia burgdorferi.

In some embodiments, the first antigen is a peptide or protein comprising an amino acid sequence having at least 70% sequence identity to the amino acid sequence set forth in any one of SEQ ID NOs: 1-83. In some embodiments, the first antigen is a protein comprising an amino acid sequence having at least 70% sequence identity to the amino acid sequence set forth in any one of SEQ ID NO: 1-6. In some embodiments, the second antigen is a peptide or protein comprising an amino acid sequence having at least 70% sequence identity to the amino acid sequence set forth in any one of SEQ ID NOs: 1-83. In some embodiments, the second antigen is a peptide comprising an amino acid sequence having at least 70% sequence identity to the amino acid sequence set forth in any one of SEQ ID NOS: 7-83. In some embodiments, the sequence identity is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 100%.

In some embodiments, the method further comprises a wash step in between the steps of contacting the antigens with the antibody and assessing the presence of a signal from the detectable label. In some embodiments, the wash step comprises adding a wash buffer to the vessel and incubating the wash buffer in the vessel for a sufficient time to remove unbound antibodies.

In some embodiments, the detectable label is a luminescent label, an enzymatic or colorimetric label, or a radiometric label. In some embodiments, the luminescent label is a luminescent dye, a FRET label, or a fluorescent protein. In some embodiments, the luminescent label is a lanthanide, a fluorescent label, or an organic dye. In some embodiments, the colorimetric label is a horseradish peroxidase label or alkaline phosphatase label. In some embodiments, the colorimetric label is colloidal gold, colloidal carbon, a latex particle dyed with a visible dye, or any particle comprising a visible dye.

In some embodiments, the method further comprises a step of detecting the detectable label between the steps of contacting the antigens with the antibody and assessing the presence of a signal produced by said detectable label. In some embodiments, the method further comprises a step of detecting the detectable label after contacting the first antigen with the antibody and before contacting the second antigen with the antibody. In some embodiments, the method further comprises a step of detecting the detectable label after contacting the second antigen with the antibody and before contacting the first antigen with the antibody.

In some embodiments, the step of assessing the presence of a signal comprises detecting signal pulses from the microplate wells. In some embodiments, the detectable label is adapted to be detected by detecting a signal pulse emitted by the detectable label. In some embodiments, the step of detecting comprises adding a substrate to the microplate wells. In some embodiments, the detectable label is adapted to be detected by a substrate.

In some embodiments, the substrate is selected from the group consisting of: ABTS, OPD, AmplexRed, DAB, AEC, TMB, Homovanillic Acid, or Luminol. In some embodiments, the substrate is 3,3′,5,5′-tetramethylbenzidine (TMB). In some embodiments, the substrate is Luminol.

In some embodiments, the vessel is adapted to receive at least a portion of the sample. In some embodiments, the vessel is a well. In some embodiments, the vessel is a well of a microplate comprising one or more wells.

In some embodiments, the presence of a signal is also indicative of the presence of the antibody in the sample. In some embodiments, the presence of a signal is suggestive of a Lyme disease infection in the subject. In some embodiments, the presence of a signal is indicative of Lyme disease-specific antibodies. In some embodiments, the presence of a signal is indicative of a previous or active Lyme disease infection in the subject. In some embodiments, the presence of a signal is indicative of an active Lyme disease infection in the subject.

In some embodiments, the method further comprises administering an antibiotic to the subject from whom the sample was obtained. In some embodiments, the antibiotic is selected from the group consisting of: amoxicillin, cefuroxime, doxycycline, and ceftriaxone. In some embodiments, the antibiotic is doxycycline.

In some embodiments, the first antigen-binding site and the second antigen-binding site are identical. In some embodiments, the first antigen-binding site and the second antigen-binding site share at least 90% sequence identity. In some embodiments, the first antigen-binding site and the second antigen-binding site share at least 91%, at least 92%, at least 93%, at least 94%, at least 95%, at least 96%, at least 97%, at least 98%, at least 99%, at least 99.5%, or 100% sequence identity. In some embodiments, the first antigen is capable of binding to the first antigen-binding site or the second antigen-binding site. In some embodiments, the second antigen is capable of binding to the first antigen-binding site or the second antigen-binding site.

Aspects of the present disclosure relate to a system comprising: a microplate comprising a plurality of wells; and a first antigen immobilized or adapted to be immobilized to at least one of the plurality of wells; wherein: at least one of the plurality of wells including the first antigen is adapted to receive: a sample to be analyzed for the presence of an antibody; and a second antigen conjugated to a detectable label; wherein: the first antigen binds to a first antigen-binding site on the antibody and the second antigen binds to a second antigen-binding site on the antibody, if present; and the first antigen and the second antigen are different.

In some embodiments, the first antigen is a recombinant protein. In some embodiments, the second antigen is a peptide derived from the recombinant protein. In some embodiments, the second antigen is a recombinant protein. In some embodiments, the first antigen is a peptide derived from the recombinant protein. In some embodiments, the first antigen is a first peptide and the second antigen is a second peptide.

In some embodiments, the first antigen is selected from a group consisting of: VlsE, C6, OspC, C10, BBK07, OppA2, and Decorin Binding Protein A. In some embodiments, the second antigen is selected from a group consisting of: VlsE, C6, OspC, C10, BBK07, OppA2, and Decorin Binding Protein A. In some embodiments, the first antigen is VlsE and the second antigen is C6. In some embodiments, the first antigen is C6 and the second antigen is VlsE. In some embodiments, the first antigen is OspC and the second antigen is C10. In some embodiments, the first antigen is C10 and the second antigen is OspC.

In some embodiments, sample is a biological sample. In some embodiments, the biological sample is selected from the group consisting of: saliva, oral fluid, tears, urine, interstitial fluid, synovial fluid, and cerebrospinal fluid. In some embodiments, the biological sample is blood. In some embodiments, the biological sample is blood, serum, plasma, or a blood fraction. In some embodiments, the blood is collected from a subject suspected of having a Lyme disease infection. In some embodiments, the blood is collected from a subject who is not suspected of having a Lyme disease infection. In some embodiments, the subject is a human or a non-human animal.

In some embodiments, the subject is a human. In some embodiments, the sample is collected from a subject suspected of having a disease caused by a pathogenic member of the bacterial genus Borrelia. In some embodiments, the sample is collected from a subject who is not suspected of having a disease caused by a pathogenic member of the bacterial genus Borrelia. In some embodiments, the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica. In some embodiments, the pathogenic member of the bacterial genus Borrelia is Borrelia burgdorferi.

In some embodiments, the first antigen is a peptide or protein comprising an amino acid sequence having at least 70% sequence identity to the amino acid sequence set forth in any one of SEQ ID NOs: 1-83. In some embodiments, the first antigen is a protein comprising an amino acid sequence having at least 70% sequence identity to the amino acid sequence set forth in any one of SEQ ID NO: 1-6. In some embodiments, the second antigen is a peptide or protein comprising an amino acid sequence having at least 70% sequence identity to the amino acid sequence set forth in any one of SEQ ID NOs: 1-83. In some embodiments, the second antigen is a peptide comprising an amino acid sequence having at least 70% sequence identity to the amino acid sequence set forth in any one of SEQ ID NOS: 7-83. In some embodiments, the sequence identity is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 100%.

In some embodiments, the detectable label is a luminescent label, an enzymatic or colorimetric label, or a radiometric label. In some embodiments, the luminescent label is a luminescent dye, a FRET label, or a fluorescent protein. In some embodiments, the luminescent label is a lanthanide, a fluorescent label, or an organic dye. In some embodiments, the colorimetric label is a horseradish peroxidase label or alkaline phosphatase label. In some embodiments, the colorimetric label is colloidal gold, colloidal carbon, a latex particle dyed with a visible dye, or any particle comprising a visible dye.

In some embodiments, the detectable label is adapted to be detected by detecting a signal pulse emitted by the detectable label. In some embodiments, the step of detecting comprises adding a substrate to the microplate wells. In some embodiments, the detectable label is adapted to be detected by a substrate. In some embodiments, the substrate is selected from the group consisting of: ABTS, OPD, AmplexRed, DAB, AEC, TMB, Homovanillic Acid, or Luminol. In some embodiments, the substrate is 3,3′,5,5′-tetramethylbenzidine (TMB). In some embodiments, the substrate is Luminol.

In some embodiments, the vessel is adapted to receive at least a portion of the sample. In some embodiments, the vessel is a well. In some embodiments, the vessel is a well of a microplate comprising one or more wells.

In some embodiments, the presence of a signal is also indicative of the presence of the antibody in the sample. In some embodiments, the presence of a signal is suggestive of a Lyme disease infection in the subject. In some embodiments, the presence of a signal is indicative of Lyme disease-specific antibodies. In some embodiments, the presence of a signal is indicative of a previous or active Lyme disease infection in the subject. In some embodiments, the presence of a signal is indicative of an active Lyme disease infection in the subject.

In some embodiments, the first antigen-binding site and the second antigen-binding site are identical. In some embodiments, the first antigen-binding site and the second antigen-binding site share at least 90% sequence identity. In some embodiments, the first antigen-binding site and the second antigen-binding site share at least 91%, at least 92%, at least 93%, at least 94%, at least 95%, at least 96%, at least 97%, at least 98%, at least 99%, at least 99.5%, or 100% sequence identity. In some embodiments, the first antigen is capable of binding to the first antigen-binding site or the second antigen-binding site. In some embodiments, the second antigen is capable of binding to the first antigen-binding site or the second antigen-binding site.

In some embodiments, the first antigen is capable of binding to the first antigen-binding site or the second antigen-binding site. In some embodiments, the second antigen is capable of binding to the first antigen-binding site or the second antigen-binding site.

Aspects of the present disclosure relate to a microplate, comprising: a plurality of wells; and a first antigen immobilized to a surface of at least one of the plurality of wells; wherein: the microplate is adapted to receive: a sample to be analyzed for the presence of an antibody; and a second antigen conjugated to a detectable label in at least one of the plurality of wells including the first antigen; the first antigen binds to a first antigen-binding site on the antibody and the second antigen binds to a second antigen-binding site on the antibody, if present; and the first antigen and the second antigen are different.

In some embodiments, the first antigen is a recombinant protein. In some embodiments, the second antigen is a peptide derived from the recombinant protein. In some embodiments, the second antigen is a recombinant protein. In some embodiments, the first antigen is a peptide derived from the recombinant protein. In some embodiments, the first antigen is a first peptide and the second antigen is a second peptide.

In some embodiments, the first antigen is selected from a group consisting of: VlsE, C6, OspC, C10, BBK07, OppA2, and Decorin Binding Protein A. In some embodiments, the second antigen is selected from a group consisting of: VlsE, C6, OspC, C10, BBK07, OppA2, and Decorin Binding Protein A. In some embodiments, the first antigen is VlsE and the second antigen is C6. In some embodiments, the first antigen is C6 and the second antigen is VlsE. In some embodiments, the first antigen is OspC and the second antigen is C10. In some embodiments, the first antigen is C10 and the second antigen is OspC.

In some embodiments, sample is a biological sample. In some embodiments, the biological sample is selected from the group consisting of: saliva, oral fluid, tears, urine, interstitial fluid, synovial fluid, and cerebrospinal fluid. In some embodiments, the biological sample is blood. In some embodiments, the biological sample is blood, serum, plasma, or a blood fraction. In some embodiments, the blood is collected from a subject suspected of having a Lyme disease infection. In some embodiments, the blood is collected from a subject who is not suspected of having a Lyme disease infection.

In some embodiments, the subject is a human or a non-human animal. In some embodiments, the subject is a human. In some embodiments, the sample is collected from a subject suspected of having a disease caused by a pathogenic member of the bacterial genus Borrelia. In some embodiments, the sample is collected from a subject who is not suspected of having a disease caused by a pathogenic member of the bacterial genus Borrelia. In some embodiments, the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica. In some embodiments, the pathogenic member of the bacterial genus Borrelia is Borrelia burgdorferi.

In some embodiments, the first antigen is a peptide or protein comprising an amino acid sequence having at least 70% sequence identity to the amino acid sequence set forth in any one of SEQ ID NOs: 1-83. In some embodiments, the first antigen is a protein comprising an amino acid sequence having at least 70% sequence identity to the amino acid sequence set forth in any one of SEQ ID NO: 1-6. In some embodiments, the second antigen is a peptide or protein comprising an amino acid sequence having at least 70% sequence identity to the amino acid sequence set forth in any one of SEQ ID NOs: 1-83. In some embodiments, the second antigen is a peptide comprising an amino acid sequence having at least 70% sequence identity to the amino acid sequence set forth in any one of SEQ ID NOs: 7-83. In some embodiments, the sequence identity is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 100%.

In some embodiments, the detectable label is a luminescent label, an enzymatic or colorimetric label, or a radiometric label. In some embodiments, the luminescent label is a luminescent dye, a FRET label, or a fluorescent protein. In some embodiments, the luminescent label is a lanthanide, a fluorescent label, or an organic dye. In some embodiments, the colorimetric label is a horseradish peroxidase label or alkaline phosphatase label. In some embodiments, the colorimetric label is colloidal gold, colloidal carbon, a latex particle dyed with a visible dye, or any particle comprising a visible dye.

In some embodiments, the detectable label is adapted to be detected by detecting a signal pulse emitted by the detectable label. In some embodiments, the detectable label is adapted to be detected by a substrate. In some embodiments, the substrate is selected from the group consisting of: ABTS, OPD, AmplexRed, DAB, AEC, TMB, Homovanillic Acid, or Luminol. In some embodiments, the substrate is 3,3′,5,5′-tetramethylbenzidine (TMB). In some embodiments, the substrate is Luminol.

In some embodiments, the vessel is adapted to receive at least a portion of the sample. In some embodiments, the vessel is a well. In some embodiments, the vessel is a well of a microplate comprising one or more wells.

In some embodiments, the presence of a signal is also indicative of the presence of the antibody in the sample. In some embodiments, the presence of a signal is suggestive of a Lyme disease infection in the subject. In some embodiments, the presence of a signal is indicative of Lyme disease-specific antibodies. In some embodiments, the presence of a signal is indicative of a previous or active Lyme disease infection in the subject. In some embodiments, the presence of a signal is indicative of an active Lyme disease infection in the subject.

In some embodiments, the first antigen-binding site and the second antigen-binding site are identical. In some embodiments, the first antigen-binding site and the second antigen-binding site share at least 90% sequence identity. In some embodiments, the first antigen-binding site and the second antigen-binding site share at least 91%, at least 92%, at least 93%, at least 94%, at least 95%, at least 96%, at least 97%, at least 98%, at least 99%, at least 99.5%, or 100% sequence identity. In some embodiments, the first antigen is capable of binding to the first antigen-binding site or the second antigen-binding site. In some embodiments, the second antigen is capable of binding to the first antigen-binding site or the second antigen-binding site. Aspects of the present disclosure relate to a microplate, comprising: a plurality of wells; a first antigen immobilized to a surface of at least one of the plurality of wells; a second antigen disposed in at least one of the plurality of wells including the first antigen; and an antibody bound to the first antigen and the second antigen; wherein: the second antigen is conjugated to a detectable label; and the first antigen and the second antigen are different.

In some embodiments, the first antigen is a recombinant protein. In some embodiments, the second antigen is a peptide derived from the recombinant protein. In some embodiments, the second antigen is a recombinant protein. In some embodiments, the first antigen is a peptide derived from the recombinant protein. In some embodiments, the first antigen is a first peptide and the second antigen is a second peptide.

In some embodiments, the first antigen is selected from a group consisting of: VlsE, C6, OspC, C10, BBK07, OppA2, and Decorin Binding Protein A. In some embodiments, the second antigen is selected from a group consisting of: VlsE, C6, OspC, C10, BBK07, OppA2, and Decorin Binding Protein A. In some embodiments, the first antigen is VlsE and the second antigen is C6. In some embodiments, the first antigen is C6 and the second antigen is VlsE. In some embodiments, the first antigen is OspC and the second antigen is C10. In some embodiments, the first antigen is C10 and the second antigen is OspC.

In some embodiments, sample is a biological sample. In some embodiments, the biological sample is selected from the group consisting of: saliva, oral fluid, tears, urine, interstitial fluid, synovial fluid, and cerebrospinal fluid. In some embodiments, the biological sample is blood. In some embodiments, the biological sample is blood, serum, plasma, or a blood fraction. In some embodiments, the blood is collected from a subject suspected of having a Lyme disease infection. In some embodiments, the blood is collected from a subject who is not suspected of having a Lyme disease infection.

In some embodiments, the subject is a human or a non-human animal. In some embodiments, the subject is a human. In some embodiments, the sample is collected from a subject suspected of having a disease caused by a pathogenic member of the bacterial genus Borrelia. In some embodiments, the sample is collected from a subject who is not suspected of having a disease caused by a pathogenic member of the bacterial genus Borrelia. In some embodiments, the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica. In some embodiments, the pathogenic member of the bacterial genus Borrelia is Borrelia burgdorferi.

In some embodiments, the first antigen is a peptide or protein comprising an amino acid sequence having at least 70% sequence identity to the amino acid sequence set forth in any one of SEQ ID NOs: 1-83. In some embodiments, the first antigen is a protein comprising an amino acid sequence having at least 70% sequence identity to the amino acid sequence set forth in any one of SEQ ID NO: 1-6. In some embodiments, the second antigen is a peptide or protein comprising an amino acid sequence having at least 70% sequence identity to the amino acid sequence set forth in any one of SEQ ID NOs: 1-83. In some embodiments, the second antigen is a peptide comprising an amino acid sequence having at least 70% sequence identity to the amino acid sequence set forth in any one of SEQ ID NOS: 7-83. In some embodiments, the sequence identity is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 100%.

In some embodiments, the detectable label is a luminescent label, an enzymatic or colorimetric label, or a radiometric label. In some embodiments, the luminescent label is a luminescent dye, a FRET label, or a fluorescent protein. In some embodiments, the luminescent label is a lanthanide, a fluorescent label, or an organic dye. In some embodiments, the colorimetric label is a horseradish peroxidase label or alkaline phosphatase label. In some embodiments, the colorimetric label is colloidal gold, colloidal carbon, a latex particle dyed with a visible dye, or any particle comprising a visible dye. In some embodiments, the detectable label is adapted to be detected by detecting a signal pulse emitted by the detectable label. In some embodiments, the detectable label is adapted to be detected by a substrate. In some embodiments, the substrate is selected from the group consisting of: ABTS, OPD, AmplexRed, DAB, AEC, TMB, Homovanillic Acid, or Luminol. In some embodiments, the substrate is 3,3′,5,5′-tetramethylbenzidine (TMB). In some embodiments, the substrate is Luminol.

In some embodiments, the vessel is adapted to receive at least a portion of the sample. In some embodiments, the vessel is a well. In some embodiments, the vessel is a well of a microplate comprising one or more wells.

In some embodiments, the presence of a signal is also indicative of the presence of the antibody in the sample. In some embodiments, the presence of a signal is suggestive of a Lyme disease infection in the subject. In some embodiments, the presence of a signal is indicative of Lyme disease-specific antibodies. In some embodiments, the presence of a signal is indicative of a previous or active Lyme disease infection in the subject. In some embodiments, the presence of a signal is indicative of an active Lyme disease infection in the subject.

In some embodiments, the first antigen-binding site and the second antigen-binding site are identical. In some embodiments, the first antigen-binding site and the second antigen-binding site share at least 90% sequence identity. In some embodiments, the first antigen-binding site and the second antigen-binding site share at least 91%, at least 92%, at least 93%, at least 94%, at least 95%, at least 96%, at least 97%, at least 98%, at least 99%, at least 99.5%, or 100% sequence identity. In some embodiments, the first antigen is capable of binding to the first antigen-binding site or the second antigen-binding site. In some embodiments, the second antigen is capable of binding to the first antigen-binding site or the second antigen-binding site.

Aspects of the present disclosure relate to a kit, comprising: a microplate comprising a plurality of wells and a first antigen immobilized to a surface of at least one of the plurality of wells; and a reagent comprising a second antigen conjugated to a detectable label; wherein: the microplate is adapted to receive a sample to be analyzed for the presence of an antibody; and the first antigen and the second antigen are different.

In some embodiments, the first antigen is a recombinant protein. In some embodiments, the second antigen is a peptide derived from the recombinant protein. In some embodiments, the second antigen is a recombinant protein. In some embodiments, the first antigen is a peptide derived from the recombinant protein. In some embodiments, the first antigen is a first peptide and the second antigen is a second peptide.

In some embodiments, the first antigen is selected from a group consisting of: VlsE, C6, OspC, C10, BBK07, OppA2, and Decorin Binding Protein A. In some embodiments, the second antigen is selected from a group consisting of: VlsE, C6, OspC, C10, BBK07, OppA2, and Decorin Binding Protein A. In some embodiments, the first antigen is VlsE and the second antigen is C6. In some embodiments, the first antigen is C6 and the second antigen is VlsE. In some embodiments, the first antigen is OspC and the second antigen is C10. In some embodiments, the first antigen is C10 and the second antigen is OspC.

In some embodiments, sample is a biological sample. In some embodiments, the biological sample is selected from the group consisting of: saliva, oral fluid, tears, urine, interstitial fluid, synovial fluid, and cerebrospinal fluid. In some embodiments, the biological sample is blood. In some embodiments, the biological sample is blood, serum, plasma, or a blood fraction. In some embodiments, the blood is collected from a subject suspected of having a Lyme disease infection. In some embodiments, the blood is collected from a subject who is not suspected of having a Lyme disease infection.

In some embodiments, the subject is a human or a non-human animal. In some embodiments, the subject is a human. In some embodiments, the sample is collected from a subject suspected of having a disease caused by a pathogenic member of the bacterial genus Borrelia. In some embodiments, the sample is collected from a subject who is not suspected of having a disease caused by a pathogenic member of the bacterial genus Borrelia. In some embodiments, the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica. In some embodiments, the pathogenic member of the bacterial genus Borrelia is Borrelia burgdorferi.

In some embodiments, the first antigen is a peptide or protein comprising an amino acid sequence having at least 70% sequence identity to the amino acid sequence set forth in any one of SEQ ID NOs: 1-83. In some embodiments, the first antigen is a protein comprising an amino acid sequence having at least 70% sequence identity to the amino acid sequence set forth in any one of SEQ ID NO: 1-6. In some embodiments, the second antigen is a peptide or protein comprising an amino acid sequence having at least 70% sequence identity to the amino acid sequence set forth in any one of SEQ ID NOs: 1-83. In some embodiments, the second antigen is a peptide comprising an amino acid sequence having at least 70% sequence identity to the amino acid sequence set forth in any one of SEQ ID NOS: 7-83. In some embodiments, the sequence identity is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 100%.

In some embodiments, the detectable label is a luminescent label, an enzymatic or colorimetric label, or a radiometric label. In some embodiments, the luminescent label is a luminescent dye, a FRET label, or a fluorescent protein. In some embodiments, the luminescent label is a lanthanide, a fluorescent label, or an organic dye. In some embodiments, the colorimetric label is a horseradish peroxidase label or alkaline phosphatase label. In some embodiments, the colorimetric label is colloidal gold, colloidal carbon, a latex particle dyed with a visible dye, or any particle comprising a visible dye. In some embodiments, the detectable label is adapted to be detected by detecting a signal pulse emitted by the detectable label. In some embodiments, the detectable label is adapted to be detected by a substrate. In some embodiments, the substrate is selected from the group consisting of: ABTS, OPD, AmplexRed, DAB, AEC, TMB, Homovanillic Acid, or Luminol. In some embodiments, the substrate is 3,3′,5,5′-tetramethylbenzidine (TMB). In some embodiments, the substrate is Luminol.

In some embodiments, the vessel is adapted to receive at least a portion of the sample. In some embodiments, the vessel is a well. In some embodiments, the vessel is a well of a microplate comprising one or more wells.

In some embodiments, the presence of a signal is also indicative of the presence of the antibody in the sample. In some embodiments, the presence of a signal is suggestive of a Lyme disease infection in the subject. In some embodiments, the presence of a signal is indicative of Lyme disease-specific antibodies. In some embodiments, the presence of a signal is indicative of a previous or active Lyme disease infection in the subject. In some embodiments, the presence of a signal is indicative of an active Lyme disease infection in the subject.

In some embodiments, the first antigen-binding site and the second antigen-binding site are identical. In some embodiments, the first antigen-binding site and the second antigen-binding site share at least 90% sequence identity. In some embodiments, the first antigen-binding site and the second antigen-binding site share at least 91%, at least 92%, at least 93%, at least 94%, at least 95%, at least 96%, at least 97%, at least 98%, at least 99%, at least 99.5%, or 100% sequence identity. In some embodiments, the first antigen is capable of binding to the first antigen-binding site or the second antigen-binding site. In some embodiments, the second antigen is capable of binding to the first antigen-binding site or the second antigen-binding site.

Aspects of the present disclosure relate to a kit, comprising: a microplate comprising a plurality of wells, wherein the plurality of wells are coated with a first binding molecule; a reagent comprising a first antigen conjugated to a second binding molecule; and a reagent comprising a second antigen conjugated to a detectable label; wherein: the microplate is adapted to receive a sample to be analyzed for the presence of an antibody; and the first antigen and the second antigen are different. In some embodiments, the first binding molecule is a protein. In some embodiments, the first binding molecule is a peptide. In some embodiments, the second binding molecule is a protein. In some embodiments, the second binding molecule is a peptide.

In some embodiments, the first antigen is a recombinant protein. In some embodiments, the second antigen is a peptide derived from the recombinant protein. In some embodiments, the second antigen is a recombinant protein. In some embodiments, the first antigen is a peptide derived from the recombinant protein. In some embodiments, the first antigen is a first peptide and the second antigen is a second peptide.

In some embodiments, the first antigen is selected from a group consisting of: VlsE, C6, OspC, C10, BBK07, OppA2, and Decorin Binding Protein A. In some embodiments, the second antigen is selected from a group consisting of: VlsE, C6, OspC, C10, BBK07, OppA2, and Decorin Binding Protein A. In some embodiments, the first antigen is VlsE and the second antigen is C6. In some embodiments, the first antigen is C6 and the second antigen is VlsE. In some embodiments, the first antigen is OspC and the second antigen is C10. In some embodiments, the first antigen is C10 and the second antigen is OspC.

In some embodiments, the first binding molecule is an avidin protein and the second binding molecule is a biotin. In some embodiments, the avidin protein is NeutrAvidin or streptavidin. In some embodiments, the avidin protein is streptavidin.

In some embodiments, sample is a biological sample. In some embodiments, the biological sample is selected from the group consisting of: saliva, oral fluid, tears, urine, interstitial fluid, synovial fluid, and cerebrospinal fluid. In some embodiments, the biological sample is blood. In some embodiments, the biological sample is blood, serum, plasma, or a blood fraction. In some embodiments, the blood is collected from a subject suspected of having a Lyme disease infection. In some embodiments, the blood is collected from a subject who is not suspected of having a Lyme disease infection.

In some embodiments, the subject is a human or a non-human animal. In some embodiments, the subject is a human. In some embodiments, the sample is collected from a subject suspected of having a disease caused by a pathogenic member of the bacterial genus Borrelia. In some embodiments, the sample is collected from a subject who is not suspected of having a disease caused by a pathogenic member of the bacterial genus Borrelia. In some embodiments, the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica. In some embodiments, the pathogenic member of the bacterial genus Borrelia is Borrelia burgdorferi.

In some embodiments, the first antigen is a peptide or protein comprising an amino acid sequence having at least 70% sequence identity to the amino acid sequence set forth in any one of SEQ ID NOs: 1-83. In some embodiments, the first antigen is a protein comprising an amino acid sequence having at least 70% sequence identity to the amino acid sequence set forth in any one of SEQ ID NO: 1-6. In some embodiments, the second antigen is a peptide or protein comprising an amino acid sequence having at least 70% sequence identity to the amino acid sequence set forth in any one of SEQ ID NOs: 1-83. In some embodiments, the second antigen is a peptide comprising an amino acid sequence having at least 70% sequence identity to the amino acid sequence set forth in any one of SEQ ID NOs: 7-83. In some embodiments, the sequence identity is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 100%.

In some embodiments, the detectable label is a luminescent label, an enzymatic or colorimetric label, or a radiometric label. In some embodiments, the luminescent label is a luminescent dye, a FRET label, or a fluorescent protein. In some embodiments, the luminescent label is a lanthanide, a fluorescent label, or an organic dye. In some embodiments, the colorimetric label is a horseradish peroxidase label or alkaline phosphatase label. In some embodiments, the colorimetric label is colloidal gold, colloidal carbon, a latex particle dyed with a visible dye, or any particle comprising a visible dye. In some embodiments, the detectable label is adapted to be detected by detecting a signal pulse emitted by the detectable label. In some embodiments, the detectable label is adapted to be detected by a substrate. In some embodiments, the substrate is selected from the group consisting of: ABTS, OPD, AmplexRed, DAB, AEC, TMB, Homovanillic Acid, or Luminol. In some embodiments, the substrate is 3,3′,5,5′-tetramethylbenzidine (TMB). In some embodiments, the substrate is Luminol.

In some embodiments, the vessel is adapted to receive at least a portion of the sample. In some embodiments, the vessel is a well. In some embodiments, the vessel is a well of a microplate comprising one or more wells.

In some embodiments, the presence of a signal is also indicative of the presence of the antibody in the sample. In some embodiments, the presence of a signal is suggestive of a Lyme disease infection in the subject. In some embodiments, the presence of a signal is indicative of Lyme disease-specific antibodies. In some embodiments, the presence of a signal is indicative of a previous or active Lyme disease infection in the subject. In some embodiments, the presence of a signal is indicative of an active Lyme disease infection in the subject.

In some embodiments, the first antigen-binding site and the second antigen-binding site are identical. In some embodiments, the first antigen-binding site and the second antigen-binding site share at least 90% sequence identity. In some embodiments, the first antigen-binding site and the second antigen-binding site share 91%, 92%, 93%, 94%, 95%, 96%, 97%, 98%, 99%, 99.5%, or 100% sequence identity. In some embodiments, the first antigen is capable of binding to the first antigen-binding site or the second antigen-binding site. In some embodiments, the second antigen is capable of binding to the first antigen-binding site or the second antigen-binding site.

Aspects of the present disclosure relate to a composition, comprising: an antibody derived from a sample; a first antigen bound to a first antigen-binding site; a second antigen bound to a second antigen-binding site; wherein: the second antigen is conjugated to a detectable label; and the first antigen and the second antigen are different.

In some embodiments, the first antigen is a recombinant protein. In some embodiments, the second antigen is a peptide derived from the recombinant protein. In some embodiments, the second antigen is a recombinant protein. In some embodiments, the first antigen is a peptide derived from the recombinant protein. In some embodiments, the first antigen is a first peptide and the second antigen is a second peptide.

In some embodiments, the first antigen is selected from a group consisting of: VlsE, C6, OspC, C10, BBK07, OppA2, and Decorin Binding Protein A. In some embodiments, the second antigen is selected from a group consisting of: VlsE, C6, OspC, C10, BBK07, OppA2, and Decorin Binding Protein A. In some embodiments, the first antigen is VlsE and the second antigen is C6. In some embodiments, the first antigen is C6 and the second antigen is VlsE. In some embodiments, the first antigen is OspC and the second antigen is C10. In some embodiments, the first antigen is C10 and the second antigen is OspC.

In some embodiments, sample is a biological sample. In some embodiments, the biological sample is selected from the group consisting of: saliva, oral fluid, tears, urine, interstitial fluid, synovial fluid, and cerebrospinal fluid. In some embodiments, the biological sample is blood. In some embodiments, the biological sample is blood, serum, plasma, or a blood fraction. In some embodiments, the blood is collected from a subject suspected of having a Lyme disease infection. In some embodiments, the blood is collected from a subject who is not suspected of having a Lyme disease infection.

In some embodiments, the subject is a human or a non-human animal. In some embodiments, the subject is a human. In some embodiments, the sample is collected from a subject suspected of having a disease caused by a pathogenic member of the bacterial genus Borrelia. In some embodiments, the sample is collected from a subject who is not suspected of having a disease caused by a pathogenic member of the bacterial genus Borrelia. In some embodiments, the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica. In some embodiments, the pathogenic member of the bacterial genus Borrelia is Borrelia burgdorferi.

In some embodiments, the first antigen is a peptide or protein comprising an amino acid sequence having at least 70% sequence identity to the amino acid sequence set forth in any one of SEQ ID NOs: 1-83. In some embodiments, the first antigen is a protein comprising an amino acid sequence having at least 70% sequence identity to the amino acid sequence set forth in any one of SEQ ID NO: 1-6. In some embodiments, the second antigen is a peptide or protein comprising an amino acid sequence having at least 70% sequence identity to the amino acid sequence set forth in any one of SEQ ID NOs: 1-83. In some embodiments, the second antigen is a peptide comprising an amino acid sequence having at least 70% sequence identity to the amino acid sequence set forth in any one of SEQ ID NOs: 7-83. In some embodiments, the sequence identity is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 100%.

In some embodiments, the detectable label is a luminescent label, an enzymatic or colorimetric label, or a radiometric label. In some embodiments, the luminescent label is a luminescent dye, a FRET label, or a fluorescent protein. In some embodiments, the luminescent label is a lanthanide, a fluorescent label, or an organic dye. In some embodiments, the colorimetric label is a horseradish peroxidase label or alkaline phosphatase label. In some embodiments, the colorimetric label is colloidal gold, colloidal carbon, a latex particle dyed with a visible dye, or any particle comprising a visible dye. In some embodiments, the detectable label is adapted to be detected by detecting a signal pulse emitted by the detectable label. In some embodiments, the detectable label is adapted to be detected by a substrate. In some embodiments, the substrate is selected from the group consisting of: ABTS, OPD, AmplexRed, DAB, AEC, TMB, Homovanillic Acid, or Luminol. In some embodiments, the substrate is 3,3′,5,5′-tetramethylbenzidine (TMB). In some embodiments, the substrate is Luminol.

In some embodiments, the presence of a signal is also indicative of the presence of the antibody in the sample. In some embodiments, the presence of a signal is suggestive of a Lyme disease infection in the subject. In some embodiments, the presence of a signal is indicative of Lyme disease-specific antibodies. In some embodiments, the presence of a signal is indicative of a previous or active Lyme disease infection in the subject. In some embodiments, the presence of a signal is indicative of an active Lyme disease infection in the subject.

In some embodiments, the first antigen-binding site and the second antigen-binding site are identical. In some embodiments, the first antigen-binding site and the second antigen-binding site share at least 90% sequence identity. In some embodiments, the first antigen-binding site and the second antigen-binding site share at least 91%, at least 92%, at least 93%, at least 94%, at least 95%, at least 96%, at least 97%, at least 98%, at least 99%, at least 99.5%, or 100% sequence identity. In some embodiments, the first antigen is capable of binding to the first antigen-binding site or the second antigen-binding site. In some embodiments, the second antigen is capable of binding to the first antigen-binding site or the second antigen-binding site.

Aspects of the present disclosure relate to the use of antigens to determine whether a particular antibody is present in a sample derived from a subject. In some embodiments, the antigen is any molecule that is able to bind to an antibody. In some embodiments, the antigen is a protein, a polysaccharide, a lipid, or another molecule. In some embodiments, the antigen is a protein. In some embodiments, the antigen is a recombinant protein. In some embodiments, the antigen is a peptide. In some embodiments, the antigen is a peptide derived from a protein or recombinant protein. In some embodiments, the antigen comprises an epitope. An epitope is a part of an antigen that binds to an antibody. In some embodiments, the epitope of the antigen binds to an antigen-binding site on an antibody. The antigen-binding site, or paratope, is a site on an antibody that binds to the epitope of an antigen. In some embodiments, an antibody comprising two antigen-binding sites bind to two identical antigens each comprising two identical epitopes. In some embodiments, an antibody comprising two antigen-binding sites bind to two identical antigens each comprising different epitopes. In some embodiments, an antibody comprising two antigen-binding sites bind to two different antigens each comprising identical epitopes. In some embodiments, an antibody comprising two antigen-binding sites bind to two different antigens each comprising different epitopes. In some embodiments, the epitope is a linear epitope. In some embodiments, a linear epitope is an epitope that is recognized by antibodies by its primary structure. In some embodiments, the epitope is a conformational epitope. In some embodiments, a conformational epitope is an epitope that is recognized by antibodies by its three-dimensional structure. In some embodiments, the antigen comprises an amino sequence as set forth in any one of SEQ ID NOs: 1-83. In some embodiments, the antigen comprises an amino acid sequence that is at least 70% identical to any one of SEQ ID NOs: 1-83. In some embodiments, the antigen comprises an amino sequence as set forth in any one of SEQ ID NOs: 1-6. In some embodiments, the antigen comprises an amino acid sequence that is at least 70% identical to any one of SEQ ID NOs: 1-6. In some embodiments, the antigen comprises an amino sequence as set forth in any one of SEQ ID NOs: 7-83. In some embodiments, the antigen comprises an amino acid sequence that is at least 70% identical to any one of SEQ ID NOs: 7-83. In some embodiments, the antigen is any one of the proteins or peptides described herein (e.g., VlsE, C6, OspC, C10, BBK07, OppA2, and Decorin Binding Protein A). In some embodiments, the antigen is derived from an infectious disease pathogen. In some embodiments, the antigen is derived from a pathogen that causes Lyme disease, HIV, Dengue, Chagas, SARS-CoV-2, or any other infectious disease. In some embodiments, the antigen is derived from Borrelia burgdorferi, Borrelia garinii, Borrelia afzelii, Human immunodeficiency virus 1, Human immunodeficiency virus 2, SARS-CoV-2, Dengue virus, or Trypanosoma cruzi.

In some embodiments, any of the methods, systems, microplates, kits, or compositions described herein can be used in a lateral flow immunoassay. As will be understood by a person having ordinary skill in the art, an exemplary lateral flow immunoassay is described in U.S. Pat. No. 7,745,228, the entirety of which is hereby incorporated by reference. A lateral flow immunoassay is described herein as a Hybrid Lateral Flow Immunoassay. The Hybrid Lateral Flow Immunoassay is, in some embodiments, an immunoassay for rapidly detecting the presence of a particular antibody in the blood serum of a subject. The immunoassay is prepared by generating a test line on a nitrocellulose membrane. In some embodiments, the test line comprises an antigen that is specific for the particular antibody. In some embodiments, separately, another antigen that is also specific for the particular antibody is incubated with the blood serum. In some embodiments, the antigen is conjugated to a first binding molecule (e.g., biotin). To begin the immunoassay test, the nitrocellulose membrane is dipped into a solution comprising the sample and the antigen conjugated to a first binding molecule. If the antigen recognizes the antibody, the antigen will form a conjugate with the antibody and move across the nitrocellulose membrane by capillary action. Once the antibody-antigen conjugate reaches the test line comprising the antigen at the test line, the antibody, if recognized by the antigen at the test line, will form a antigen-antibody-antigen cross-linked conjugate at the test line. In some embodiments, after a sufficient incubation time, a second binding molecule (e.g., streptavidin, NeutrAvidin) conjugated to a detectable label (e.g., a luminescent label, colloidal gold, Europium) is added to the test line. The second binding molecule will bind to the first binding molecule, if present at the test line, and the detectable label can be detected. The presence of a signal from the detectable label is indicative of the first binding molecule binding the second binding molecule, each antigen binding the antibody, and the presence of the antibody in the initial sample at a concentration sufficient for detection.

In some embodiments, an antigen that is specific for an antibody is immobilized to a test line on a solid substrate. In some embodiments, separately, another antigen that is also specific for the antibody is added to the solid substrate. In some embodiments, the non-immobilized antigen is conjugated to a first binding molecule (e.g., biotin). In some embodiments, the solid substrate is dried such that both antigens are dried onto the solid substrate. In some embodiments, a sample is added to the solid substrate. In some embodiments, the sample moves across the solid substrate via capillary action. In some embodiments, the non-immobilized antigen binds the antibody in the sample, if present, forming an antigen-antibody complex. In some embodiments, the antigen-antibody complex moves to the test line via capillary action. In some embodiments, the antigen-antibody complex binds to the immobilized antibody. forming an antigen-antibody-antigen crosslinked complex at the test line. In some embodiments, after a sufficient incubation time, a second binding molecule (e.g., streptavidin, NeutrAvidin) conjugated to a detectable label (e.g., a luminescent label, colloidal gold, Europium) is added to the test line. In some embodiments, the second binding molecule binds to the first binding molecule, if present at the test line, and the detectable label is detected. The presence of a signal from the detectable label is indicative of the first binding molecule binding the second binding molecule, each antigen binding the antibody, and the presence of the antibody in the initial sample at a concentration sufficient for detection. In some embodiments, the detectable label is conjugated to the immobilized antigen. In some embodiments, the detectable label is conjugated to the non-immobilized antigen. In some embodiments, the detectable label is conjugated to the first binding molecule. In some embodiments, the detectable label is conjugated to the second binding molecule. In some embodiments, the first binding molecule and the second binding molecule form a binding molecule pair. In some embodiments, the first binding molecule is conjugated to a peptide. In some embodiments, the first binding molecule is conjugated to the peptide during synthesis of the peptide. In some embodiments, the second binding molecule is conjugated to a peptide. In some embodiments, the second binding molecule is conjugated to the peptide during synthesis of the peptide. In some embodiments, the first binding molecule is conjugated to a natural or recombinant protein. In some embodiments, the first binding molecule is conjugated to the natural or recombinant protein during a separate conjugation reaction. In some embodiments, the second binding molecule is conjugated to a natural or recombinant protein. In some embodiments, the second binding molecule is conjugated to the natural or recombinant protein during a separate conjugation reaction.

In some embodiments, an antigen is immobilized on a solid substrate. In some embodiments, a solid substrate is a membrane. In some embodiments, a solid substrate is a nitrocellulose membrane. In some embodiments, a solid substrate is a Fusion 5 membrane. In some embodiments, the immobilized antigen is provided by the sequence of any one of SEQ ID NO: 1-83. In some embodiments, the immobilized antigen comprises an amino acid sequence that is at least 70% identical to any one of SEQ ID NOs: 1-83. In some embodiments, the immobilized antigen is immobilized at a concentration of 0.5 mg/mL, 1 mg/mL, 2 mg/mL, 3 mg/mL, 4 mg/mL, or 5 mg/mL. In some embodiments, the nitrocellulose membrane is coupled to a waste pad. In some embodiments, a non-immobilized antigen is added to the nitrocellulose membrane. In some embodiments, the non-immobilized antigen is biotinylated. In some embodiments, the non-immobilized antigen is PEGylated. In some embodiments, the non-immobilized antigen is biotinylated and PEGylated. In some embodiments, the non-immobilized antigen is provided by the sequence of any one of SEQ ID NO: 1-83. In some embodiments, the non-immobilized antigen comprises an amino acid sequence that is at least 70% identical to any one of SEQ ID NOs: 1-83. In some embodiments, the non-immobilized antigen is added to the nitrocellulose membrane at a concentration of 0.003 μg/mL, 0.004 μg/mL, 0.005 μg/mL, 0.01 μg/mL, 0.02 μg/mL, 0.03 μg/mL, 0.04 μg/mL, 0.05 μg/mL, 0.1 μg/mL, 0.2 μg/mL, or 0.3 μg/mL. In some embodiments, the non-immobilized antigen is added to the nitrocellulose membrane at a concentration of 0.01 μg/mL. In some embodiments, the non-immobilized antigen is incubated with subject blood serum comprising antibodies prior to being added to the nitrocellulose membrane. In some embodiments, the non-immobilized antigen binds to a first antigen-binding site on the antibody. In some embodiments, the binding is reversible. In some embodiments, the binding is irreversible. Without wishing to be bound by any theory, the inventors believe that the non-immobilized antigen travels on the nitrocellulose membrane by capillary action and interacts with the test strip. In some embodiments, as the non-immobilized antigen bound to the first antigen-binding site on the antibody travels on the nitrocellulose membrane, a second antigen-binding site on the antibody is conjugated to the immobilized antigen, resulting in a cross-linked antibody. In some embodiments, the antibody becomes cross-linked after 1 minute, 2 minutes, 3 minutes, 4 minutes, 5 minutes, 10 minutes, 15 minutes, 20 minutes, or 30 minutes after the non-immobilized antigen conjugated to the first antigen-binding site on the antibody is added to the nitrocellulose membrane. In some embodiments, a detectable label is added to the nitrocellulose membrane. In some embodiments, the detectable label is any detectable label described herein. In some embodiments, the detectable label is colloidal gold. In some embodiments, the detectable label is Europium. In some embodiments, the detectable label is bound to any avidin protein described herein. In some embodiments, the avidin protein is streptavidin. In some embodiments, the avidin protein is NeutrAvidin. In some embodiments, detection of the detectable label above a cut-off value is indicative of the presence of antibodies associated with any infectious disease described herein.

Potential limitations of diagnostic assays include false positive results obtained from samples derived from vaccinated subjects. One such limitation is detection of antibodies produced by a subject in response to a vaccine (as opposed to antibodies produced in response to an infection) as a positive result. For example, Valneva has reported that its VLA15 is expecting successful completion of Phase 3 clinical trials in the coming years (Czarnota et al., Journal of Education, Health and Sport. 2022 August; 12 (8): 1164-1169). VLA15 appears to be the only Lyme disease vaccine candidate currently in clinical development, but its potential success suggests an impending increase in the availability of Lyme disease vaccines and the number of subjects across the world who will be vaccinated against Lyme disease. A diagnostic assay for Lyme disease that cross-reacts with antibodies elicited by a Lyme disease vaccine could incorrectly indicate that a subject who is vaccinated against Lyme disease but who is uninfected has (or recently had) a Lyme disease infection. False positive results of this nature would mean the diagnostic assay would be unable to differentiate between a subject who has (or recently had) a Lyme disease infection and a subject who is vaccinated but uninfected. The Hybrid ELISA assay of the present disclosure provides an additional advantage in that it relies on antigens different from those used in VLA15, and is able to distinguish between antibodies produced in response to the VLA15 vaccine and those produced from a true Lyme disease infection. This is because the Hybrid ELISA assay of the present disclosure relies on the combination of VlsE protein and C6 peptide, while the VLA15 vaccine antigen relies on the OspA protein, the presence of which will not produce a positive result in the disclosed assay. Moreover, the combination of VlsE protein and C6 peptides ensures that antibodies produced in response to the VLA15 vaccine will not trigger a positive result. Accordingly, in some embodiments, the Hybrid ELISA assay disclosed herein does not cross-react with antibodies in a sample derived from a vaccinated but uninfected subject. In some embodiments, the Hybrid ELISA assay disclosed herein does not cross-react with antigens in a sample derived from a vaccinated but uninfected subject. In some embodiments, the Hybrid ELISA assay disclosed herein does not detect antibodies in a sample derived from a vaccinated but uninfected subject. In some embodiments, the Hybrid ELISA assay disclosed herein does not detect antigens in a sample derived from a vaccinated but uninfected subject. In some embodiments, the Hybrid ELISA assay disclosed herein does not provide a false positive result due to cross reactivity with antibodies in a sample derived from a vaccinated but uninfected subject. In some embodiments, the Hybrid ELISA assay disclosed herein does not provide a false positive result due to cross reactivity with antigens in a sample derived from a vaccinated but uninfected subject. In some embodiments, the vaccinated subject received a vaccine about 1 minute, about 5 minutes, about 10 minutes, about 20 minutes, about 30 minutes, about 1 hour, about 2 hours, about 10 hours, about 12 hours, about 1 day, about 2 days, about 5 days, about 1 week, about 2 weeks, about 3 weeks, about 1 month, about 6 months, about 1 year, about 5 years or about 10 years prior to submitting a sample for a Hybrid ELISA diagnostic test according to the present disclosure. In some embodiments, the vaccine does not comprise the C6 peptide. In some embodiments, the vaccine does not comprises the VlsE protein. In some embodiments, the vaccine does not comprises the C6 peptide or the VlsE protein. In some embodiments, the vaccine comprises the OspA protein.

One skilled in the art can, based on the above description, utilize the present disclosure to its fullest extent. The following specific embodiments are, therefore, merely illustrative, and are not limitative of the remainder of the disclosure in any way whatsoever. All publications cited in the present application are incorporated by reference for the purposes or subject matter referenced in this disclosure.

### EXAMPLES

### Example 1. Evaluation of ELISA Assays for Detection of Lyme Disease

Bridge assays using either the C6 peptide or the VlsE protein as antigens were evaluated. In the bridge assay format (FIG. 1), the target antigen is immobilized on a solid substrate, and detection is accomplished by the addition of the same antigen, conjugated to a label. Antigen-specific antibodies thereby have the opportunity to bind the same antigen at both (for IgG) or multiple (for IgM) binding sites, whereby the label is effectively cross-linked to the solid phase. A fraction of antibodies will bind the soluble antigen conjugated to the label at both binding sites (if IgG), and will consequently not bind to the solid phase, escaping detection.

In practice, this loss can be compensated by appropriate optimization of the stoichiometry of the two antigen conjugates. The advantages of the bridge format are that a secondary antibody conjugate is not necessary for serum antibody detection. Eliminating the secondary antibody generally eliminates the associated non-specific reactivity resulting in higher assay specificity. The assay can be reduced to near-homogeneous format, as all components other than the enzyme substrate can be added simultaneously. Secondly, with low non-specific binding, serum can be assayed at low dilution or even neat, allowing higher analytical sensitivity. Additionally, the bridge assay allows antibody binding and detection independent of antibody class or host species. This may be an advantage where it is desirable to detect antibodies of all classes, or to use the same assay for e.g. humans and non-human reservoir species. Bridge assays have been used successfully in ELISA format (e.g., FDA-approved Anthrax ELISA previously developed by the Principal Investigator (U.S. Pat. No. 7,262,019)) and lateral flow format (e.g., Anthrax (Biagini et al., Clin Vaccine Immunol. 2006 May; 13 (5): 541-6), Alere HIV Determine (Alere et al., Combo Package Insert)).

Preliminary results demonstrated that C6- or VlsE-specific antibodies could be detected with relatively high sensitivity, but the level of non-specificity remained unacceptably high; importantly, the individual falsely reactive samples differed between the C6 and VlsE-based assays. This observation was significant in light of recent studies which a serial testing algorithm comprising a whole cell sonicate or VlsE ELISA as first step, followed by a C6 ELISA as second step, yielded higher overall specificity than either ELISA alone, because of the differing specificities of the antigens in each step; sensitivity was only modestly decreased, yielding a modified ELISA-based two-tier protocol which equaled the conventional version in specificity and outperformed it in sensitivity (Branda et al., Clin Infect Dis. 2017; 64 (8): 1074-80; Branda et al., Clin Infect Dis. 2011; Branda et al., Clin Infect Dis. 2018 December; 66 (7): 1133-9).

The differing reactivity of C6 and VlsE ELISAs was confirmed with non-specific sera, finding no overlap on a challenge panel comprising negative controls exhibiting high reactivity (Table 1). Table 1 demonstrates the false positive results in C6 ELISA and VlsE ELISA among a subset of negative control sera (n=55) selected for high OD values. None of the 8 false positive sera were detected by both ELISAs, indicating complementarity between assays. Of the 55 total sera, 10.9% were C6 positive and VlsE negative, while 3.6% were C6 negative and VlsE positive.

TABLE 1
False positive results in C6 ELISA and VlsE ELISA

| | VlsE Positive | VlsE Negative |
|---|---|---|
| C6 Positive | 0 | 6 |
| C6 Negative | 2 | 47 |

### Example 2. Development of Hybrid ELISA Based on VlsE Protein and C6 Peptide Antigens

### Serum Samples

Panels of well-characterized Lyme disease sera and controls were acquired from a variety of sources, comprising over 250 Lyme sera, 720 sera from healthy controls and 43 sera from patients with other disease conditions (Table 2 and Table 3). “Confirmed Lyme disease sera” comprised samples from patients with clinically diagnosed Lyme disease, either based on presence of physician-documented erythema migrans or culture or PCR positive results, independent of serologic status in cases of early-stage disease, or based on two-tier serologic results for later-stage disease.

TABLE 2
Summary of Lyme disease serum samples acquired
in Examples 1-3 for evaluation of Hybrid ELISA

| | | | # | # Two- | |
|---|---|---|---|---|---|
| | | # | Conva- | Tier | |
| Source | Number | Acute | lescent | Positive | Description |
| New York | 50 | 37 | 13 | 15 | Clinically diagnosed |
| Medical | | | | | Lyme disease patients |
| College | | | | | |
| NIH/NIAID | 100 | ND | ND | 10 | Clinically diagnosed |
| | | | | | Lyme disease patients |
| CDC | 12 | 8 | 4 | 12 | Clinically diagnosed |
| | | | | | Lyme disease patients |
| Lyme | 58 | 55 | 3 | 28 | Clinically diagnosed |
| Disease | | | | | Lyme disease patients |
| Biobank | | | | | |
| Boca | 4 | 2 | 2 | 4 | Clinically diagnosed |
| Biolistics | | | | | Lyme disease patients |
| MRN | 25 | ND | ND | 25 | Two-tier positive |
| | | | | | samples |
| Plasma | 8 | 3 | 5 | 8 | Two-tier positive |
| Services | | | | | samples |
| Group | | | | | |
| TOTAL | 257 | 105 | 27 | 102 | |

TABLE 3
Summary of control serum samples acquired in
Examples 1-3 for evaluation of Hybrid ELISA

| Source | Number | Description |
|---|---|---|
| Creative Testing Solutions | 100 | Healthy blood donors |
| American Red Cross | 200 | Healthy blood donors |
| CDC | 8 | Healthy controls |
| Lyme Disease Biobank | 7 | Healthy controls |
| Plasma Services Group | 5 | Healthy blood donors |
| Internal Collection | 400 | Healthy blood donors |
| CDC | 12 | Other disease conditions |
| Boca Biolistics | 45 | Other disease conditions |
| TOTAL Healthy Controls | 720 | |
| TOTAL Other Disease | 57 | |
| Conditions | | |

### Assay Development

The Hybrid Lyme ELISA is based on the principle that an individual multivalent antibody can bind the C6 peptide at one binding site and the homologous sequence within the recombinant VlsE protein at another binding site (FIG. 2). Antibodies that bind solely to one antigen will not be detected. In certain embodiments, serum antibodies are simultaneously incubated with a mixture of VlsE (immobilized on the microplate well) and C6-HRP conjugate in solution in a single binding step. In these embodiments, the binding preference of antibodies for one vs. the other antigen is affected by the corresponding relative affinities. To maximize the proportion of antibodies binding to both C6 and VlsE antigens, the stoichiometry of the VlsE protein and C6-HRP conjugate can be optimized. In addition, to account for the possibility that the conformation of the C6 peptide can influence antibody binding affinity unequally across serum antibodies, a mixture of two different C6-HRP conjugates, in which the method of conjugation and linker chemistry differed, can provide for improved sensitivity of detection.

One C6-HRP conjugate was generated by binding N-terminal biotinylated C6 peptide to an HRP-Streptavidin conjugate (obtained from a commercial supplier) followed by size exclusion chromatography to remove unbound peptide. A second conjugate was generated by direct crosslinking of the C6 peptide to HRP; this approach used a C6 peptide with N-terminal cysteine added (there are no other cysteines in the C6 sequence), which was conjugated to maleimide-activated HRP using a commercial crosslinking kit. Excess, unconjugated peptide was removed by chromatography. The two C6 peptide-HRP conjugates showed complementary reactivity with Lyme serum samples, some samples being detected only by one conjugate and some by the other. The concentrations and stoichiometry of these components in the conjugate mixture were likewise optimized.

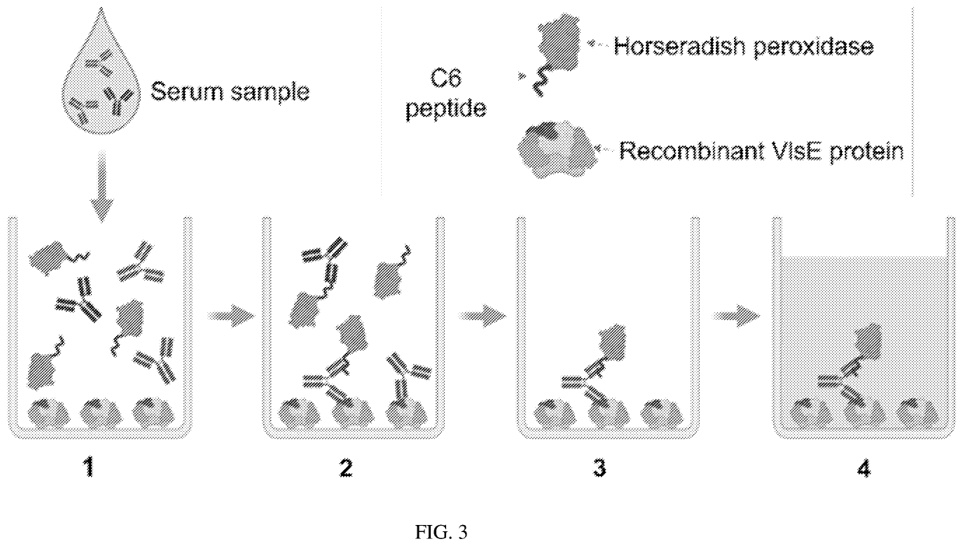

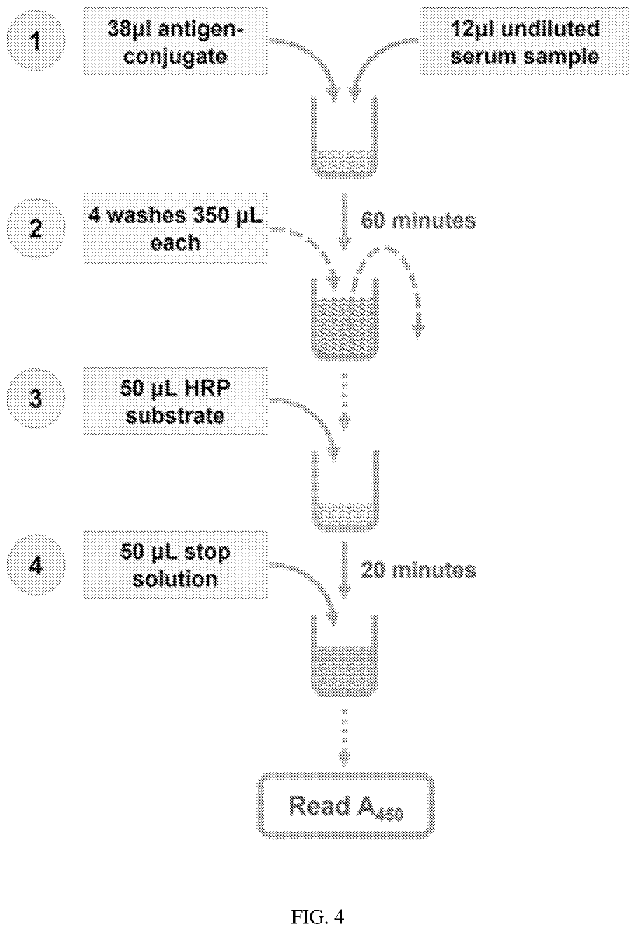

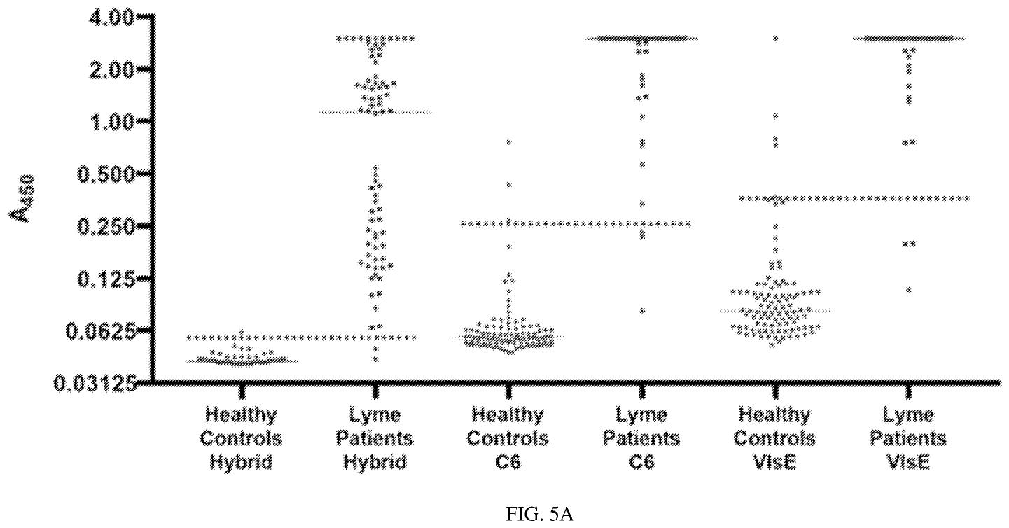

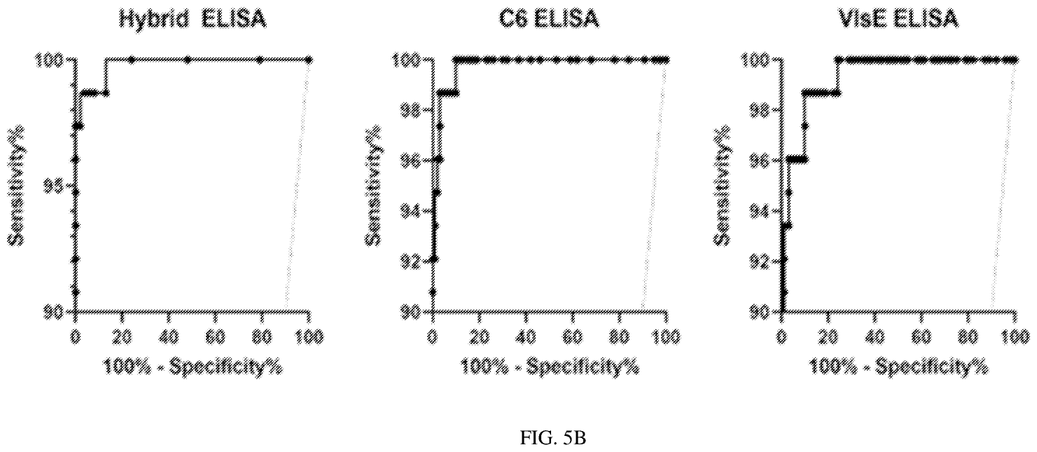

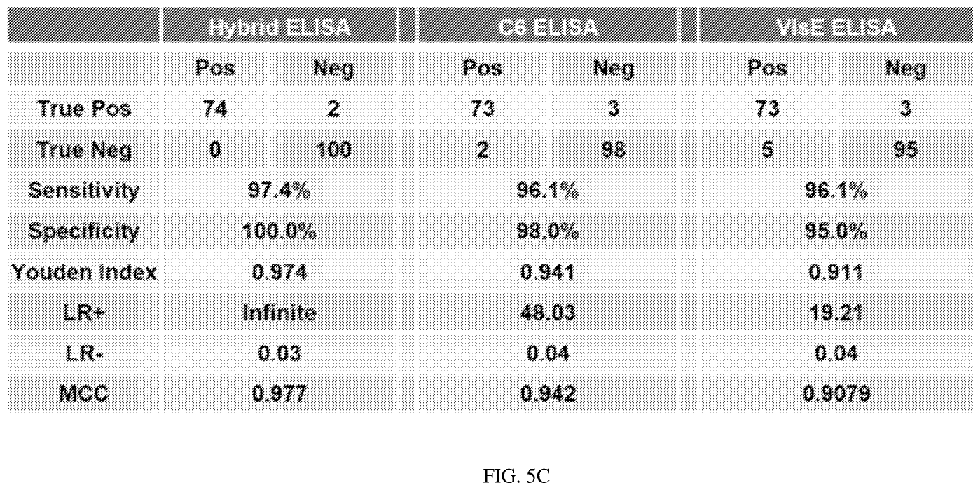

The resultant Hybrid ELISA protocol was simpler and faster than most conventional ELISA protocols, with only four main steps (FIG. 3 and FIG. 4). The serum sample (neat) and C6 peptide-HRP conjugate solution (provided prediluted in a ready-to-use solution) were added simultaneously to the ELISA well, incubated, and unbound material was removed by four buffer washes. The HRP substrate was then added, allowed to develop during a brief incubation, and the reaction quenched with stop solution. For colorimetric detection, tetramethylbenzidine solution was used as substrate, with readout at 450 nm using a conventional ELISA microplate reader (see FIG. 5A). Chemiluminescent detection using a commercial luminol-based HRP substrate was also evaluated.

The entire assay was carried out in less than 90 minutes. To interpret the result, the absorbance reading is divided by the cut-off to yield an index value. Index values <0.9 are interpreted as negative, 0.9<index value <1.1 as equivocal, and >1.1 as positive.

### Example 3. Demonstration of Sensitivity and Specificity of Hybrid ELISA with Well-Characterized Lyme-Positive, Negative, and Cross-Reactive Serum Samples

The sensitivity and specificity of the Hybrid ELISA as a single-tier test were compared with the sensitivity and specificity of two-tier testing either based on archival data where they were available, or generated for this study using an FDA-approved whole-cell sonicate ELISA and FDA-approved recombinant immunoblot kit. The Hybrid ELISA was also compared with separate ELISAs based on either the C6 peptide or the recombinant VlsE protein in conventional indirect ELISA format (both developed separately and validated). Lastly, Hybrid ELISA performance as a single-tier test was compared with the two-tier protocol comprising Hybrid ELISA as the first step and IgG and IgM immunoblots as the second step.

### Cut-Off

The cut-offs for retrospective interpretation of the Hybrid ELISA, C6 ELISA and VlsE ELISA were selected to yield the optimal combination of sensitivity and specificity based on ROC data using GraphPad Prism. ROC (Receiver Operating Characteristic) is a data analysis technique that illustrates the diagnostic ability of a binary classifier system as its discrimination threshold is varied. Subsequent modeling showed that the ROC-based cut-off for the Hybrid ELISA, with associated performance values, could be replicated by adding a fixed factor to the absorbance of the negative control. This cut-off approach was used in the analyses below. Commercial embodiments can be implemented that substitute a separate calibrator serum (e.g., included as a component of the kit) which exhibits reactivity similar to the negative control.

In the analyses of sensitivity and specificity below, equivocal (indeterminate) results were combined with positive results and interpreted as positive. About 3% of the 136 Lyme patient sera in total that were tested on Hybrid ELISA yielded a result in the “equivocal” range (0.9<LI<1.1), but none of the 627 control sera tested yielded an equivocal result.

Comparison with C6 ELISA and VlsE ELISA

In an initial comparison of the Hybrid ELISA with a C6 ELISA and a VlsE ELISA on a set of 76 two-tier positive Lyme patients and 100 healthy controls, the Hybrid ELISA demonstrated 97.4% sensitivity and 100% specificity, higher than both of the other individual assays (FIGS. 5A-C). The higher stringency of binding inherent in the Hybrid ELISAs principle of simultaneously binding synthetic peptide and recombinant protein epitopes results in negligible spurious or false reactivity, and the lack of a traditional second antibody-enzyme conjugate substantially eliminates background reactivity. These characteristics which are intrinsic to the Hybrid ELISA format allow for a relatively low cut-off value in comparison with conventional indirect ELISAs. ROC analysis indicated the feasibility of a Hybrid ELISA cut-off that would provide comparatively higher specificity than possible for the C6 and VlsE ELISAs, while retaining high sensitivity (FIG. 5B). As a corollary, the reactivity of true positive sera is distributed across a wider range than seen in the other two conventional indirect ELISAs, with proportionally more serum samples detected at lower reactivity levels. As Lyme ELISAs are qualitative tests, intended to deliver a positive or negative result, the absolute value of the ELISA absorbance (or the signal/cut-off, S/CO) is not significant for interpretation of the result. In the current two-tier algorithm, all samples showing positive or equivocal ELISA results are re-tested by immunoblot, which is confirmatory. In fact, the Hybrid ELISA detected two serum samples with low levels of reactivity from Lyme patients which were collected within 7 days of onset of symptoms, and which were negative on the C6 ELISA and VlsE ELISA.

Furthermore, the combination of sensitivity and specificity offered by the Hybrid ELISA could not be achieved by the C6 ELISA or VlsE ELISA merely by changing the cut-off. Using the same ROC data as in FIGS. 5A-C, if the cut-offs of C6 ELISA and VlsE ELISA as well as that of the Hybrid ELISA were modified to yield 99% specificity, the sensitivities of the two other ELISAs were both less by several percentage points than the 97.4% shown by the Hybrid ELISA (Table 4; note that sensitivity of Hybrid ELISA was 97.4% at specificity values ≥98%). Similarly, if the same cut-offs were modified to yield 97.4% sensitivity for C6 ELISA and VlsE ELISA, equal to that of the Hybrid ELISA, then the specificities of the C6 and VlsE ELISAs were lower by several percentage points than the 100% shown by the Hybrid ELISA.

TABLE 4
Comparative sensitivity and specificity of ELISAs
at equivalent specificity or sensitivity as calculated
from the same ROC data used in FIGs. 5A-C

| | | |
|---|---|---|
| Hybrid ELISA | VlsE ELISA | C6 ELISA |

Sensitivity

| | | | |
|---|---|---|---|
| All assays at | 97.4% | 93.4% | 94.7% |
| specificity = 99.0% | | | |

Specificity

| | | | |
|---|---|---|---|
| All assays at | 100.0% | 90.0% | 97.0% |
| sensitivity = 97.4% | | | |

Three statistical measures of diagnostic test performance were applied for comparative evaluation of Hybrid ELISA vs. C6 ELISA and VlsE ELISA (FIG. 5C). The Youden index or J statistic, a statistic which provides a summary measurement of the ROC curve and is an indicator of overall test accuracy (Shan et al., PLOS One. 2015; 10 (7): 1-19; Marcus et al., Biom J. 2008; 23 (3): 419-30), was higher for Hybrid ELISA than for C6 and VlsE ELISAs. The positive likelihood ratio (LR+), a measurement of the post-test probability of disease, or the test's ability to correctly identify true positive cases independent of prevalence (Baratloo et al., Emerg (Tehran, Iran). 2015; 3 (4): 170-1), was infinite due to the 100% specificity registered by the Hybrid ELISA, but even at 99% specificity for Hybrid ELISA, it exceeded that of the C6 ELISA by two-fold and the VlsE ELISA by over four-fold. Conversely, the negative likelihood ratio (LR−), or the test's ability to exclude true negative cases, was lowest for the Hybrid ELISA. While sensitivity and specificity are measurements of retrospective test performance on defined positive and negative patient groups, the likelihood ratio is more useful as an indicator of prospective test performance in the clinical setting where the diagnosis has not yet been made. Lastly, the Matthews Correlation Coefficient (MCC) measures the ability of a test to correctly identify both true positives and true negatives, independent of the size of each group (Chicco et al., BMC Genomics. 2020; 21 (1): 1-13; Chicco et al., BioData Min. 2021; 14:1-22). While MCC is not as commonly applied to diagnostic tests as the other statistics, it is a powerful indicator of overall test accuracy. The MCC value for Hybrid ELISA was likewise higher than that for either C6 or VlsE ELISA.

### Cross-Reactivity

The Hybrid ELISA showed 100% specificity when tested on a panel of 57 sera from patients with a variety of other disease conditions which could lead to cross-reactivity (Table 5). Cross-reactivity was also evaluated on serum samples from patients with two other tick-borne infections: Borrelia miyamotoi and Babesia microti. Four out of five B. miyamotoi sera tested positive on the Hybrid ELISA, presumably due to the strong homology between the C6 peptide sequence of B. burgdorferi and that of the related B. miyamotoi, consistent with observations in prior studies; however, co-infection with B. burgdorferi cannot be ruled out. One out of seven B. microti sera was found equivocal on the Hybrid ELISA, and likewise reactive on a whole cell sonicate Lyme ELISA and the C6 and VlsE ELISAs, suggesting that the patient was or had been co-infected with B. burgdorferi, a likely occurrence due to the shared tick vector that transmits both infections. These results suggested the Hybrid ELISA platform can be used to detect infectious diseases beyond Lyme Disease.

TABLE 5
Evaluation of Hybrid ELISA specificity on cross-reactivity panel

| Disease Condition | # Positive/#Samples |
|---|---|
| Syphilis | 0/7 |
| Influenza | 0/4 |
| H. pylori | 0/5 |
| CMV | 0/5 |
| EBV | 0/5 |
| RA | 0/6 |
| MS | 0/7 |
| HIV-1/2 | 0/8 |
| COVID-19 | 0/6 |
| Fibromyalgia | 0/2 |
| Periodontitis | 0/2 |
| Total | 0/57 |

*CMV = cytomegalovirus, EBV = Epstein Barr virus, RA = Rheumatoid Arthritis, MS = Multiple Sclerosis

### Specificity

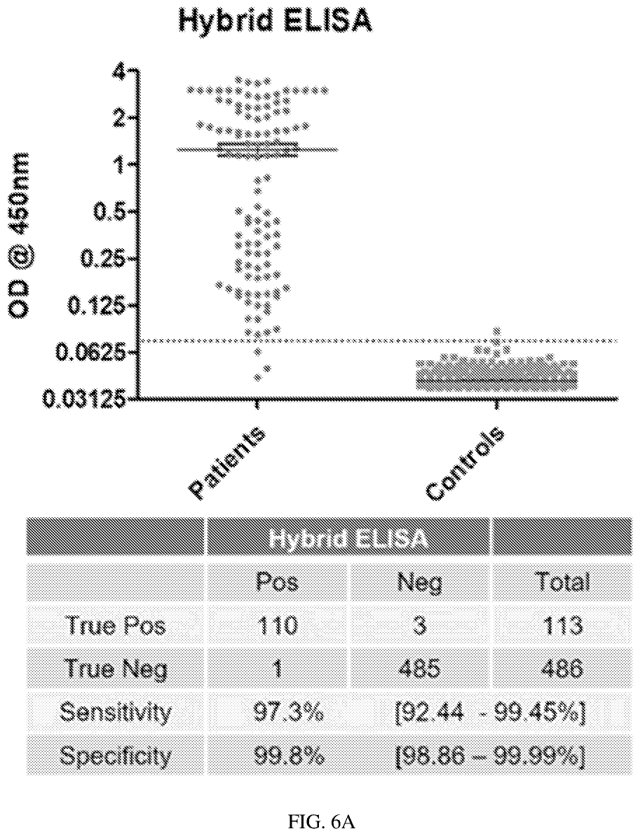

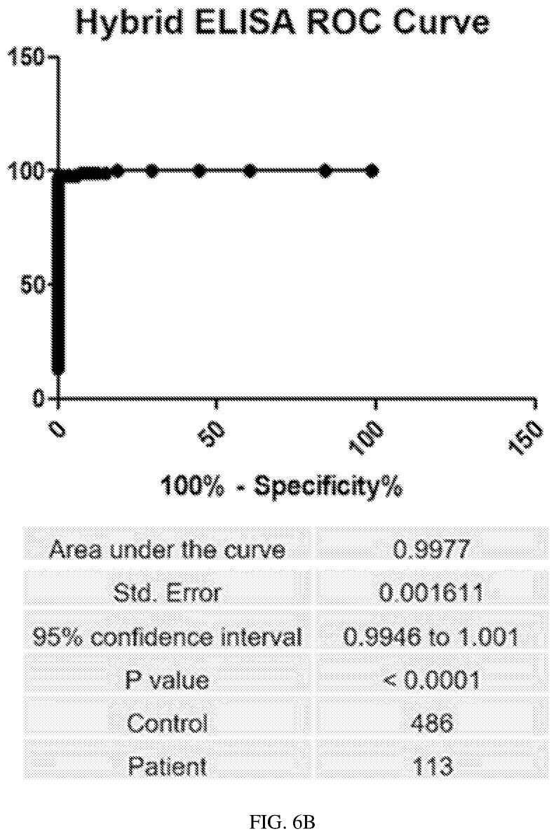

Testing an expanded panel of 486 healthy control sera from non-endemic regions (New Mexico and Oklahoma) in parallel with 113 sera from confirmed Lyme disease patients (sera from Table 2 and Table 3 that were available at the time of testing) yielded 97.3% sensitivity and 99.8% specificity (FIGS. 6A-B).

Comparison with Standard Two-Tier Testing (STTT)

The Hybrid ELISA was evaluated on the R67 Research Panel provided by the CDC, comprising 12 Lyme patient sera including both early and late stages and 20 controls including healthy individuals and patients with other disease conditions. The Hybrid ELISA detected 10/12 Lyme patient sera, while 8/12 were detected by standard two-tier testing using FDA-approved ELISA and immunoblot kits (archived results provided by CDC) (Table 6). The two sera detected by Hybrid ELISA that were missed by two-tier testing were both from acute stage erythema migrans patients. Two other sera that were missed by both Hybrid ELISA and two-tier testing were negative on whole cell sonicate ELISA, C6 ELISA, and immunoblots. The evaluation was next expanded to include a total of 109 sera from confirmed Lyme patients and 627 sera from controls including both healthy individuals and patients with other disease conditions, all samples listed in Table 2 and Table 3 above. Lyme patient sera included a subset of 67 sera from early stage patients ≤30 days from disease onset or diagnosed based on presence of erythema migrans alone, independent of serologic results, and the remaining 42 from presumed later stage patients that did not have erythema migrans but were positive via two-tier testing.

The Hybrid ELISA proved significantly (p<0.05) more sensitive than standard two-tier testing in the subset of early stage sera that were defined by EM independent of serology, detecting 25% (18) more than STTT (Table 7 and Table 8). Of equal importance, the Hybrid ELISA demonstrated 99.7% concordance with two-tier testing results among the 627 control sera, with p=0.5 indicating no significant difference between results (Table 9). Seven serum samples obtained from healthy individuals were found positive by both Hybrid ELISA and STTT; whether due to exposure to B. burgdorferi during travel or otherwise is unknown, but having tested positive by STTT, these samples are excluded from the “true negative” group by definition. Only two serum samples were found positive by Hybrid ELISA but negative by STTT. Overall, the Hybrid ELISA yielded significantly greater sensitivity than STTT while maintaining equivalent specificity, especially with respect to early stage Lyme sera. The Youden index for Hybrid ELISA was significantly higher than for STTT, with the difference further magnified for early stage Lyme sera. These results support the conclusion that the Hybrid ELISA is not inferior to STTT, and in fact appears to offer a net improvement in performance.

The sensitivity and specificity of the Hybrid ELISA assay disclosed herein was further compared with the MTTT protocol using Zeus ELISA kits approved by the FDA for MTTT use. The Zeus MTTT protocol uses a VlsE/pepC10 ELISA as the first step and separated IgG and IgM whole-cell sonicate ELISAs as the second step. To reveal differences where assay sensitivity is most challenged, the same set of 67 early-stage Lyme sera as previously tested against STTT (Table 7) was tested again. Similarly, the same set of 57 sera from patients with other disease conditions as tested previously by the Hybrid ELISA assay disclosed herein (Table 5) was tested again. These sera were supplemented by 54 sera from normal, healthy blood donors. Results from these tests showed the Hybrid Lyme ELISA assay disclosed herein to be more sensitive than both the first and second step Zeus ELISA, with a statistically significant 12% advantage over MTTT in final sensitivity (Table 10). Likewise, the Hybrid Lyme ELISA assay disclosed herein was more specific than both the first and second step Zeus ELISAS, with a statistically significant advantage over the first step ELISA (Table 10). The Youden index for each assay further reflects the superior overall performance of the Hybrid Lyme ELISA assay disclosed herein. The Youden index (J statistic) was calculated as [sensitivity+specificity−1]. Significance of the difference in sensitivity and specificity between the Hybrid ELISA assay disclosed herein and MTTT was calculated by McNemar's Test.

The sensitivity of detection was further stratified by Hybrid ELISA vs. the major clinical manifestation of disease, grouping 87 Lyme serum samples for which this information was available into 3 categories-erythema migrans, Neuroborreliosis and Lyme Arthritis (Table 11). While Neuroborreliosis samples appeared to be detected with slightly higher sensitivity than Lyme Arthritis samples, which were in turn slightly higher than EM samples, the differences were not statistically significant given the relatively small sample sizes.

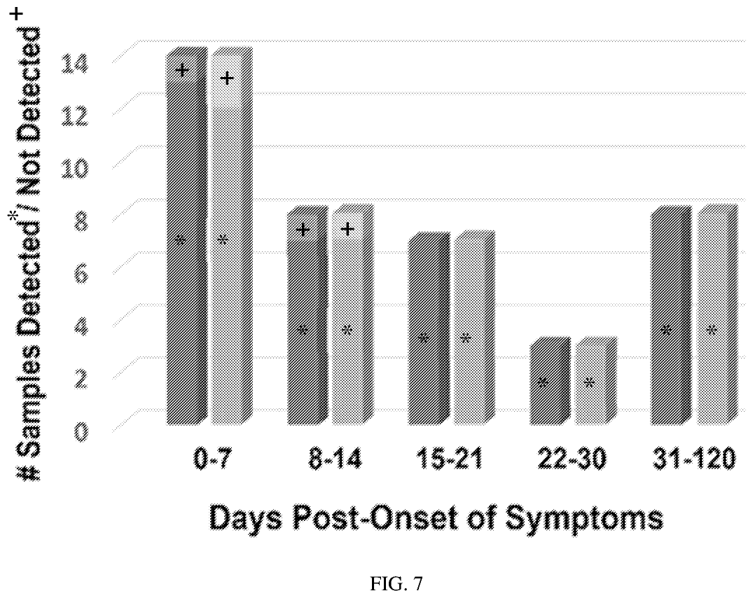

With respect to the sensitivity of detection vs. timing, the Hybrid ELISA vs. C6 ELISA were compared on a subset of early Lyme serum samples for which the dates of sample draw and of onset of symptoms were known. The Hybrid ELISA proved slightly more sensitive than C6 ELISA, detecting 13/14 serum samples drawn within one week of disease onset, vs. 12/14 for C6 ELISA (FIG. 7). The two ELISAs yielded identical sensitivity for samples drawn at later time points. While the sample size was too limited to draw statistical conclusions, these results suggest that the Hybrid ELISA is capable of detecting Lyme sera at very early time points with high sensitivity.

Next, it was determined whether any improvement in performance could be achieved by pairing the Hybrid ELISA with an immunoblot as a second tier test, emulating the conventional standard two tier testing protocol. The Hybrid ELISA-Immunoblot two tier testing protocol yielded no significant difference (p>0.05) in either sensitivity or specificity vs. the Hybrid ELISA alone when evaluated on panels of 93 Lyme patient sera, including 47 early stage Lyme sera, and 627 negative control sera (Table 12 and Table 13).

TABLE 6
Evaluation of CDC Research Panel I on Hybrid ELISA
vs. Two-Tier Testing via ELISA and immunoblot

| | Hybrid | Hybrid | | | |
|---|---|---|---|---|---|
| | ELISA | ELISA | Two-Tier | Two-Tier | |
| | Positive | Negative | Positive | Negative | Total |
| Clinically | 10 | 2 | 8 | 4 | 12 |
| Positive | | | | | |
| Clinically | 0 | 20 | 0 | 20 | 20 |
| Negative | | | | | |
| Total | 10 | 22 | 8 | 24 | 32 |

TABLE 7
Sensitivity and specificity of Hybrid ELISA
vs. Standard two-tier testing (STTT)

| | Later stage Lyme sera | | Early stage Lyme sera | |
|---|---|---|---|---|
| | Hybrid ELISA | STTT | Hybrid ELISA | STTT |
| Sensitivity | 100% | 100% | 94.0% | 68/7% |
| True positives | 42 | | 67 | |
| (n = 109) | | | | |
| | Healthy control sera | | Healthy control sera | |
| Specificity | 99.7% | 100% | 99.7% | 100% |
| True negatives | 620 | | 620 | |
| (n = 620) | | | | |
| Youden Index | 0.96 | 0.81 | 0.94 | 0.69 |

TABLE 8
Hybrid ELISA vs Standard two-tier testing (STTT) results
for 109 confirmed Lyme disease patient sera

| | Hybrid ELISA | Hybrid ELISA | |
|---|---|---|---|
| | Positive | Negative | Total |
| STTT Positive | 87 | 1 | 88 |
| STTT Negative | 18 | 3 | 21 |
| Total | 105 | 4 | 109 |

| | |
|---|---|
| McNemar's two-tailed p-value | 0.000076 |

TABLE 9
Hybrid ELISA vs Standard two-tier testing (STTT)
results for 627 negative control sera

| | Hybrid ELISA | Hybrid ELISA | |
|---|---|---|---|
| | Positive | Negative | Total |
| STTT Positive | 7 | 0 | 7 |
| STTT Negative | 2 | 618 | 620 |
| Total | 0 | 618 | 627 |

| | |
|---|---|
| McNemar's two-tailed p-value | 0.5 |

TABLE 10
Sensitivity and specificity of Hybrid ELISA
vs. Modified Two-Tier Testing (MTTT)

| | | | MTTT | MTTT |
|---|---|---|---|---|
| | | Hybrid | First | Second |
| Sample Type | Parameter | ELISA | Step | Step |
| Early-stage | Sensitivity | 94% | 91% | 82% |
| Lyme sera | (%, n) | (63) | (61) | (55) |
| (n = 67) | | | | |
| Healthy control | Specificity | 100% | 96% | 98% |
| and other | (%, n) | (111) | (107) | (109) |
| disease | | | | |
| conditions | | | | |
| (n = 111) | | | | |
| | Youden Index | 0.940 | 0.875 | 0.803 |

P (McNemar's)

| | | |
|---|---|---|
| Sensitivity | Hybrid ELISA vs. MTTT First Step | P = 0.414 |
| | Hybrid ELISA vs. MTTT Second Step | P = 0.011 |
| Specificity | Hybrid ELISA vs. MTTT First Step | P = 0.046 |
| | Hybrid ELISA vs. MTTT Second Step | P = 0.157 |

TABLE 11
Detection of Lyme sera in Hybrid ELISA vs. disease manifestation

| | % (# Total) Detected in Hybrid ELISA | |
|---|---|---|
| Erythema migrans (EM) | 94.44% | 51/54 |
| Neuroborreliosis | 100.00% | 7/7 |
| Lyme Arthritis | 96.15% | 25/26 |
| Total | | 87 |

TABLE 12
Hybrid ELISA One-Tier vs. Hybrid ELISA + Immunoblot Two-
Tier (HBTT) Testing results for 93 Lyme disease patient sera

| | Hybrid ELISA | Hybrid ELISA | |
|---|---|---|---|
| | Positive | Negative | Total |
| HB TT Positive | 86 | 0 | 86 |
| HB TT Negative | 1 | 6 | 7 |
| Total | 87 | 6 | 93 |

| | |
|---|---|
| McNemar's two-tailed p-value | 1 |

TABLE 13
Hybrid ELISA One-Tier vs. Hybrid ELISA + Immunoblot
Two-Tier (HBTT) Testing results for 627 negative control sera

| | Hybrid ELISA | Hybrid ELISA | |
|---|---|---|---|
| | Positive | Negative | Total |
| HB TT Positive | 7 | 0 | 7 |
| HB TT Negative | 2 | 618 | 620 |
| Total | 9 | 618 | 627 |

| | |
|---|---|
| McNemar's two-tailed p-value | 0.5 |

### Analytical Sensitivity and Hook Effect

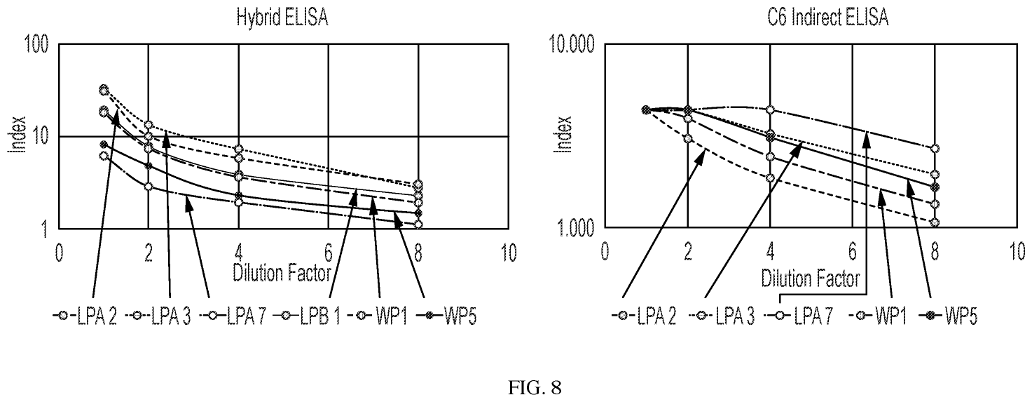

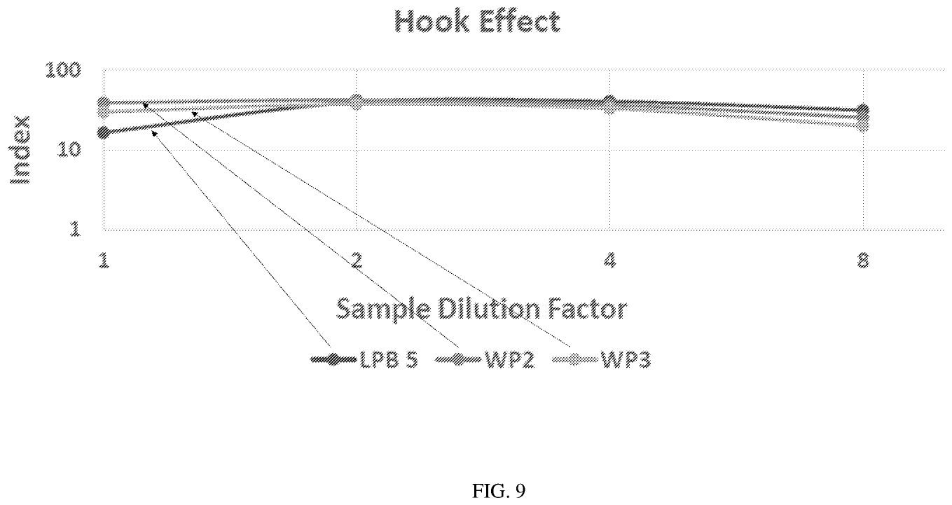

Testing serial dilutions of several Lyme sera in the Hybrid ELISA vs. a conventional C6 peptide indirect ELISA showed a slightly greater effect of dilution in the Hybrid ELISA (FIG. 8). This is not likely to affect performance of the Hybrid ELISA in practice, as it relies on an undiluted (neat) serum sample. Testing of several highly positive Lyme sera at increasing dilutions revealed a modest hook effect for one serum sample, which did not affect the final result, which remained strongly positive (FIG. 9).

### Stability and Reproducibility

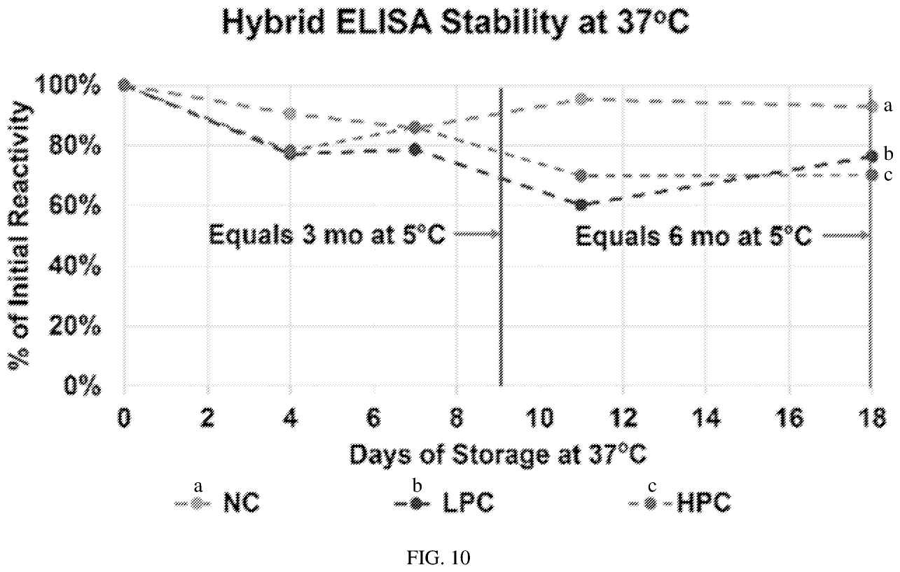

An accelerated stability study was carried out by storing the Hybrid ELISA reagents and coated microplate at various temperatures and measuring assay performance vs. initial levels at intervals of several days. Results showed that after an initial drop in reactivity in the first few days associated with drying the microplates, average reactivity remained at about 98% of day 4 levels after 18 days at 37° C., which translates in the Arrhenius model (Magari et al., J Clin Lab Anal. 2002; 16 (5): 221-6; Accelerated Aging Calculator, 2022) to roughly 6 months at 4° C. (FIG. 10). Accordingly, the Hybrid ELISA reagents appear to be stable and thus far a target shelf life of two years at 4° C. appears feasible.

The results presented this Example demonstrate the feasibility of the Hybrid Lyme ELISA as a one-tier test for Lyme disease, having shown that it outperforms the Standard Two Tier Testing protocol significantly in sensitivity while providing equivalent specificity. The potential to increase sensitivity without decreasing specificity has been a significant challenge for Lyme disease testing, and the sole approach to date that has shown some success has been to utilize two ELISAs in series. The Hybrid ELISA, based on a novel immunochemical concept, offers a new solution with the potential to reduce the two-tier procedure to a single tier procedure.

### Example 4. Manufacture of Hybrid ELISA Kits for Clinical Use

Three pilot lots of approximately 300 kits each are manufactured. Microplates are coated using the high-throughput Thermo Matrix automated microplate dispenser/coater (capacity ≥250 microplates/hour), and conjugate and other solutions are prepared in bulk and dispensed into bottles or vials using the Hamilton Microlab 600 semi-automated dispenser. Kits are subject to Quality Control testing. A Quality Control (QC) serum panel comprising 6-10 sera covering a range from negative controls to low, moderate and high reactivity will be developed, along with corresponding assay protocols, for use in qualifying reagents that are produced for inclusion in manufactured kits, and for final QC approval of kit lots.

### Example 5. Clinical and Analytical Evaluation of the Hybrid ELISA

The clinical study design is modeled after that used by Zeus Scientific related to its K190907 and K191240 ELISA kits for use in a modified two-tier testing (MTTT) protocol, in which the second ELISA replaces the traditional Western Blot and is also consistent with FDA's previous guidance for determining performance characteristics of Lyme diagnostic devices (FDA, Vol. 78, Federal Register. 2013). The study comprises a retrospective study of ≥100 well-characterized Lyme serum samples from patients (1) in the early acute stage presenting with erythema migrans or culture-positive, between 0-3 months after disease onset (2) in the convalescent stage, with erythema migrans or culture-positive results, between 3-12 months after disease onset; and (3) with manifestations of Lyme disease other than erythema migrans, including Lyme Arthritis, Neuroborreliosis and Lyme Carditis. Convalescent patients are treated with antibiotics upon the initial diagnosis, which may curtail the immune response to the spirochetal infection, and thus provides a sample subset in which any such effect is observed. Patients with later stage symptoms are optionally divided between those treated vs. not treated prior to the date of sample acquisition, for the same reason. The study also comprises a prospective study in which undiagnosed patients in the target population for the test from 3 different geographical regions are tested. An analytical specificity study is also carried out in which ≥100 sera from healthy controls in both endemic and non-endemic areas are tested. For all of the above studies, samples are tested on the investigational device and in parallel on FDA-approved ELISA and Western Blot assays as the predicate device at 3 sites, which optionally include the manufacturer's laboratory as one site. Finally, the investigational device is evaluated for cross-reactivity with a variety of serum samples from patients with other disease conditions (e.g., tick-borne relapsing fever, syphilis, rickettsial diseases, ehrlichiosis, babesiosis, leptospirosis, parvovirus B19, influenza viruses, Epstein-Barr virus, cytomegalovirus, H. pylori); and from patients with conditions with symptoms that overlap late Lyme disease (e.g., chronic fatigue syndrome, fibromyalgia, rheumatoid arthritis, autoimmune diseases, and multiple sclerosis), for interference (hemoglobin, bilirubin, etc.), and for reproducibility between sites, operators, assay runs, and assay lots. FDA-approved Trinity Captiva IgG/IgM ELISA, which uses whole-cell sonicate as antigen, and the FDA-approved Gold Standard Diagnostics recombinant IgG and IgM immunoblot kits are used as predicate devices. External sites use the FDA-approved ELISA and immunoblot kits that are in routine use for Lyme testing at their respective laboratories as predicate devices. The conventional STTT protocol is used throughout as the predicate for this study.

### External Sites

Three geographically distinct external sites participate in the clinical evaluation, optionally regional healthcare networks with multiple local clinics at which patients with tick-borne illnesses are seen and/or located in areas known to be highly endemic for Lyme disease The sites carry out testing of retrospective and prospective samples with the Hybrid ELISA in parallel with testing using standardized two-tier ELISA and Western Blot kits.

### Example 6. Retrospective Evaluation of Sensitivity and Specificity on Well-Characterized Lyme Sera, Healthy Control Sera and Sera from Patients with Other Disease Conditions

A panel of ˜500 serum samples corresponding to the categories defined by FDA as delineated in Example 6 are assembled and roughly equal portions distributed for testing at least 2 sites. The panel comprises approximately 200 Lyme serum samples, 100 sera from patients with other disease conditions, 100 sera from healthy endemic and 100 sera from healthy non-endemic controls. Sample size justification: The Zeus Scientific study described in FDA 510 (k) approval K19097 included a retrospective panel of 356 serum specimens, comprising 166 sera from Lyme patients, 90 from patients with other disease conditions, and 100 healthy controls. The Lyme sera portion of the panel is divided roughly equally between sera from early acute cases presenting with EM, convalescent cases that presented with EM, and cases with extracutaneous manifestations of disseminated infection (Lyme Arthritis, Neuroborreliosis, Lyme Carditis). Serum samples for this purpose from diagnosed Lyme patients with clinical histories and available serological testing results are obtained from testing sites, CDC and the Lyme Disease Biobank (LDB). The CDC has two serum panels-Research Panel II comprising 92 samples and a pre-marketing panel comprising 290 samples, intended to support 510 (k) approval (Molins et al., J Clin Microbiol. 2014 Oct. 1; 52 (10): 3755-62). The Lyme Disease Biobank has a collection of 298 serum samples from Lyme patients and 252 controls. Additional serum samples from healthy controls and from patients with other disease conditions are obtained from the same sources and from commercial vendors (Boca Biolistics, SeraCare).

### Analysis

The sensitivity of the Hybrid ELISA in detecting confirmed Lyme positive samples in the Retrospective study is compared with that of standard two-tier testing based on archival results from the sample sources. Samples from patients presenting with EM or with culture-positive or PCR-positive blood or skin biopsy results are considered true positives irrespective of their two-tier testing results. Specificity of the Hybrid ELISA is compared with STTT on healthy controls and patients with other, potentially cross-reactive disease conditions; for these sample sets, true negative samples are defined by a negative STTT result, while samples with positive STTT results are considered true positives. Sensitivity and specificity of the Hybrid ELISA is compared vs. true status and vs. STTT using McNemar's test to determine the significance of differences between assay results.

### Results

Based on similar studies carried out previously as reported in Table 5 and Tables 7-10, the sensitivity of the Hybrid ELISA in the Retrospective study is expected to be approximately 95-100% vs. STTT for later stage Lyme sera, including Lyme arthritis and Neuroborreliosis sera. The sensitivity of Hybrid ELISA for early stage Lyme sera is expected to be at least 30% greater than that of STTT, yielding a statistically significant improvement, although the exact sensitivity will depend on the timing of the sample draw following disease onset. Negligible cross-reactivity is expected in Hybrid ELISA with sera from patients with other disease conditions, and specificity above 99.5% vs. STTT for sera from healthy controls, or statistically equivalent.

### Example 7. Evaluation of Inter-Assay, Inter-Run, Inter-Lot and Inter-Operator Reproducibility and Stability of Hybrid ELISA

A reproducibility serum panel comprising 6-8 sera with levels of reactivity in the Hybrid ELISA ranging from non-reactive to weakly positive to strongly positive are generated from sera collections and tested over multiple days, operators, assay runs and kit lots according to standard procedures for this purpose as described in CLSI documents EP5-A2 (CLSI, 2004), EP12-A (CLSI, 2008), and EP15-A2 (CLSI, 2006).

Kits are tested for stability using the same serum panel in an accelerated protocol, where 26 weeks storage at 25° C. is predicted to equal 2 years at 5° C. (Magari et al., J Clin Lab Anal. 2002; 16 (5): 221-6; Accelerated Aging Calculator, 2022). Kits will be considered stable for the period during which no change in interpretation of any serum panel member vs. the initial interpretation is registered.

### Results

It is expected that the Hybrid ELISA kit will pass the above stability criteria in the accelerated protocol, predicting acceptable stability over a wo-year period when stored at 5° C.

### Example 8. Hybrid Lyme Lateral Flow Immunoassay

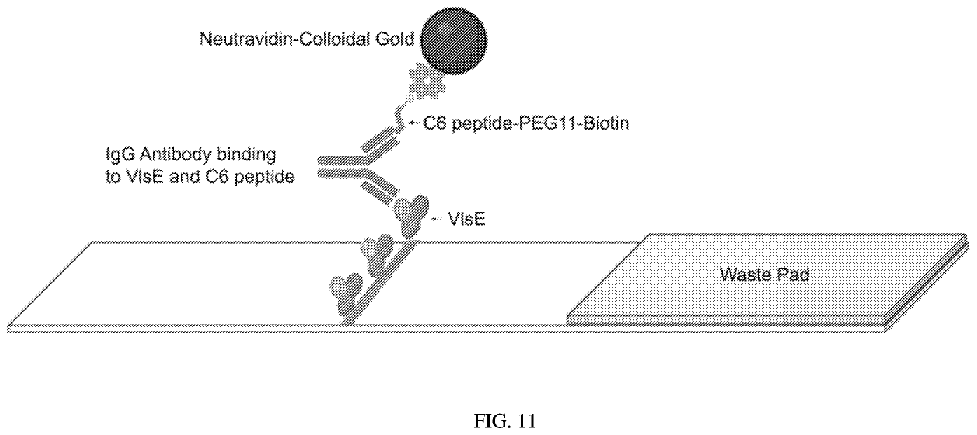

A point-of-care Hybrid Lateral Flow Immunoassay test to identify Lyme disease antibodies in subject blood serum using the two antigen approach and Hybrid Immunoassay technology described herein for the Hybrid ELISA Immunoassay was developed. A schematic of the Hybrid Lyme Lateral Flow Immunoassay design is shown in FIG. 11. In FIG. 11, the pink test line contains VlsE antigen. Blood serum from a subject bound to the biotinylated C6 peptide binds to VlsE due to the bivalent nature of the antibody, and the immunocomplexes are detected by NeutrAvidin Protein conjugated to colloidal gold.

A first antigen (VlsE recombinant protein) was striped (immobilized) on the nitrocellulose membrane at concentrations between 1-3 mg/ml, with 3 mg/ml found to be optimal, to form the test line. A second antigen (a biotinylated C6 peptide) was titrated in the range 0.003-0.3 μg/mL, and 0.01 μg/mL was selected as the optimal concentration. C6-PEG11-Biotin was diluted in running solution (100 mM Tris, 1% Tween20), then mixed with 10 μL of serum sample and applied to the nitrocellulose membrane. Neutravidin-labeled colloidal gold (40 nm) was used to detect the VlsE-Antibody-C6-PEG11-Biotin complex via binding to the biotin.

The assay protocol was as follows: First, the strip comprising nitrocellulose membrane with striped test line and waste pad was dipped into the mixture of C6-Biotin and the subject blood serum sample. As the solution moved through the membrane, complexes of human antibody bound to C6-biotin were bound to VlsE on the test line. After 5 minutes, the strip was dipped in wash solution for 15 seconds, then dipped in a solution containing Colloidal Gold-Neutravidin at an optical density of 1.0 OD. After incubation for 10 min, the results were analyzed visually and by a lateral flow strip reader. The presence of antibody to Lyme antigens was indicated by the appearance of a pink line at the position of the test line. The total assay time was 15 minutes.

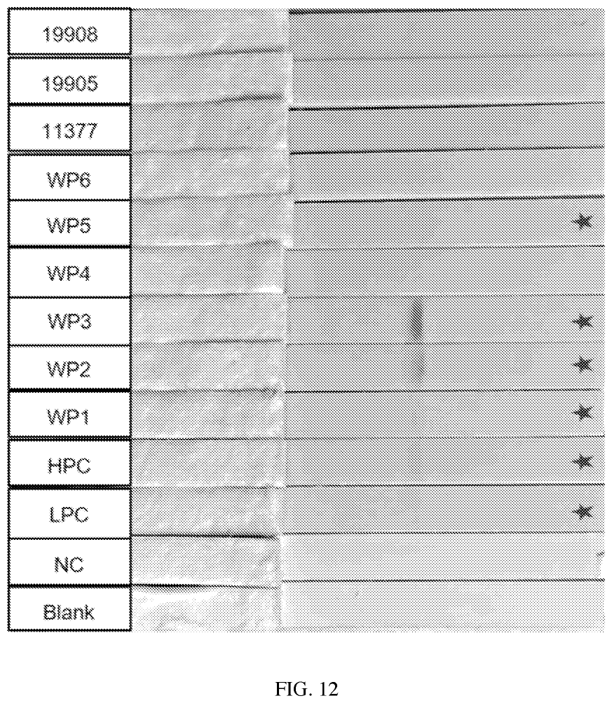

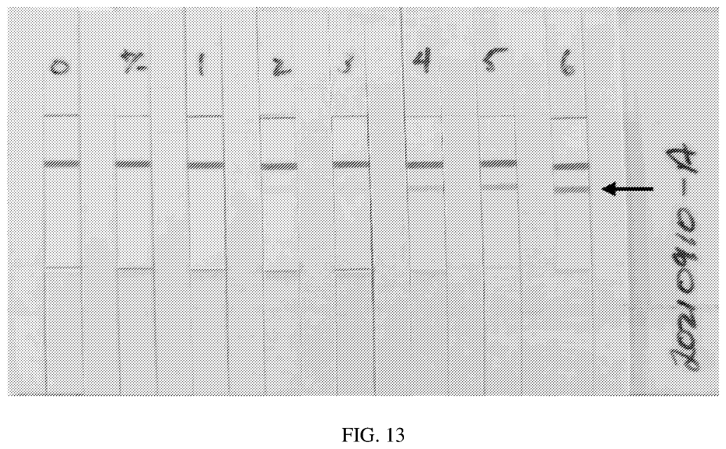

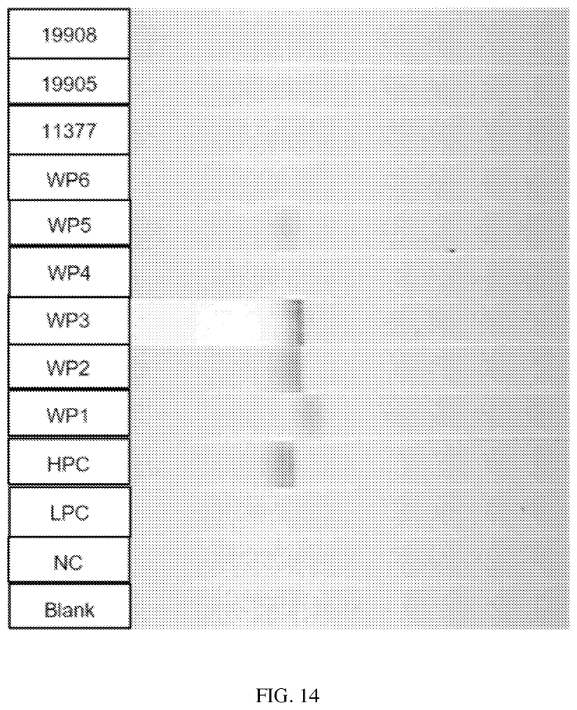

The Hybrid Lyme Lateral Flow Immunoassay was evaluated on a panel comprising 6 serum samples from Lyme disease-positive subjects (as previously characterized by the Hybrid ELISA Immunoassay), 3 sera from healthy blood donors, and 3 assay controls. Four out of 6 Lyme disease-positive sera were detected visually on the Hybrid Lateral Flow Immunoassay (FIG. 12; stars indicate positive results, sample identifiers appear at the top of FIG. 12), scored according to a visual Read Guide, in which band intensity is correlated with a score value (FIG. 13). The same sera were detected by an electronic reading device (Axxin AX-2X-S Lateral Flow Reader, FIG. 14; sample identifiers appear at the top of FIG. 14). The samples that were detected on the Hybrid Lateral Flow Immunoassay yielded Lyme index values (equal to signal/cut-off, or ELISA absorbance of sample divided by assay cut-off value) in the Hybrid ELISA Immunoassay ranging from 21.33 to 47.62 (Table 14).

Table 14 shows results of the Hybrid Lateral Flow Immunoassay compared to results of a Hybird ELISA Immunoassay from Examples 1-7. Visual read was scored using the Read Guide in FIG. 13. Axxin read was calculated as intensity of the band measured at the test line divided by cut-off, with cut-off equal to the average intensity of the negative controls plus two standard deviations. Hybrid ELISA Immunoassay Index is calculated as ELISA absorbance of a sample divided by the cut-off.

TABLE 14
Hybrid Lateral Flow Immunoassay
vs. Hybrid ELISA Immunoassay

| | | Hybrid | Hybrid | Hybrid |
|---|---|---|---|---|
| | | Lateral Flow | Lateral Flow | ELISA Lyme |
| | Sample ID | Visual Read | Axxin Read | Index |
| Assay | Negative | 0 | 0.66 | 0.70 |
| Controls | Control | | | |
| | Low Positive | 1 | 1.57 | 3.05 |
| | Control | | | |
| | High Positive | 2 | 16.02 | 31.27 |
| | Control | | | |
| Lyme | WP1 | 2 | 8.81 | 27.13 |
| Positive | WP2 | 3 | 17.11 | 47.62 |
| Samples | WP3 | 5 | 26.03 | 47.62 |
| | WP4 | 0 | 0.87 | 1.03 |
| | WP5 | 1 | 6.04 | 21.33 |
| | WP6 | 0 | 0.55 | 1.59 |
| Healthy | 11377 | 0 | 0.80 | NT |
| Blood | 19906 | 0 | 0.55 | NT |
| Donor | 19908 | 0 | 0.80 | NT |
| Samples | | | | |

The two Lyme sera that were not detected by the Hybrid Lateral Flow Immunoassay had Hybrid ELISA Immunoassay index values of 1.03 and 1.59, both close to the cut-off. The Axxin read values were qualitatively parallel to the Hybrid ELISA Immunoassay values, differing only for several samples at the low end of the range. These data suggest that the Hybrid Lateral Flow Immunoassay results correspond very closely to the Hybrid ELISA Immunoassay results obtained for the same serum samples.

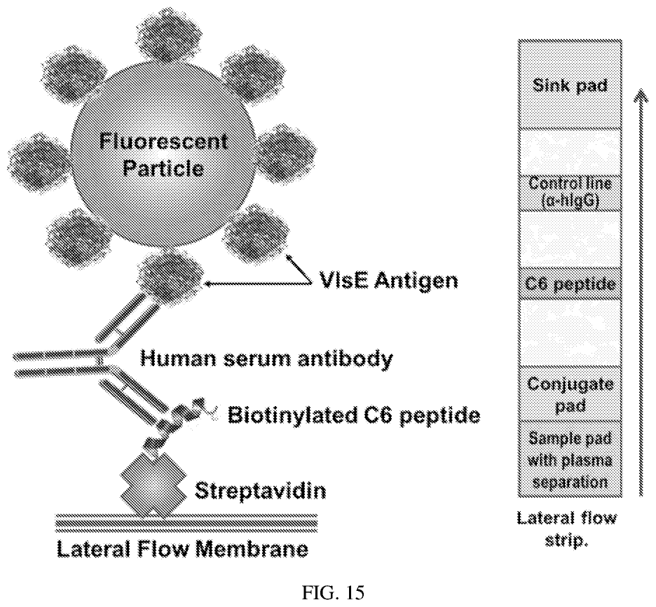

In a second experiment, a Hybrid Lateral Flow Immunoassay was developed with C6 peptide-streptavidin conjugate striped on the test line, and fluorescent Europium particles coated with VlsE applied as the soluble antigen conjugate in the conjugate pad (FIG. 15). In this version, all reagents were dried onto the strip. The test was evaluated on a panel of 20 serum samples from early stage Lyme disease-positive subjects, including paired acute and convalescent samples collected from the same individuals. Results were read using a Qiagen ESEQuant fluorescent lateral flow reader, which enabled quantification of fluorescence intensity on the strip. An index value was calculated as fluorescence intensity divided by a cut-off value, and samples with index values greater than 1 were interpreted as positive in the assay (Table 15).

Table 15 shows the Hybrid Lateral Flow Immunoassay evaluated on a Lyme serum panel comprising acutes (indicated as “A”) and convalescent (indicated as “C”) sera from early-stage Lyme disease-positive subjects. The Hybrid Lateral Flow Immunoassay used a biotinylated C6 peptide conjugated to streptavidin striped on the test line on the nitrocellulose strip at 3 mg/mL and VlsE conjugated to fluorescent Europium in place of colloidal gold. The sample volume was 10 μL of undiluted serum. Fluorescence was read and quantified using a Qiagen ESEQuant reader. Lateral flow results were scored as positive when the index (signal/cut-off value) was greater than 1. The Hybrid Lateral Flow Immunoassay are compared against three Lyme ELISA immunoassays, as well as IgG and IgM immunoblot results.

TABLE 15
Hybrid Lateral Flow Immunoassay Evaluated on Lyme Serum Panel

| | Hybrid | Hybrid | | | | | |
|---|---|---|---|---|---|---|---|
| | Lateral | Lateral | Wampole | | DiaSorin | | |
| Sample | Flow Peak | Flow | WCS | Immunetics | Liaison | IgG | IgM |
| # | Height | Index | ELISA | C6 ELISA | VISE | Blot | Blot |
| 1A | 1033.2 | 23.48 | Positive | Positive | Positive | Negative | Positive |
| 1C | 2484.2 | 56.47 | Positive | Positive | Positive | Negative | Positive |
| 2A | 26.5 | 0.60 | Negative | Negative | Negative | Negative | Negative |
| 2C | 734.9 | 16.70 | Positive | Equivalent | Negative | Negative | Positive |
| 3A | 12.4 | 0.28 | Positive | Negative | Negative | Negative | Negative |
| 3C | 10.6 | 0.24 | Positive | Negative | Negative | Negative | Negative |
| 4A | 1261.9 | 28.68 | Negative | Positive | Positive | Negative | Positive |
| 4C | 326.4 | 7.42 | Positive | Positive | Positive | Negative | Positive |
| 5A | 7.7 | 0.17 | Positive | Negative | Negative | Negative | Negative |
| 6A | 4606.3 | 104.70 | Positive | Positive | Positive | Negative | Positive |
| 7A | 240.7 | 5.47 | Positive | Equivalent | Negative | Negative | Negative |
| 7C | 550.5 | 12.51 | Positive | Equivalent | Negative | Negative | Negative |
| 8A | 156.8 | 3.56 | Positive | Positive | Positive | Negative | Negative |
| 8C | 149.3 | 3.39 | Positive | Positive | ND | Negative | Negative |
| 9A | 6.4 | 0.14 | Negative | Negative | Negative | Negative | Negative |
| 10A | 6.5 | 0.15 | Negative | Negative | Negative | Negative | Negative |
| 11A | 425.0 | 9.66 | Positive | Positive | Positive | Negative | Negative |
| 11C | 405.8 | 9.22 | Positive | Positive | ND | Negative | Negative |
| 12A | 1071.9 | 24.36 | Negative | Positive | Positive | Negative | Negative |
| 12C | 1000.2 | 22.73 | Positive | Positive | ND | Negative | Positive |
| % | 70% | | 75% | 70% | 59% | 0% | 35% |
| Detected | (14/20) | | (15/20) | (14/20) | (10/17) | (0/20) | (7/20) |

The Hybrid Lateral Flow Immunoassay detected 14 out of 20, or 70% of the serum samples as positive. By comparison, 3 FDA-cleared Lyme ELISAs using whole cell sonicate antigen, C6 peptide, and VlsE, respectively, detected 15/20 (75%), 14/20 (70%), and 10/17 (59%) of the same serum samples. An IgM immunoblot detected 7/20 (35%) of the sera, while none were detected by an IgG immunoblot. Accordingly, the Hybrid Lateral Flow Immunoassay in this fluorescence-based version, with C6 peptide immobilized on the test line and VlsE in solution, demonstrated sensitivity similar to that of conventional FDA-approved Lyme ELISA assays, and better than that of an IgM immunoblot.

### INCORPORATION BY REFERENCE

The present application refers to various issued patent, published patent applications, scientific journal articles, and other publications, all of which are incorporated herein by reference. The details of one or more embodiments of the present disclosure are set forth herein. Other features, objects, and advantages of the present disclosure will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

### REFERENCES

1. Aucott J, Morrison C, Munoz B, Rowe P C, Schwarzwalder A, West S K. Diagnostic challenges of early Lyme disease: Lessons from a community case series. BMC Infect Dis. 2009 December; 9 (1): 79.
2. Aucott J N, Crowder L A, Yedlin V, Kortte K B. Bull's-Eye and Nontarget Skin Lesions of Lyme Disease: An Internet Survey of Identification of Erythema Migrans. Dermatol Res Pract. 2012 October; 2012:1-6.
3. Moore A, Nelson C, Molins C, Mead P, Schriefer M. Current Guidelines, Common Clinical Pitfalls, and Future Directions for Laboratory Diagnosis of Lyme Disease, United States. Emerg Infect Dis. 2016 July; 22 (7): 1169-77.
4. Centers for Disease Control and Prevention (CDC). Recommendations for Test Performance and Interpretation From the Second National Conference on Serologic Diagnosis of Lyme Disease. MMWR Morb Mortal Wkly Rep. 1995; 44 (31): 590-1.
5. Branda J A, Body B A, Boyle J, Branson B M, Dattwyler R J, Fikrig E, et al. Advances in Serodiagnostic Testing for Lyme Disease Are at Hand. Clin Infect Dis. 2018; 66 (7): 1133-9.
6. Theel E S. The past, present, and (possible) future of serologic testing for lyme disease. J Clin Microbiol [Internet]. 2016; 54 (5): 1191-6. Available from: www.ncbi.nlm.nih.gov/pmc/articles/PMC4844714/pdf/zjm1191.pdf
7. Bacon R M, Kugeler K J, Mead P S. Surveillance for Lyme Disease—United States, 1992-2006. Morb Mortal Wkly Rep. 2008; 57 ((SS10)): 1-9.
8. Beard, C. B., R. J. Eisen, C. M. Barker, J. F. Garofalo, M. Hahn, M. Hayden, A. J. Monaghan, N. H. Ogden and PJS. Vectorborne Diseases. The Impacts of Climate Change on Human Health in the United States: A Scientific Assessment. U.S. Global Change Research Program, Washington, D C. 2016. p. 129-156.
9. Nelson C A, Saha S, Kugeler K J, Delorey M J, Shankar M B, Hinckley A F, et al. Incidence of clinician-diagnosed lyme disease, United States, 2005-2010. Emerg Infect Dis. 2015;
10. Hinckley A F, Connally N P, Meek J I, Johnson B J, Kemperman M M, Feldman K A, et al. Lyme disease testing by large commercial laboratories in the United States. Clin Infect Dis. 2014; 59 (5): 676-81.
11. Kugeler K J, Schwartz A M, Delorey M J, Mead P S, Hinckley A F. Estimating the frequency of lyme disease diagnoses, United States, 2010-2018. Emerg Infect Dis. 2021; 27 (2): 616-9.
12. Adrion E R, Aucott J, Lemke K W, Weiner J P. Health Care Costs, Utilization and Patterns of Care following Lyme Disease. Brissette CA, editor. PLOS One. 2015 February; 10 (2): e0116767.
13. Berry K, Bayham J, Meyer S R, Fenichel E P. The Allocation of Time and Risk of Lyme: A Case of Ecosystem Service Income and Substitution Effects. Environ Resour Econ. 2017 April; 1-20.
14. Afzelius A. No Title. Arch Dermatol Syph. 1910; 101:403-6.
15. Wormser G P, Dattwyler R J, Shapiro E D, Halperin J J, Steere A C, Klempner M S, et al. The Clinical Assessment, Treatment, and Prevention of Lyme Disease, Human Granulocytic Anaplasmosis, and Babesiosis: Clinical Practice Guidelines by the Infectious Diseases Society of America. 2006; 43.
16. Borgermans L, Goderis G, Vandevoorde J, Devroey D. Relevance of chronic lyme disease to family medicine as a complex multidimensional chronic disease construct: a systematic review. Int J Family Med. 2014; 2014:138016.
17. Centers for Disease Control and Prevention. Notice to Readers: Recommendations for Test Performance and Interpretation from the Second National Conference on Serologic Diagnosis of Lyme Disease. MMWR. 1995; 44 (31): 590-1.
18. Nigrovic L E, Bennett J E, Balamuth F, Levas M N, Chenard R L, Maulden A B, et al. Accuracy of Clinician Suspicion of Lyme Disease in the Emergency Department. Pediatrics. 2017 December; 140 (6): e20171975.
19. Waddell L A, Greig J, Mascarenhas M, Harding S, Lindsay R, Ogden N. The Accuracy of Diagnostic Tests for Lyme Disease in Humans, A Systematic Review and Meta-Analysis of North American Research. Stevenson B, editor. PLOS One [Internet]. 2016 Dec. 21 [cited 2017 Dec. 20]; 11 (12): e0168613. Available from: http://dx.plos.org/10.1371/journal.pone.0168613
20. Branda J A, Strle K, Nigrovic L E, Lantos P M, Lepore T J, Damle N S, et al. Evaluation of modified 2-tiered serodiagnostic testing algorithms for early Lyme disease. Clin Infect Dis. 2017; 64 (8): 1074-80.
21. Branda J A, Linskey K, Kim Y A, Steere A C, Ferraro M J. Two-tiered antibody testing for lyme disease with use of 2 enzyme immunoassays, a whole-cell sonicate enzyme immunoassay followed by a vlse c6 peptide enzyme immunoassay. Clin Infect Dis. 2011;
22. Wormser G P, Schriefer M, Aguero-Rosenfeld M E, Levin A, Steere A C, Nadelman R B, et al. Single-tier testing with the C6 peptide ELISA kit compared with two-tier testing for Lyme disease. Diagn Microbiol Infect Dis [Internet]. 2013 January [cited 2018 Jun. 16]; 75 (1): 9-15. Available from: http://www.ncbi.nlm.nih.gov/pubmed/23062467
23. Pegalajar-Jurado A, Schriefer M E, Welch R J, Couturier M R, Mackenzie T, Clark R J, et al. Evaluation of Modified Two-Tiered Testing Algorithms for Lyme Disease Laboratory Diagnosis Using Well-Characterized Serum Samples. J Clin Microbiol [Internet]. 2018 Aug. 1 [cited 2018 Aug. 31]; 56 (8): e01943-17. Available from: http://www.ncbi.nlm.nih.gov/pubmed/29743307
24. Branda J A, Body B A, Boyle J, Branson B M, Dattwyler R J, Fikrig E, et al. Advances in Serodiagnostic Testing for Lyme Disease Are at Hand. Clin Infect Dis. 2018 December; 66 (7): 1133-9.
25. Clinical Laboratory News. FDA Clears Lyme Disease Tests that Upend Two-Tier Approach [Internet]. AACC. 2019. Available from: www.aacc.org/cln/articles/2019/october/fda-clears-lyme-disease-tests-that-upend-two-tier-approach
26. Food and Drug Administration-FDA. K190907 510 (k) SUBSTANTIAL EQUIVALENCE DETERMINATION DECISION SUMMARY [Internet]. 2019. Available from: www.accessdata.fda.gov/cdrh_docs/reviews/K190907.pdf
27. Ting Liang F, Steere A C, Marques A R, B Johnson B J, Miller J N, Philipp M T. Sensitive and Specific Serodiagnosis of Lyme Disease by Enzyme-Linked Immunosorbent Assay with a Peptide Based on an Immunodominant Conserved Region of Borrelia burgdorferi VlsE. J Clin Microbiol. 1999; 37 (12): 3990-6.
28. Liang F T, Aberer E, Cinco M, Gern L, Hu C M, Lobet Y N, et al. Antigenic conservation of an immunodominant invariable region of the VlsE lipoprotein among European pathogenic genospecies of Borrelia burgdorferi SL. J Infect Dis. 2000; 182 (5): 1455-62.
29. Bacon R M, Biggerstaff B J, Schriefer M E, Gilmore R D, Philipp M T, Steere A C, et al. Serodiagnosis of Lyme disease by kinetic enzyme-linked immunosorbent assay using recombinant VlsE1 or peptide antigens of Borrelia burgdorferi compared with 2-tiered testing using whole-cell lysates. J Infect Dis. 2003; 187 (8): 1187-99.
30. Wormser G P, Liveris D, Hanincová K, Brisson D, Ludin S, Stracuzzi V J, et al. Effect of Borrelia burgdorferi genotype on the sensitivity of C6 and 2-tier testing in North American patients with culture-confirmed Lyme disease. Clin Infect Dis. 2008 October; 47 (7): 910-4.
31. Wormser G P, Nowakowski J, Nadelman R B, Visintainer P, Levin A, Aguero-Rosenfeld M E. Impact of clinical variables on Borrelia burgdorferi-specific antibody seropositivity in acute-phase sera from patients in North America with culture-confirmed early Lyme disease. Clin Vaccine Immunol. 2008; 15 (10): 1519-22.
32. Branda J A, Strle F, Strle K, Sikand N, Ferraro M J, Steere A C. Performance of United States serologic assays in the diagnosis of lyme borreliosis acquired in Europe. Clin Infect Dis. 2013;
33. Wormser G P, Tang A T, Schimmoeller N R, Bittker S, Cooper D, Visintainer P, et al. Utility of serodiagnostics designed for use in the United States for detection of Lyme borreliosis acquired in Europe and vice versa. Med Microbiol Immunol. 2014;
34. Lipsett S C, Branda J A, McAdam A J, Vernacchio L, Gordon C D, Gordon C R, et al. Evaluation of the C6 Lyme Enzyme Immunoassay for the Diagnosis of Lyme Disease in Children and Adolescents. Clin Infect Dis. 2016; 63 (1 October): 922-8.
35. Zhang J R, Hardham J M, Barbour a G, Norris S J. Antigenic variation in Lyme disease borreliae by promiscuous recombination of VMP-like sequence cassettes. Cell. 1997; 89 (2): 275-85.
36. LAWRENZ M, MAGNARELLI L A, FIKRIG E, NORRIS S J. Comparative reactivity of human sera to recombinant VlsE and other Borrelia burgdorferi antigens in class-specific enzyme-linked immunosorbent assays for Lyme borreliosis. J Med Microbiol. 2002 August; 51 (8): 649-55.
37. Lawrenz M B, Hardham J M, Owens R T, Nowakowski J, Steere A C, Wormser G P, et al. Human antibody responses to VlsE antigenic variation protein of Borrelia burgdorferi. J Clin Microbiol. 1999 December; 37 (12): 3997-4004.
38. Pritt B S, Mead P S, Johnson D K H, Neitzel D F, Respicio-Kingry L B, Davis J P, et al. Identification of a novel pathogenic Borrelia species causing Lyme borreliosis with unusually high spirochaetaemia: a descriptive study. Lancet Infect Dis. 2016 May; 16 (5): 556-64.
39. Lantos P M, Branda J A, Boggan J C, Chudgar S M, Wilson E A, Ruffin F, et al. Poor Positive Predictive Value of Lyme Disease Serologic Testing in an Area of Low Disease Incidence. Clin Infect Dis. 2015 November; 61 (9): 1374-80.
40. Gregson D, Evans G, Patrick D, Bowie W. Lyme disease: How reliable are serologic results? CMAJ. 2015 November; 187 (16): 1193-4.
41. Fallon B A, Pavlicova M, Coffino S W, Brenner C. A Comparison of Lyme Disease Serologic Test Results From 4 Laboratories in Patients With Persistent Symptoms After Antibiotic Treatment. Clin Infect Dis. 2014 December; 59 (12): 1705-10.
42. System and methods for detection of Bacillus anthracis related analytes in biological fluids. 2004 [cited 2017 Dec. 24]; Available from: www.google.com/patents/U.S. Pat. No. 7,262,019
43. Biagini R E, Sammons D L, Smith J P, Mackenzie B A, Striley C A F, Snawder J E, et al. Rapid, sensitive, and specific lateral-flow immunochromatographic device to measure anti-anthrax protective antigen immunoglobulin g in serum and whole blood. Clin Vaccine Immunol. 2006 May; 13 (5): 541-6.
44. Alere. Alere Determine™ HIV-1/2 Ag/Ab Combo Package Insert.
45. Embers M E, Jacobs M B, Johnson B J B, Philipp M T. Dominant epitopes of the C6 diagnostic peptide of Borrelia burgdorferi are largely inaccessible to antibody on the parent VlsE molecule. Clin Vaccine Immunol. 2007;
46. Target product profile for a test for recent HIV infection. 2017;
47. Thomas M C, Fernández-Villegas A, Carrilero B, Marañón C, Saura D, Noya O, et al. Characterization of an immunodominant antigenic epitope from Trypanosoma cruzi as a biomarker of chronic Chagas' disease pathology. Clin Vaccine Immunol. 2012; 19 (2): 167-73.
48. Fumagalli M J, Figueiredo L T M, Aquino V H. Linear and Continuous Flavivirus Epitopes From Naturally Infected Humans. Front Cell Infect Microbiol. 2021; 11 (August): 1-16.
49. Shan G. Improved confidence intervals for the Youden Index. PLOS One. 2015; 10 (7): 1-19.
50. Marcus D. Ruopp, Perkins N J, Whitcomb B W, Schisterman E F. Youden Index and Optimal Cut-Point Estimated from Observations Affected by a Lower Limit of Detection. Biom J. 2008; 23 (3): 419-30.
51. Baratloo A, Safari S, Elfil M, Negida A. Evidence Based Emergency Medicine Part 3: Positive and Negative Likelihood Ratios of Diagnostic Tests. Emerg (Tehran, Iran) [Internet]. 2015; 3 (4): 170-1. Available from: http://www.ncbi.nlm.nih.gov/pubmed/26495411% 0Ahttp://www.pubmedcentral.nih.gov/artic lerender.fcgi?artid=PMC4608341
52. Chicco D, Jurman G. The advantages of the Matthews correlation coefficient (MCC) over F1 score and accuracy in binary classification evaluation. BMC Genomics. 2020; 21 (1): 1-13.
53. Chicco D, Tötsch N, Jurman G. The matthews correlation coefficient (Mcc) is more reliable than balanced accuracy, bookmaker informedness, and markedness in two-class confusion matrix evaluation. BioData Min. 2021; 14:1-22.
54. Magari R T, Murphy K P, Fernandez T. Accelerated stability model for predicting shelf-life. J Clin Lab Anal. 2002; 16 (5): 221-6.
55. Accelerated Aging Calculator, Medical Devices, Package Testing, AAT Calculator [Internet]. [cited 2022 Jan. 1]. Available from: www.rmtesting.com/accelerated-aging-calculator-medical-device-packaging-testing.php #calc
56. Food and Drug Administration-FDA. Establishing the Performance Characteristics of In Vitro Diagnostic Devices for the Detection of Antibodies to Borrelia burgdorferi; Guidance for Industry and Food and Drug Administration Staff; Availability. [Internet]. Vol. 78, Federal Register. 2013. Available from: www.fda.gov/media/85572/download
57. Molins C R, Sexton C, Young J W, Ashton L V., Pappert R, Beard C B, et al. Collection and Characterization of Samples for Establishment of a Serum Repository for Lyme Disease Diagnostic Test Development and Evaluation. J Clin Microbiol [Internet]. 2014 Oct. 1 [cited 2018 Jun. 16]; 52 (10): 3755-62. Available from: http://jcm.asm.org/cgi/doi/10.1128/JCM.01409-14
58. Clinical and Laboratory Standards Institute. Evaluation of Precision Performance of Quantitative Measurement Methods; Approved Guideline. EP5-A2. Clinical and Laboratory Standards Institute, Wayne PA.; 2004.
59. Institute. C and L S. User Protocol for Evaluation of Qualitative Test Performance; Approved Guideline. EP12-A2. Clinical and Laboratory Standards Institute, Wayne PA.; 2008.
60. Clinical and Laboratory Standards Institute. User Verification of Performance for Precision and Trueness; Approved Guideline. EP15-A2. Clinical and Laboratory Standards Institute, Wayne PA.; 2006.
61. Jennings L, Van Deerlin V M, Gulley M L. Recommended principles and practices for validating clinical molecular pathology tests. Arch Pathol Lab Med. 2009; 133 (5): 743-55.
62. William Castellani M, College of American Pathologists. Laboratory Developed Tests. 2015; 38. Available from: http://www.fda.gov/MedicalDevices/ProductsandMedicalProcedures/In VitroDiagnostics/ucm407296.htm (retrieved Feb. 9, 2016)
63. Centers for Medicare & Medicaid Services. Verification of Performance Specifications. 1988.
64. CLIA. Compliant Analytical Method Validation Plan and Template FOR LRN-C LABORATORIES. Public Heal Lab [Internet]. 2013; (December): 1-14. Available from: http://www.aphl.org/aphlprograms/preparedness-and-response/
65. Mathiesen M J, Christiansen M, Hansen K, Holm A, Asbrink E, Theisen M. Peptide-based OspC enzyme-linked immunosorbent assay for serodiagnosis of Lyme borreliosis. J Clin Microbiol. 1998.
66. Panelius J, Lahdenne P, Heikkilä T, Peltomaa M, Oksi J, Seppälä I. Recombinant OspC from Borrelia burgdorferi sensu stricto, B. afzelii and B. garinii in the serodiagnosis of Lyme borreliosis. J Med Microbiol [Internet]. 2002 Sep. 1 [cited 2022 Jan. 1]; 51 (9): 731-9. Available from: www.microbiologyresearch.org/content/journal/jmm/10.1099/0022-1317-51-9-731
67. Padula S J, Dias F, Sampieri A, Craven R B, Ryan R W. Use of recombinant OspC from Borrelia burgdorferi for serodiagnosis of early Lyme disease. J Clin Microbiol. 1994; 32 (7): 1733-8.
68. Coleman A S, Pal U. BBK07, a dominant in vivo antigen of Borrelia burgdorferi, is a potential marker for serodiagnosis of Lyme disease. Clin Vaccine Immunol [Internet]. 2009 November [cited 2018 Jun. 17]; 16 (11): 1569-75. Available from: http://www.ncbi.nlm.nih.gov/pubmed/19776192
69. Coleman A S, Rossmann E, Yang X, Song H, Lamichhane C M, Iyer R, et al. BBK07 immunodominant peptides as serodiagnostic markers of lyme disease. Clin Vaccine Immunol. 2011; 18 (3): 406-13.
70. Signorino G, Arnaboldi P M, Petzke M M, Dattwyler R J. Identification of OppA2 linear epitopes as serodiagnostic markers for lyme disease. Clin Vaccine Immunol. 2014; 21 (5): 704-11.
71. Heikkila T, Seppälä I, Saxen H, Panelius J, Yrjänäinen H, Lahdenne P. Species-specific serodiagnosis of lyme arthritis and neuroborreliosis due to Borrelia burgdorferi sensu stricto, B. afzelii, and B. garinii by using decorin binding protein A. J Clin Microbiol. 2002; 40 (2): 453-60.
72. Arnaboldi P M, Sambir M, Dattwyler R J. Decorin binding proteins A and B in the serodiagnosis of lyme disease in North America. Clin Vaccine Immunol. 2014; 21 (10): 1426-36.
73. Oldenburg D G, Jobe D A, Lovrich S D, LaFleur R L, White D W, Dant J C, et al. Detection of antibodies to decorin-binding protein A (DbpA) and DbpB after infection of dogs with Borrelia burgdorferi by tick challenge. J Vet Diagnostic Investig. 2020; 32 (3): 481-5.
74. Sillanpaa H, et al. Immune response to borrelial VlsE IR6 peptide variants. International Journal of Medical Microbiology. 2007; 297 (1): 45-52.
75. Krupta I, et al. Borrelia burgdorferi Sensu Lato Species in Europe Induce Diverse Immune Responses against C6 Peptides in Infected Mice. Clinical and Vaccine Immunology. 2009; 16 (11): 1546-1562.
76. Christova I, et al. C6 peptides from Borrelia burgdorferi sensu stricto, Borrelia afzelii and Borrelia garinh as antigens for serological diagnosis of lyme borreliosis. Biotechnology and Biotechnological Equipment. 2013; 27 (1): 3452.
77. Wei X, et al. Development of Two Avidity-Based Assays to Detect Recent HIV Type 1 Seroconversion Using a Multisubtype gp41 Recombinant Protein. AIDS Research and Human Retroviruses. 2010; 26 (1): 61-71.

What is claimed is:

1. A method of detecting the presence of an antibody in a sample, the method comprising:
(i) contacting the sample with a first antigen immobilized to a surface of a vessel and incubating the sample with the first antigen;
(ii) contacting the sample with a second antigen conjugated to a detectable label and incubating the sample with the second antigen; and
(iii) assessing the presence of a signal from the detectable label;
wherein:
the presence of a signal is indicative of association of the antibody with the first antigen and the second antigen;
incubating the first antigen and the second antigen with the sample is sufficient to bind the first antigen to a first antigen-binding site on the antibody and the second antigen to a second antigen-binding site on the antibody, if present; and
the first antigen and the second antigen are different.

2. A method of detecting the presence of an antibody in a sample, the method comprising:
(i) adding the sample to a vessel comprising a surface coated with a first binding molecule;
(ii) adding a first antigen conjugated to a second binding molecule to the vessel and incubating the sample with the first antigen;
(iii) adding a second antigen conjugated to a detectable label to the vessel and incubating the sample with the second antigen; and
(iv) assessing the presence of a signal from the detectable label;
wherein:
the presence of a signal is indicative of association of the antibody with the first antigen and the second antigen;
incubating the first antigen and the second antigen with the sample is sufficient to bind the first antigen to a first antigen-binding site on the antibody and the second antigen to a second antigen-binding site on the antibody, if present;
the first binding molecule binds to the second binding molecule; and
the first antigen and the second antigen are different.

3. A method of detecting the presence of an antibody in a sample, the method comprising:
(i) incubating the sample with a first antigen to form an antibody-first antigen complex, wherein the first antigen is bound to a first binding molecule;
(ii) immobilizing a second antigen on a solid substrate;
(iii) adding the sample comprising the antibody-first antigen complex to the solid substrate;
(iv) incubating the solid substrate comprising the sample comprising the antibody-first antigen complex for a sufficient time to further bind the antibody-first antigen complex to the second antigen to form a second antigen-antibody-first antigen complex;
(v) adding a second binding molecule conjugated to a detectable label to the solid substrate; and
(vi) assessing the presence of a signal from the detectable label;
wherein:
the presence of a signal is indicative of association of both (1) the antibody with the first antigen and the second antigen and (2) the first binding molecule with the second binding molecule;
incubating the sample with the first antigen is sufficient to bind the first antigen to a first antigen-binding site on the antibody, if present;
incubating the sample with the second antigen immobilized on the solid substrate is sufficient to bind the second antigen to a second antigen-binding site on the antibody, if present; and
the first antigen and the second antigen are different.

4. A method of detecting the presence of an antibody in a sample, the method comprising:
(i) immobilizing a first antigen on a solid substrate;
(ii) adding a second antigen to the solid substrate, wherein the second antigen is conjugated to a detectable label or is adapted to be conjugated to the detectable label;
(iii) adding the sample to the solid substrate;
(iv) incubating the solid substrate comprising the sample, the first antigen and the second antigen for sufficient time to bind the second antigen to a first antigen-binding site on the antibody in the sample, if present;
(v) incubating the solid substrate comprising the sample, the first antigen and the second antigen for sufficient time to bind the first antigen to a second antigen-binding site on the antibody in the sample, if present; and
(vi) assessing the presence of a signal from the detectable label;
wherein:
the presence of a signal is indicative of association the antibody with the first antigen and the second antigen; and
the first antigen and the second antigen are different.

5. A system comprising:
a microplate comprising a plurality of wells; and
a first antigen immobilized or adapted to be immobilized to at least one of the plurality of wells;
wherein:
at least one of the plurality of wells including the first antigen is adapted to receive:
(i) a sample to be analyzed for the presence of an antibody; and
(ii) a second antigen conjugated to a detectable label;
wherein:
the first antigen binds to a first antigen-binding site on the antibody and the second antigen binds to a second antigen-binding site on the antibody, if present; and
the first antigen and the second antigen are different.

6. A microplate, comprising:
a plurality of wells; and
a first antigen immobilized to a surface of at least one of the plurality of wells;
wherein:
the microplate is adapted to receive:
a sample to be analyzed for the presence of an antibody; and
a second antigen conjugated to a detectable label in at least one of the plurality of wells including the first antigen;
the first antigen binds to a first antigen-binding site on the antibody and the second antigen binds to a second antigen-binding site on the antibody, if present; and
the first antigen and the second antigen are different.

7. A microplate, comprising:
a plurality of wells;
a first antigen immobilized to a surface of at least one of the plurality of wells;
a second antigen disposed in at least one of the plurality of wells including the first antigen; and
an antibody bound to the first antigen and the second antigen;
wherein:
the second antigen is conjugated to a detectable label; and
the first antigen and the second antigen are different.

8. A kit, comprising:
a microplate comprising a plurality of wells and a first antigen immobilized to a surface of at least one of the plurality of wells; and
a reagent comprising a second antigen conjugated to a detectable label;
wherein:
the microplate is adapted to receive a sample to be analyzed for the presence of an antibody; and
the first antigen and the second antigen are different.

9. A kit, comprising:
a microplate comprising a plurality of wells, wherein the plurality of wells are coated with a first binding molecule;
a reagent comprising a first antigen conjugated to a second binding molecule; and
a reagent comprising a second antigen conjugated to a detectable label;
wherein:
the microplate is adapted to receive a sample to be analyzed for the presence of an antibody; and
the first antigen and the second antigen are different.

10. A kit, comprising:
a solid substrate;
a reagent comprising a first antigen conjugated to a first binding molecule;
a reagent comprising a second antigen; and
a reagent comprising a second binding molecule conjugated to a detectable label;
wherein:
the substrate is adapted to receive a sample to be analyzed for the presence of an antibody; and
the first antigen and the second antigen are different.

11. A composition, comprising:
an antibody derived from a sample;
a first antigen bound to a first antigen-binding site;
a second antigen bound to a second antigen-binding site;
wherein:
the second antigen is conjugated to a detectable label; and
the first antigen and the second antigen are different.

12. A one-step method of detecting the presence of an antibody in a sample, the method comprising:
(i) contacting the sample with a first antigen immobilized to a surface of a vessel and incubating the sample with the first antigen;
(ii) contacting the sample with a second antigen conjugated to a detectable label and incubating the sample with the second antigen; and
(iii) assessing the presence of a signal from the detectable label;
wherein:
the presence of a signal is indicative of association of the antibody with the first antigen and the second antigen;
incubating the first antigen and the second antigen with the sample is sufficient to bind the first antigen to a first antigen-binding site on the antibody and the second antigen to a second antigen-binding site on the antibody, if present;
the first antigen and the second antigen are different; and
the method does not require a confirmatory test.

13. The method, system, microplate, kit, or composition of any one of the preceding claims, wherein the first antigen is a recombinant protein.

14. The method, system, microplate, kit, or composition of claim 13, wherein the second antigen is a peptide derived from the recombinant protein.

15. The method, system, microplate, kit, or composition of any one of the preceding claims, wherein the second antigen is a recombinant protein.

16. The method, system, microplate, kit, or composition of claim 15, wherein the first antigen is a peptide derived from the recombinant protein.

17. The method, system, microplate, kit, or composition of any one of the preceding claims, wherein the first antigen is selected from a group consisting of: VlsE, C6, OspC, C10, BBK07, OppA2, and Decorin Binding Protein A.

18. The method, system, microplate, kit, or composition of any one of the preceding claims, wherein the second antigen is selected from a group consisting of: VlsE, C6, OspC, C10, BBK07, OppA2, and Decorin Binding Protein A.

19. The method, system, microplate, kit, or composition of any one of the preceding claims, wherein the first antigen is VlsE and the second antigen is C6.

20. The method, system, microplate, kit, or composition of any one of the preceding claims, wherein the first antigen is C6 and the second antigen is VlsE.

21. The method, system, microplate, kit, or composition of any one of the preceding claims, wherein the first antigen is OspC and the second antigen is C10.

22. The method, system, microplate, kit, or composition of any one of the preceding claims, wherein the first antigen is C10 and the second antigen is OspC.

23. The method of any one of the preceding claims, wherein the first binding molecule is an avidin protein and the second binding molecule is a biotin.

24. The method of claim 23, wherein the avidin protein is NeutrAvidin or streptavidin.

25. The method of claim 24, wherein the avidin protein is streptavidin.

26. The method, system, microplate, or kit of any one of the preceding claims, wherein the sample is a biological sample.

27. The method, system, microplate, kit, or composition of any one of the preceding claims, wherein the biological sample is selected from the group consisting of: saliva, oral fluid, tears, urine, interstitial fluid, synovial fluid, and cerebrospinal fluid.

28. The method, system, microplate, kit, or composition of any one of the preceding claims, wherein the biological sample is blood.

29. The method, system, microplate, kit, or composition of any one of the preceding claims, wherein the biological sample is blood, serum, plasma, or a blood fraction.

30. The method, system, microplate, kit, or composition of any one of the preceding claims, wherein the blood is collected from a subject suspected of having a Lyme disease infection.

31. The method, system, microplate, kit, or composition of any one of the preceding claims, wherein the subject is a human or a non-human animal.

32. The method, system, microplate, kit, or composition of any one of the preceding claims, wherein the subject is a human.

33. The method, system, microplate, kit, or composition of any one of the preceding claims, wherein the sample is collected from a subject suspected of having a disease caused by a pathogenic member of the bacterial genus Borrelia.

34. The method, system, microplate, kit, or composition of any one of the preceding claims, wherein the pathogenic member of the bacterial genus Borrelia is Borrelia lonestari, Borrelia microti, Borrelia turcica, Borrelia coriaceae, Borrelia miyamotoi, Borrelia texasensis, Borrelia andersonii, Borrelia bavariensis, Borrelia bissettii, Borrelia californiensis, Borrelia kurtenbachii, Borrelia spielmanii, Borrelia tanukii, Borrelia afzelii, Borrelia turdi, Borrelia valaisiana, Borrelia americana, Borrelia carolinensis, Borrelia burgdorferi, Borrelia garinii, Borrelia lusitaniae, Borrelia japonic, or Borrelia sinica.

35. The method, system, microplate, kit, or composition of any one of the preceding claims, wherein the pathogenic member of the bacterial genus Borrelia is Borrelia burgdorferi.

36. The method, system, microplate, kit, or composition of any one of the preceding claims, wherein the first antigen is a peptide or protein comprising an amino acid sequence having at least 90% sequence identity to the amino acid sequence set forth in any one of SEQ ID NOs: 1-83.

37. The method, system, microplate, kit, or composition of any one of the preceding claims, wherein the first antigen is a protein comprising an amino acid sequence having at least 90% sequence identity to the amino acid sequence set forth in any one of SEQ ID NO: 1-6.

38. The method, system, microplate, kit, or composition of any one of the preceding claims, wherein the second antigen is a peptide or protein comprising an amino acid sequence having at least 90% sequence identity to the amino acid sequence set forth in any one of SEQ ID NOs: 1-83.

39. The method, system, microplate, kit, or composition of any one of the preceding claims, wherein the second antigen is a peptide comprising an amino acid sequence having at least 90% sequence identity to the amino acid sequence set forth in any one of SEQ ID NOs: 7-83.

40. The method, system, microplate, kit, or composition of any one of the preceding claims, wherein the sequence identity is at least 91%, at least 92%, at least 93%, at least 94%, at least 95%, at least 96%, at least 97%, at least 98%, at least 99%, at least 99.5%, or at least 100%.

41. The method of any one of the preceding claims, further comprising a wash step in between step (ii) and step (iii).

42. The method of claim 41, wherein the wash step comprises adding a wash buffer to the vessel and incubating the wash buffer in the vessel for a sufficient time to remove unbound antibodies.

43. The method, system, microplate, kit, or composition of any one of the preceding claims, wherein the detectable label is a luminescent label, a colorimetric label, or a radiometric label.

44. The method, system, microplate, kit, or composition of any one of the preceding claims, wherein the luminescent label is a luminescent dye, a FRET label, or a fluorescent protein.

45. The method, system, microplate, kit, or composition of any one of the preceding claims, wherein the luminescent label is a lanthanide, a fluorescent label, or an organic dye.

46. The method, system, microplate, kit, or composition of any one of the preceding claims, wherein the colorimetric label is a horseradish peroxidase label or alkaline phosphatase.

47. The method of any one of the preceding claims, further comprising a step of detecting the detectable label between step (ii) and step (iii).

48. The method, system, microplate, kit, or composition of any one of the preceding claims, wherein the step of assessing the presence of a signal comprises detecting signal pulses from the microplate wells.

49. The system, microplate, kit, or composition of any one of the preceding claims, wherein the detectable label is adapted to be detected by detecting a signal pulse emitted by the detectable label.

50. The method, system, microplate, kit, or composition of any one of the preceding claims, wherein the step of detecting comprises adding a substrate to the microplate wells.

51. The system, microplate, kit, or composition of any one of the preceding claims, wherein the detectable label is adapted to be detected by a substrate.

52. The method, system, microplate, kit, or composition of any one of the preceding claims, wherein the substrate is selected from the group consisting of: ABTS, OPD, AmplexRed, DAB, AEC, TMB, Homovanillic Acid, or Luminol.

53. The method, system, microplate, kit, or composition of any one of the preceding claims, wherein the substrate is 3,3′,5,5′-tetramethylbenzidine (TMB).

54. The method, system, microplate, kit, or composition of any one of the preceding claims, wherein the substrate is Luminol.

55. The method of any one of the preceding claims, wherein the vessel is adapted to receive at least a portion of the sample.

56. The method of any one of the preceding claims, wherein the vessel is a well.

57. The method of any one of the preceding claims, wherein the vessel is a well of a microplate comprising one or more wells.

58. The method of any one of the preceding claims, wherein the presence of a signal is also indicative of the presence of the antibody in the sample.

59. The method of any one of the preceding claims, wherein the presence of a signal is suggestive of a Lyme disease infection in the subject.

60. The method of any one of the preceding claims, wherein the presence of a signal is indicative of Lyme disease-specific antibodies.

61. The method of any one of the preceding claims, wherein the presence of a signal is indicative of a previous or active Lyme disease infection in the subject.

62. The method of any one of the preceding claims, wherein the presence of a signal is indicative of an active Lyme disease infection in the subject.

63. The method of any one of the preceding claims, further comprising administering an antibiotic to the subject from whom the sample was obtained.

64. The method of claim 63, wherein the antibiotic is selected from the group consisting of: amoxicillin, cefuroxime, doxycycline, and ceftriaxone.

65. The method of claim 64, wherein the antibiotic is doxycycline.

66. The method of any one of the preceding claims, wherein steps (i) and (ii) are performed simultaneously.

67. The method of any one of the preceding claims, wherein steps (ii) and (iii) are performed simultaneously.

68. The method, system, microplate, kit, or composition of any one of the preceding claims, wherein the first antigen-binding site and the second antigen-binding site are identical.

69. The method, system, microplate, kit, or composition of any one of the preceding claims, wherein the first antigen-binding site and the second antigen-binding site share at least 90% sequence identity.

70. The method, system, microplate, kit, or composition of any one of the preceding claims, wherein the first antigen-binding site and the second antigen-binding site share 91%, 92%, 93%, 94%, 95%, 96%, 97%, 98%, 99%, 99.5%, or 100% sequence identity.

71. The method, system, microplate, kit, or composition of any one of the preceding claims, wherein the first antigen is capable of binding to the first antigen-binding site or the second antigen-binding site.

72. The method, system, microplate, kit, or composition of any one of the preceding claims, wherein the second antigen is capable of binding to the first antigen-binding site or the second antigen-binding site.

73. The method of any one of the preceding claims, wherein step (i) is performed before step (ii).

74. The method of any one of the preceding claims, wherein step (ii) is performed before step (i).

75. The method of any one of the preceding claims, wherein step (ii) is performed before step (iii).

76. The method of any one of the preceding claims, wherein step (iii) is performed before step (ii).

77. The method, system, microplate, kit, or composition of any one of the preceding claims, wherein the first antigen is a first peptide and the second antigen is a second peptide.

78. The method of any one of the preceding claims, wherein the method does not require a confirmatory test.

79. The method, system, microplate, kit, or composition of any one of the preceding claims, wherein the disease to be detected is an infectious disease.

80. The method, system, microplate, kit, or composition of any one of the preceding claims, wherein the disease or infectious agent to be detected is selected from the group consisting of: Lyme disease, hepatitis, Chagas disease, and a human immunodeficiency virus infection.

81. The method, system, microplate, kit, or composition of any one of the preceding claims, wherein the first antigen is bound to a complex.

82. The method, system, microplate, kit, or composition of any one of the preceding claims, wherein the second antigen is bound to a complex.

83. The method, system, microplate, kit, or composition of any one of the preceding claims, wherein the conjugate is selected from the group consisting of: horseradish peroxidase (HRP), biotin, an avidin protein, a maleimide-activated HRP, or a maleimide.

84. The method of any one of the preceding claims, wherein the solid substrate is a membrane.

85. The method of any one of the preceding claims, wherein the membrane is a nitrocellulose membrane.

86. The method of any one of the preceding claims, wherein the membrane is a Fusion 5 membrane.

87. The method, system, microplate, kit, or composition of any one of the preceding claims, wherein the first antigen is a peptide comprising an amino acid sequence having at least 90% sequence identity to the amino acid sequence set forth in any one of SEQ ID NOs: 7-83.

88. The method, system, microplate, kit, or composition of any one of the preceding claims, wherein the second antigen is a protein comprising an amino acid sequence having at least 90% sequence identity to the amino acid sequence set forth in any one of SEQ ID NOS: 1-6.

89. The method of any one of the preceding claims, wherein the steps of the method are performed sequentially.

90. The method of any one of the preceding claims, wherein the steps of the method are performed simultaneously.

91. The method of any one of the preceding claims, wherein the steps of the method are performed in any order.

92. The method, system, microplate, kit, or composition of any one of the preceding claims, wherein the detectable label is conjugated to the first antigen.

93. The method, system, microplate, kit, or composition of any one of the preceding claims, wherein the detectable label is conjugated to the second antigen.

94. The method, system, microplate, kit, or composition of any one of the preceding claims, wherein the detectable label is conjugated to the first binding molecule.

95. The method, system, microplate, kit, or composition of any one of the preceding claims, wherein the detectable label is conjugated to the second binding molecule.

96. The method of any one of the preceding claims, wherein the first antigen is immobilized directly on the solid substrate.

97. The method of any one of the preceding claims, wherein the first antigen is immobilized to the solid substrate via a binding molecule pair.

98. The method of any one of the preceding claims, wherein the detectable label is conjugated directly to the second antigen.

99. The method of any one of the preceding claims, wherein the detectable label is conjugated to the second antigen via a binding molecule pair.

100. The method of any one of the preceding claims, wherein the binding molecule pair comprises a first binding molecule and a second binding molecule.

101. The method of any one of the preceding claims, further comprising the step of conjugating the detectable label to the second antigen.
